SEQUENCE LISTING (250 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 16 aa, synthetic construct
AKDRLSITIRPRYYGL

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, synthetic construct
RNAMF

SEQ ID NO: 67 - protein, 16 aa, synthetic construct
GIGTGGATSYADSVKG

SEQ ID NO: 68 - protein, 7 aa, synthetic construct
GRYYFDY

SEQ ID NO: 69 - protein, 12 aa, synthetic construct
RASQSVSSSYLA

SEQ ID NO: 70 - protein, 7 aa, synthetic construct
GASSRAT

SEQ ID NO: 71 - protein, 10 aa, synthetic construct
QQYDHSAGWT

SEQ ID NO: 72 - protein, 7 aa, synthetic construct
GASSRAP

SEQ ID NO: 73 - protein, 10 aa, synthetic construct
QQYGSSPPWT

SEQ ID NO: 74 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 77 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 78 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 79 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 80 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 81 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 82 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 83 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 84 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 85 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 90 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 91 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 92 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 93 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 95 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 96 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 97 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 98 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 99 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 100 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 101 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 103 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 104 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 105 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 106 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 108 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 109 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 110 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 111 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 112 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 113 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 114 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 115 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 116 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 117 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 118 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 119 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 120 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 121 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 122 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 123 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 124 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 125 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 126 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 127 - protein, 5 aa, synthetic construct
SYYMH

SEQ ID NO: 128 - protein, 17 aa, synthetic construct
IINPRGGSTSYAQKFQG

SEQ ID NO: 129 - protein, 7 aa, synthetic construct
GKYWMYD

SEQ ID NO: 130 - protein, 13 aa, synthetic construct
SGGGSSIGNSYVS

SEQ ID NO: 131 - protein, 7 aa, synthetic construct
DNNKRPS

SEQ ID NO: 132 - protein, 12 aa, synthetic construct
GTWDTSPVWEWP

SEQ ID NO: 133 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 134 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 135 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 136 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 137 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 138 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 139 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 140 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 141 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 142 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 143 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 144 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 145 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 146 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 147 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 148 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 149 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 150 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 151 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 152 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 153 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 154 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 155 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 156 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 157 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 158 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 159 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 160 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 164 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 165 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 166 - protein, 5 aa, synthetic construct
RHAMA

SEQ ID NO: 167 - protein, 17 aa, synthetic construct
AITSSGRSIYYADSVKG

SEQ ID NO: 168 - protein, 7 aa, synthetic construct
VHRAFDY

SEQ ID NO: 169 - protein, 13 aa, synthetic construct
SGSPLFPDSGSFN

SEQ ID NO: 170 - protein, 7 aa, synthetic construct
ADSHRPS

SEQ ID NO: 171 - protein, 11 aa, synthetic construct
GTWDYSLSGYV

SEQ ID NO: 172 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 173 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 174 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 175 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 176 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 177 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 178 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 179 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 180 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 181 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 182 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 183 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 184 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 185 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 186 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 187 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 188 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 189 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 190 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 191 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 192 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 193 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 194 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 195 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 196 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 197 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 198 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 199 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 200 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 201 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 202 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 203 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 204 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 205 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 206 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 207 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 208 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 209 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 210 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 211 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 212 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 213 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 214 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 215 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 216 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 217 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 218 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 219 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 220 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 221 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 222 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 223 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 224 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 225 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 226 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 227 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 228 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 229 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 230 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 231 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 232 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 233 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 234 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 235 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 236 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 237 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 238 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 239 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 240 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 241 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 242 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 243 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 244 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 245 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 246 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 247 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 248 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 249 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 250 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

(19) United States
(12) Patent Application Publication
(10) Pub. No.: US 2026/0347485 A1
(43) Pub. Date: Nov. 5, 2026

(54) METHODS FOR TREATING CHRONIC INDUCIBLE COLD URTICARIA BY ADMINISTERING AN IL-4R ANTAGONIST

(71) Applicant: SANOFI BIOTECHNOLOGY, Gentilly (FR)

(72) Inventors: Elizabeth LAWS, Cambridge, MA (US); Leda MANNENT, Paris (FR); Renata MARTINCOVA, Paris (FR); Aleksandra STJEPANOVIC, Paris (FR)

(21) Appl. No.: 19/104,869

(22) PCT Filed: Aug. 29, 2023
(86) PCT No.: PCT/EP2023/073630

§ 371 Date: Feb. 19, 2025

Related U.S. Application Data
(60) Provisional application No. 63/401,756, filed on Aug. 29, 2022.

(30) Foreign Application Priority Data
Feb. 2, 2023 (EP) 23315021.8

(51) Int. Cl. C07K 16/28; A61K 39/00; A61P 17/04

(52) U.S. Cl. CPC C07K 16/2866; A61P 17/04; A61K 2039/545; A61K 2039/55

(57) ABSTRACT

Methods for treating or preventing chronic inducible urticaria, e.g., chronic inducible cold urticaria (ColdU), in a subject are provided. Methods comprising administering to a subject in need thereof a therapeutic composition comprising an interleukin-4 receptor (IL-4R) antagonist, such as an anti-IL-4R antibody or antigen-binding fragment thereof, are provided.

### RELATED APPLICATIONS

This application is a 35 U.S.C. § 371 filing of International Patent Application No. PCT/EP2023/073630, filed Aug. 29, 2023, which claims the benefit of U.S. Provisional Application No. 63/401,756, filed Aug. 29, 2022, and European Patent Application No. 23315021.8, filed Feb. 2, 2023, the contents of which are incorporated by reference herein.

### SEQUENCE LISTING

The instant application contains a Sequence Listing which has been submitted electronically in XML format and is hereby incorporated by reference in its entirety. Said XML file, created on Feb. 18, 2025, is named 760520_SA9-331US_ST26.xml and is 241,313 bytes in size.

### FIELD OF THE INVENTION

The disclosure relates to the treatment and/or prevention of chronic inducible urticaria in a subject in need thereof. The disclosure relates to the administration of an interleukin-4 receptor (IL-4R) antagonist to treat or prevent chronic inducible urticaria in a subject in need thereof.

### BACKGROUND

Chronic inducible urticaria is a condition characterized by the appearance of wheals, angioedema, or both, as a response to a specific and reproducible trigger. Common forms of chronic inducible urticaria are physical urticarias, which are related to the exposure to physical triggers, such as cold, heat, vibration or pressure. The physical urticarias (e.g., heat and cold urticarias, symptomatic dermographism, delayed-pressure urticaria, solar urticaria and vibratory angioedema) are distinguished from other inducible urticarias such as cholinergic urticaria, contact urticaria and aquagenic urticaria. Chronic inducible urticaria symptoms are typically confined to skin areas that are exposed to the specific trigger. Chronic inducible urticarias are diagnosed based on patient history and the results of provocation testing. The aims of provocation testing are to determine the relevant trigger(s) in individual subjects and to assess trigger thresholds.

Among the group of physical urticarias, chronic inducible cold urticaria (ColdU) is defined as a physical urticaria characterized by a cold trigger to induce the formation of wheals and/or the onset of angioedema. ColdU is defined by the appearance of wheals after contact cooling and rewarming of the skin. In ColdU, symptoms usually develop within minutes after provocation using cold air, cold liquids, or cold solid objects in contact with the skin.

As used herein, “primary acquired ColdU” refers to a non-familial form of ColdU that is considered idiopathic in nature. Historically, the majority (96% of subjects with acquired ColdU have been shown to have primary ColdU, and only rare occurrences of secondary ColdU have been observed. (See Neittaanmaki (1985) J. Am. Acad. Dermatol. 13 (4): 636-44 for a review.)

Chronic inducible urticaria has an estimated prevalence of about 0.5% in the general population, and has substantial impact on quality of life (QoL) in many patients, mainly due to the need for trigger avoidance. Within the group of chronic inducible urticaria, most chronic inducible urticaria subtypes are rare and therefore very difficult to study. The most common are symptomatic dermographism, cholinergic urticaria and ColdU, chronic inducible ColdU being the second most common form of physical urticaria, with an estimated annual incidence of 0.05%.

ColdU is a debilitating condition that severely affects quality of life. It is normally not feasible to avoid the offending trigger without significant changes to everyday life. The majority of patients with ColdU experience systemic reactions, including anaphylaxis in severe cases. There is no approved treatment available to date for ColdU, and EAACI/GA²LEN/EDF/WAO guidelines treatment recommendations are primarily based on the approved treatments for chronic spontaneous urticaria (CSU). Many ColdU patients are refractory to these CSU treatments.

Therefore, there remains a high unmet need for novel therapies to treat chronic inducible urticarias such as ColdU.

### SUMMARY

In one aspect, a method for treating a subject having chronic inducible cold urticaria (ColdU) is provided comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein cold urticaria activity (ColdUAS) score is improved in the subject.

In certain exemplary embodiments, the subject remains symptomatic despite the use of H1 antihistamine.

In certain exemplary embodiments, an H1 antihistamine is administered in combination with the antibody or an antigen-binding fragment thereof. In certain exemplary embodiments, the H1 antihistamine is selected from the group consisting of cetirizine, levocetirizine, ebastine, fexofenadine, loratadine, desloratadine, bilastine, and rupatadine.

In certain exemplary embodiments, the subject is an adult. In certain exemplary embodiments, the antibody or antigen-binding fragment thereof is administered to the subject as an initial dose followed by one or more secondary doses. In certain exemplary embodiments, the initial dose is about 600 mg and each secondary dose is about 300 mg. In certain exemplary embodiments, each secondary dose is administered every 2 weeks.

In certain exemplary embodiments, the subject is 12 years old to less than 18 years old.

In certain exemplary embodiments, the antibody or antigen-binding fragment thereof is administered to the subject as an initial dose followed by one or more secondary doses.

In certain exemplary embodiments, the subject has a body weight greater than or equal to 30 kg and less than 60 kg and the initial dose is about 400 mg and each secondary dose is about 200 mg. In certain exemplary embodiments, each secondary dose is administered every 2 weeks.

In certain exemplary embodiments, the subject has a body weight of at least 60 kg and the initial dose is about 600 mg and each secondary dose is about 300 mg. In certain exemplary embodiments, each secondary dose is administered every 2 weeks.

In certain exemplary embodiments, the subject is 6 years old to less than 12 years old.

In certain exemplary embodiments, the antibody or antigen-binding fragment thereof is administered to the subject as an initial dose followed by one or more secondary doses.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject is 2 years old to less than 12 years old.

In certain exemplary embodiments, the antibody or antigen-binding fragment thereof is administered to the subject as an initial dose followed by one or more secondary doses.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject has a body weight of less than 30 kg and at least 15 kg and the initial dose is about 300 mg and each secondary dose is about 300 mg. In certain exemplary embodiments, each secondary dose is administered every 4 weeks.

In certain exemplary embodiments, the subject is at least 2 years old and less than 6 years old.

In certain exemplary embodiments, the subject has a body weight of less than 15 kg and at least 5 kg and the initial dose is about 200 mg and each secondary dose is about 200 mg. In certain exemplary embodiments, each secondary dose is administered every 4 weeks.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) is provided comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject was previously ineffectively treated with H1 antihistamine therapy, and wherein cold urticaria activity (ColdUAS) score is improved in the subject.

In certain exemplary embodiments, an H1 antihistamine is administered in combination with the antibody or an antigen-binding fragment thereof. In certain exemplary embodiments, the H1 antihistamine is selected from the group consisting of cetirizine, levocetirizine, ebastine, fexofenadine, loratadine, desloratadine, bilastine, and rupatadine.

In certain exemplary embodiments, the subject is an adult. In certain exemplary embodiments, the antibody or antigen-binding fragment thereof is administered to the subject as an initial dose followed by one or more secondary doses. In certain exemplary embodiments, the antibody or antigen-binding fragment thereof is administered to the subject as an initial dose followed by one or more secondary doses. In certain exemplary embodiments, the initial dose is about 600 mg and each secondary dose is about 300 mg. In certain exemplary embodiments, each secondary dose is administered every 2 weeks.

In certain exemplary embodiments, the subject is 12 years old to less than 18 years old.

In certain exemplary embodiments, the antibody or antigen-binding fragment thereof is administered to the subject as an initial dose followed by one or more secondary doses.

In certain exemplary embodiments, the subject has a body weight greater than or equal to 30 kg and less than 60 kg and the initial dose is about 400 mg and each secondary dose is about 200 mg. In certain exemplary embodiments, each secondary dose is administered every 2 weeks.

In certain exemplary embodiments, the subject has a body weight of at least 60 kg and the initial dose is about 600 mg and each secondary dose is about 300 mg. In certain exemplary embodiments, each secondary dose is administered every 2 weeks.

In certain exemplary embodiments, the subject is 6 years old to less than 12 years old.

In certain exemplary embodiments, the antibody or antigen-binding fragment thereof is administered to the subject as an initial dose followed by one or more secondary doses.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject is 2 years old to less than 12 years old.

In certain exemplary embodiments, the antibody or antigen-binding fragment thereof is administered to the subject as an initial dose followed by one or more secondary doses.

In certain exemplary embodiments, the subject has a body weight of at least 30 kg and the initial dose is about 400 mg and each secondary dose is about 200 mg. In certain exemplary embodiments, each secondary dose is administered every 2 weeks.

In certain exemplary embodiments, the subject has a body weight of less than 30 kg and at least 15 kg and the initial dose is about 600 mg and each secondary dose is about 300 mg. In certain exemplary embodiments, each secondary dose is administered every 4 weeks.

In certain exemplary embodiments, the subject has a body weight of less than 30 kg and at least 15 kg and the initial dose is about 300 mg and each secondary dose is about 300 mg. In certain exemplary embodiments, each secondary dose is administered every 4 weeks.

In certain exemplary embodiments, the subject is at least 2 years old and less than 6 years old.

In certain exemplary embodiments, the subject has a body weight of less than 15 kg and at least 5 kg and the initial dose is about 200 mg and each secondary dose is about 200 mg. In certain exemplary embodiments, each secondary dose is administered every 4 weeks.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject is 12 years old to less than 18 years old, the antibody or antigen-binding fragment thereof is administered to the subject as an initial dose followed by one or more secondary doses, and the antibody or antigen-binding fragment thereof is administered to the subject in a weight-dependent dosage, and wherein cold urticaria activity (ColdUAS) score is improved in the subject.

In certain exemplary embodiments, the subject has a body weight greater than or equal to 30 kg and less than 60 kg and the initial dose is about 400 mg and each secondary dose is about 200 mg.

In certain exemplary embodiments, the subject has a body weight of at least 60 kg and the initial dose is about 600 mg and each secondary dose is about 300 mg.

In certain exemplary embodiments, each secondary dose is administered every 2 weeks.

In certain exemplary embodiments, an H1 antihistamine is administered in combination with the antibody or an antigen-binding fragment thereof.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject is the subject is 12 years old to less than 18 years old, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with antihistamine therapy, and wherein cold urticaria activity (ColdUAS) score is improved in the subject.

In certain exemplary embodiments, the subject has a body weight greater than or equal to 30 kg and less than 60 kg and the initial dose is about 400 mg and each secondary dose is about 200 mg.

In certain exemplary embodiments, the subject has a body weight of at least 60 kg and the initial dose is about 600 mg and each secondary dose is about 300 mg.

In certain exemplary embodiments, each secondary dose is administered every 2 weeks.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject is the subject is 6 years old to less than 12 years old, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with H1 antihistamine therapy, and wherein cold urticaria activity (ColdUAS) score is improved in the subject.

In certain exemplary embodiments, the subject has a body weight of between 15 kg and less than 30 kg, and the initial dose is about 600 mg and each secondary dose is about 300 mg.

In certain exemplary embodiments, each secondary dose is administered every 4 weeks.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject has a body weight of between 30 kg and less than 60 kg, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with H1 antihistamine therapy, and wherein cold urticaria activity (ColdUAS) score is improved in the subject.

In certain exemplary embodiments, the initial dose is about 400 mg and each secondary dose is about 200 mg.

In certain exemplary embodiments, each secondary dose is administered every 2 weeks.

In certain exemplary embodiments, the subject is at least 6 years old but less than 12 years old.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject has a body weight of between 5 kg and less than 15 kg, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with H1 antihistamine therapy, and wherein cold urticaria activity (ColdUAS) score is improved in the subject.

In certain exemplary embodiments, the initial dose is about 200 mg and each secondary dose is about 200 mg.

In certain exemplary embodiments, each secondary dose is administered every 4 weeks.

In certain exemplary embodiments, the subject is at least 2 years old but less than 6 years old.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject has a body weight of between 15 kg and less than 30 kg, the subject is at least 2 years old but less than 6 years old, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with H1 antihistamine therapy, and wherein cold urticaria activity (ColdUAS) score is improved in the subject.

In certain exemplary embodiments, the initial dose is about 300 mg and each secondary dose is about 300 mg.

In certain exemplary embodiments, each secondary dose is administered every 4 weeks.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating a subject having chronic inducible cold urticaria (ColdU) comprising administering to the subject an antibody or an antigen-binding fragment thereof that specifically binds interleukin-4 receptor (IL-4R) is provided. The antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, the subject is the subject is 2 years old to less than 12 years old, the antibody or antigen-binding fragment thereof is administered to the subject as an initial dose followed by one or more secondary doses, the antibody or antigen-binding fragment thereof is administered to the subject in a weight dependent dosage, and the subject was previously ineffectively treated with H1 antihistamine therapy, wherein cold urticaria activity (ColdUAS) score is improved in the subject.

In certain exemplary embodiments, the subject has a body weight of between 5 kg and less than 15 kg, and the initial dose is about 200 mg and each secondary dose is about 200 mg. In certain exemplary embodiments, each secondary dose is administered every 4 weeks. In certain exemplary embodiments, the subject is at least 2 years old but less than 6 years old.

In certain exemplary embodiments, the subject has a body weight of between 15 kg and less than 30 kg, and the initial dose is about 300 mg and each secondary dose is about 300 mg. In certain exemplary embodiments, each secondary dose is administered every 4 weeks. In certain exemplary embodiments, the subject is at least 2 years old but less than 6 years old.

In certain exemplary embodiments, the subject has a body weight of between 15 kg and less than 30 kg, and the initial dose is about 300 mg and each secondary dose is about 300 mg. In certain exemplary embodiments, each secondary dose is administered every 4 weeks.

In certain exemplary embodiments, the subject has a body weight of between 30 kg and less than 60 kg, and the initial dose is about 400 mg and each secondary dose is about 200 mg. In certain exemplary embodiments, each secondary dose is administered every 2 weeks.

In certain exemplary embodiments, the subject remains symptomatic despite the use of H1 antihistamine.

In certain exemplary embodiments, an H1 antihistamine is administered in combination with the antibody or an antigen-binding fragment thereof.

In certain exemplary embodiments, the H1 antihistamine is selected from the group consisting of cetirizine, levocetirizine, fexofenadine, loratadine, desloratadine, bilastine, and rupatadine.

In certain exemplary embodiments, the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

In certain exemplary embodiments, the improvement in the one or more ColdU-associated measures occurs with 12 weeks of treatment with the antibody or antigen-binding fragment thereof.

In certain exemplary embodiments, the improvement in the one or more ColdU-associated measures occurs with 24 weeks of treatment with the antibody or antigen-binding fragment thereof.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, the dosage of oral corticosteroids required is decreased.

In certain exemplary embodiments, the number of days wherein oral corticosteroid treatment is required is decreased.

In certain exemplary embodiments, the treatment with the antibody or antigen-binding fragment thereof results in a decrease in the need for treatment of the subject with antihistamine rescue medication.

In certain exemplary embodiments, the dosage of antihistamine rescue medication required is decreased.

In certain exemplary embodiments, the number of days wherein antihistamine rescue medication is required is decreased.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the antibody or antigen-binding fragment thereof is administered using an autoinjector, a needle and syringe, or a pen.

In certain exemplary embodiments, the antibody or antigen-binding fragment thereof is administered using a prefilled device.

In certain exemplary embodiments, the antibody or antigen-binding fragment thereof is administered subcutaneously.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

In another aspect, a method for treating chronic inducible cold urticaria (ColdU) in a subject is provided comprising selecting a subject having ColdU, and administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein cold urticaria activity (ColdUAS) score is improved in the subject.

In certain exemplary embodiments, prior to treatment with the antibody or antigen-binding fragment thereof, the subject has angioedema.

In certain exemplary embodiments, the treatment with the antibody or antigen-binding fragment thereof results in an increase in the number of itch-free days that the subject experiences.

In certain exemplary embodiments, the treatment with the antibody or antigen-binding fragment thereof results in an increase in the number of hive-free days that the subject experiences.

In certain exemplary embodiments, the treatment with the antibody or antigen-binding fragment thereof results in a decrease in the need for treatment of the subject with oral corticosteroids.

In certain exemplary embodiments, ColdUAS score is measured by sign and symptom free days on cold exposure days or is measured by sign and symptom severity score on cold exposure days.

In certain exemplary embodiments, ColdUAS score is decreased with 24 weeks of treatment with the antibody or antigen-binding fragment thereof.

In certain exemplary embodiments, total serum IgE is reduced in the subject.

In certain exemplary embodiments, the total serum IgE is reduced with 24 weeks of treatment with the antibody or antigen-binding fragment thereof.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 schematically depicts the study design for the EFC16720 study of Example 1. Adults and adolescents ≥60 kg are administered dupilumab 300 mg Q2W, administered as 1 SC injection of dupilumab 300 mg (2 mL). Adolescents <60 kg are administered dupilumab 200 mg Q2W, administered as 1 SC injection of dupilumab 200 mg (1.14 mL). Matched placebo is prepared in the same formulation without the addition of protein (i.e., the active substance). A loading dose equivalent to treatment group assigned is administered on day 1. Abbreviations: EOS: end of study; EOT: end of treatment; Q2W: every 2 weeks; N: number of participants; R: randomization; SC: subcutaneous.

FIG. 2 is a table showing the Schedule of Activities (SoA) for the EFC16720 study. Abbreviations: ACUSI=Acquired Cold Urticaria Severity Index; ADA=antidrug antibodies; AE=adverse event; AESI=adverse event of special interest; CDLQI=children's dermatology life quality index; ColdUAS=Cold Urticaria Activity Scale; ColdU-QOL=Cold Urticaria Quality of Life; DLQI=dermatology life quality index; DNA=deoxyribonucleic acid; ECG=electrocardiogram; eCRF=electronic Case Report Form; e-diary=electronic diary, EOS=End of study; EOT=End of treatment; EQ-5D 5L=5-level EuroQol 5-dimensional questionnaire; HBc Ab=hepatitis B core antibody; HBs Ab=hepatitis B surface antibody; HBs Ag=hepatitis B surface antigen; HBV=hepatitis B virus; HCRU=Healthcare resource utilization; HCV Ab=hepatitis C virus antibodies; HIV=Human Immunodeficiency Virus; IgE=immunoglobulin E; IMP=investigational medicinal product; IVRS=interactive voice response system; IWRS=interactive web response system; NRS, Numerical Rating Scale; OCS=oral corticosteroids; PGIC=patient global impression of change; PGIS=patient global impression of severity; PK=pharmacokinetic; q2w=every 2 weeks; RNA=ribonucleic acid; SAE=serious adverse event; SC=subcutaneous; TB=tuberculosis; UCT=urticaria control test; Ur=urine.

FIG. 3 schematically depicts the EFC16720 study parameters upon completion as set forth in Example 3.

FIG. 4 graphically depicts mean change from baseline in the proportion of cold urticaria sign and symptom free days over time up to week 24 on cold exposure days over 14 days observation period in an intent-to-treat (ITT) population.

FIG. 5 graphically depicts mean change from baseline in cold urticaria signs and symptoms severity over time up to week 24 on cold exposure days over 14 days observation period in an ITT population.

FIG. 6 graphically depicts median change (with Interquartile range) from baseline in total serum IgE (IU/mL) over time in a safety population.

FIG. 7 graphically depicts the placebo response in patients with baseline Likert 2. These patients had milder disease compared to other subgroups.

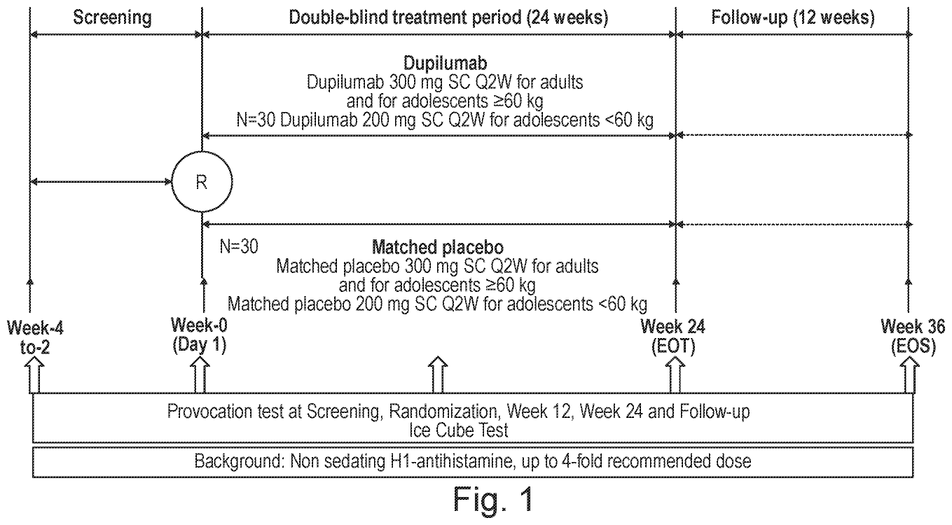

### DETAILED DESCRIPTION

Before the disclosure is described, it is to be understood that disclosure is not limited to particular methods and experimental conditions described, as such methods and conditions may vary. It is also to be understood that the terminology used herein is for the purpose of describing particular embodiments only, and is not intended to be limiting, because the scope of the disclosure will be limited only by the appended claims.

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the disclosures herein, the typical methods and materials are now described. All publications mentioned herein are incorporated herein by reference in their entirety.

The present disclosure provides methods and compositions for treating chronic inducible urticaria (e.g., chronic inducible cold urticaria (ColdU)) in a subject, e.g., in a human subject.

As used herein, “urticaria” refers to a skin condition characterized by the formation of wheal(s) (i.e., hive(s)) and/or the onset of angioedema that may last for a few minutes or many hours. As used herein “chronic urticaria” or “CU” refers to urticaria defined by recurrent episodes occurring at least twice a week for 6 weeks.

As used herein, “chronic spontaneous urticaria” or “CSU” refers to a subset of CU in which wheal(s) and/or angioedema are induced or provoked in a subject, over a period of at least six weeks, wherein the CSU has no specific cause or trigger.

As used herein, “chronic inducible urticaria” refers to a subset of CU in which wheal(s) and/or angioedema are induced or provoked in a subject, over a period of at least six weeks, by exposure to a specific trigger. There are nine subtypes of chronic inducible urticaria, depending on the type of trigger that provokes the chronic urticaria: symptomatic dermographism/urticaria factitia, cold urticaria, delayed pressure urticaria, solar urticaria, heat urticaria, vibratory angioedema, cholinergic urticaria, contact urticaria and aquagenic urticaria. Most chronic inducible urticaria subtypes are rare, but the most common are symptomatic dermographism/urticaria factitia, cholinergic urticaria, and chronic inducible cold urticaria.

In subjects with chronic inducible urticaria, wheal(s) and/or angioedema reproducibly develop in a subject in response to a trigger stimulus that is specific for the condition (e.g., upon cold exposure for cold contact urticaria). chronic inducible urticaria is typically diagnosed based on the subject history and the results of provocation testing (e.g., the formation of wheal(s) and/or the onset of angioedema). Subjects with severe chronic inducible urticaria may develop one or more systemic symptoms including, but not limited to, dizziness, vertigo, abdominal pain, vomiting, diarrhea, gastrointestinal ulcers, shortness of breath, wheezing, rapid and irregular heartbeat, anaphylactic shock, hypotension, shock, collapse and death.

As used herein, each of the following, “chronic inducible cold urticaria” (“ColdU” or “CICU”), and “cold inducible urticaria” (“CIndU”), are used interchangeably and refer to the development of itchy wheals and/or angioedema in a subject subsequent to exposure of the skin to cold. For a review of ColdU, see Magerl et al. (2016) Allergy 71:780-802 (doi: 10.1111/all.12884), incorporated herein by reference in its entirety for all purposes.

As used herein, a “wheal” refers to a raised, itchy (i.e., pruritic) area of the skin. Wheal(s) may be used interchangeably with “hive(s).” Wheal intensity may be characterized using a variety of assessment tools known in the art, including those discussed below.

As used herein, “angioedema” refers to an area of swelling of the lower layer of skin and tissue just under the skin or mucous membranes. Swelling may occur, e.g., in the face, tongue, larynx, abdomen, arms and/or legs. Onset is typically over minutes to hours and typically resolves in hours to a few days.

### Methods for Improving ColdU-Associated Measures

Methods for improving one or more ColdU-associated measures in a subject in need thereof, wherein the methods comprise administering a pharmaceutical composition comprising an IL-4R antagonist to the subject, are also provided.

Examples of ColdU-associated clinical-reported outcome (CRO) and patient-reported outcome (PRO) measures include, but are not limited to: (1) ice cube provocation test score; (2) wheal intensity Likert scale score; (3) peak pruritus numerical rating scale (NRS) score; (4) peak pain NRS score; (5) peak burning sensation NRS score; (6) urticaria control test (UCT) 4-item version score; (7) cold urticaria activity (ColdUAS) score; (8) children's dermatology quality life quality index (CDLQI) score; (9) infants' dermatitis quality of life index (IDQOL) score; (10) patient global impression of change (PGIC) score; (11) patient global impression of severity (PGIS) score; (12) acquired cold urticaria severity index (AColdUSI) score; (13) 5-level EuroQol 5-dimensional questionnaire (EQ-5D-5L) score; and (14) health care resource utilization/productivity score.

An “improvement in a ColdU-associated measure” means improvement from baseline of one or more of ice cube provocation test score, wheal intensity Likert scale score, NRS, peak pain NRS score, peak burning sensation NRS score, UCT 4-item version score, ColdUAS score, CDLQI score, IDQOL score, PGIC score, PGIS score, AColdUSI score, EQ-5D-5L score, and health care resource utilization/productivity score. As used herein, the term “baseline,” with regard to a ColdU-associated measure, means the numerical value of the measure for a patient prior to or at the time of administration of a pharmaceutical composition comprising an IL-4R antagonist.

To determine whether a ColdU-associated parameter has “improved,” the parameter is quantified at baseline and at a time point after administration of the pharmaceutical composition described herein. For example, a ColdU-associated parameter may be measured at day 1, day 2, day 3, day 4, day 5, day 6, day 7, day 8, day 9, day 10, day 11, day 12, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the ColdU-associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as a ColdU-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

### Efficacy Assessments

Ice Cube Provocation Test: According to certain embodiments, administration of an IL-4R antagonist to a patient results in an improvement from baseline in an ice cube provocation test. ColdU is defined by the appearance of signs and symptoms (hives/wheals, itch, pain, and burning sensation) after cold exposure and rewarming of the skin. Cold provocation tests are used in clinical studies in patients with chronic urticaria to evaluate efficacy of treatments either by evaluating the proportion of patients who after treatment did not develop signs/symptoms with the cold provocation test or evaluating the response to an experimental cold simulation time test, i.e., minimum time threshold of cold stimulation required to induce a coalescent wheal and other ColdU signs and symptoms.

The ice cube provocation test is the most frequently used provocation method for ColdU in routine clinical practice. The consensus recommendations for ColdU (EAACI/GA²LEN/EDF/UNEV) defines the following for provocation methods for ColdU:
Provocation tests should be performed by applying a cold stimulus (ice cube, cool packs) to forearm skin.
Cold provocation testing should be performed for 1 to 5 minutes on volar forearm.
Ice cube should be melting within a thin plastic bag to avoid cold damage of the skin and to prevent direct water contact to avoid the confusion with aquagenic urticaria if the test is positive.
Reading time: 10 minutes of rewarming after ice cube is removed.

A negative test is defined as the absence of a confluent hive/wheal at the entire skin site of exposure after ice cube provocation test. In some embodiments, administration of an IL-4R antagonist to a subject in need thereof results in the subject having a negative result in an ice cube provocation test.

Wheal Intensity Likert Scale: According to certain embodiments, administration of an IL-4R antagonist to a patient results in an improvement from baseline in wheal intensity Likert scale score. In certain embodiments, wheal intensity is assessed using a clinician-reported Likert scale ranging from 0 to 5, to categorize the intensity of subjects' cutaneous reaction rated as follows: 0=no wheals; 1=numerous small, non-coalescent wheals; 2=a large, regular, slightly edematous, coalescent wheal; 3=a large and moderately edematous wheal; 4=a large, regular, and significantly edematous wheal without pseudopodia; and 5=a large, very edematous wheal with pseudopodia. Score 0-1 corresponds to negative ice cube test. Score 2-5 corresponds to positive ice cube test The scale should be completed at the study visit, approximately 10 minutes after a temperature provocation test (e.g., a cold provocation test) is performed.

A negative provocation test is defined by the absence of a confluent hive/wheal at the entire skin site of exposure after a temperature provocation test (e.g., a cold provocation test) as described herein (i.e., no ColdU diagnosis). A positive provocation test is defined as the presence of at least a confluent hive/wheal at the entire skin site of exposure after a temperature (e.g., cold) provocation test (i.e., a positive ColdU diagnosis). Based on the score of the wheal intensity Likert scale, a score of 0-1 indicates a negative test result (i.e., no ColdU diagnosis), and a score of 2-5 indicates a positive test result (i.e., a positive ColdU diagnosis).

Therapeutic methods are provided that result in a decrease in wheal intensity Likert scale score from baseline. For example, administration of an IL-4R antagonist to a subject in need thereof results in a decrease in wheal intensity Likert scale score from baseline of about 1, 2, 3, 4, or 5.

Peak Pruritis Numerical Rating Scale (NRS): According to certain embodiments, administration of an IL-4R antagonist to a patient results in a decrease from baseline of peak pruritis NRS score. The peak pruritus NRS is a PRO comprised of a single item rated on a scale from 0 (“no itch”) to 10 (“worst itch imaginable”). In certain embodiments, subjects are asked to rate the intensity of their worst local site pruritus (itch) 10 minutes after a temperature provocation test (e.g., a cold provocation test) is performed. The 24-hour version of the scale has been developed, tested and validated with patients with atopic dermatitis. A threshold value of 4 has been determined as a meaningful within-person change in score in adults and adolescents patients with atopic dermatitis.

Therapeutic methods are provided that result in a decrease in peak pruritis NRS from baseline. For example, administration of an IL-4R antagonist to a subject in need thereof causes a decrease in peak pruritis NRS score from baseline of about 1, 2, 3, 4, 5, 6, 7, 8, 9, or 10.

Peak Pain Numerical Rating Scale (NRS): According to certain embodiments, administration of an IL-4R antagonist to a patient results in a decrease from baseline of peak pain NRS score. The peak pain NRS is a PRO comprised of a single item rated on a scale from 0 (“no pain”) to 10 (“worst imaginable pain”). In certain embodiments, subjects are asked to rate the intensity of their worst local site pain 10 minutes after a temperature provocation test (e.g., a cold provocation test) is performed.

Therapeutic methods are provided that result in a decrease in peak pain NRS from baseline. For example, administration of an IL-4R antagonist to a subject in need thereof causes a decrease in peak pain NRS score from baseline of about 1, 2, 3, 4, 5, 6, 7, 8, 9, or 10.

Peak Burning Sensation Numerical Rating Scale (NRS): According to certain embodiments, administration of an IL-4R antagonist to a patient results in a decrease from baseline of peak burning sensation NRS score. The peak burning sensation NRS is a PRO comprised of a single item rated on a scale from 0 (“no burning sensation”) to 10 (“worst imaginable burning sensation”). In certain embodiments, subjects will be asked to rate the intensity of the worst local site burning sensation of their skin 10 minutes after a temperature provocation test (e.g., a cold provocation test) is performed.

Therapeutic methods are provided that result in a decrease in peak pruritis NRS from baseline. For example, administration of an IL-4R antagonist to a subject in need thereof causes a decrease in peak pruritis NRS score from baseline of about 1, 2, 3, 4, 5, 6, 7, 8, 9, or 10.

Urticaria control test: According to certain embodiments, administration of an IL-4R antagonist to a patient results in an increase from baseline of the urticaria control test score. The urticaria control test (UCT) is a validated PRO measure for assessing urticaria control (Weller K, et al. Development, validation, and initial results of the Angioedema Activity Score. Allergy. 2013; 68 (9): 1185-92.) based on 4 items (severity of pruritus and wheals urticaria symptoms; frequency of treatment being not sufficient; QoL impairment; overall urticarial control). Each item is rated on a 5-point Likert-type scale (scored with 0 to 4 points). Low scores indicate high disease activity and low disease control. The UCT total score is calculated by adding all 4 individual item scores. Accordingly, the minimum and maximum UCT scores are 0 and 16, with a score of 16 points indicating complete disease control (See Weller K, et al. Development, validation, and initial results of the Angioedema Activity Score. Allergy. 2013; 68 (9): 1185-92.) A UCT score of 12 or greater indicates well controlled disease. The minimal important difference (MID) refers to the smallest change in a treatment outcome that an individual patient would identify as important and which would indicate a change in the patient's management, the MID value for UCT is 3.

Therapeutic methods are provided that result in an increase in UCT score from baseline. For example, administration of an IL-4R antagonist to a subject in need thereof causes an increase in UCT score from baseline of about 1, 2, 3, 4, 5, 6, 7, 8, 9, 10, 11, 12, 13, 14, 15, or 16 points.

Cold Urticaria Activity (ColdUAS) Score: According to certain embodiments, administration of an IL-4R antagonist to a patient results in a decrease from baseline of the ColdUAS score. The ColdUAS is a disease-specific PRO questionnaire designed to determine cold urticaria disease activity. ColdUAS is intended for patients with cold urticaria aged 12 years old and above; it has been developed and comprehensively tested with adults and adolescent patients with cold urticaria. Disease activity assessment is based on the daily documentation of cold-induced skin reactions (wheals and swelling), skin sensations (itching, burning, pain or feeling hot), avoidance behavior and trigger exposure, and overall symptoms severity. Skin reaction, skin sensations, exposition to cold temperatures that usually cause ColdU symptoms and overall symptom severity are rated on a 4-point scale from 0 (“No”) to 4 (“Yes, severe”); avoidance of cold temperatures that usually lead to ColdU symptoms are answered using responses “No,” “Yes partially avoided,” “Yes completely avoided.” Daily ColdUAS can be summed over a 7-day period to create a ColdUAS7 score.

After each week of completion, participants will be asked about the overall disease activity of their ColdU in the past week using a 4-point Likert scale from “No disease activity” to “High disease activity.” After each 2 weeks of completion, participants will be asked about the overall disease activity of their ColdU in the past 2 weeks using a 4-point Likert scale from “No disease activity” to “High disease activity,” and they should rate disease activity in the second week compared with first week from “markedly higher than in the first week” to “markedly lower than in the first week.”

Therapeutic methods are provided that result in a decrease in ColdUAS or ColdUAS7 score from baseline. For example, administration of an IL-4R antagonist to a subject in need thereof causes a decrease in ColdUAS score from baseline of about 1, 2, 3 or 4.

Children's Dermatology Quality Life Quality Index (CDLQI): According to certain embodiments, administration of an IL-4R antagonist to a patient results in a decrease from baseline of the CDLQI score. The Children's Dermatology Quality Life Quality Index (CDLQI) is a validated questionnaire designed to measure the impact of skin disease on children's HRQOL (See Lewis-Jones M S, Finlay A Y. The children's dermatology life quality index (CDLQI): initial validation and practical use. Br J Dermatol. 1995; 132 (6): 942-9). Patients provide responses to 10 questions (symptoms feelings associated with disease, the impact of the disease on leisure, school or holidays, personal relationships, sleep, and side effects of treatment for the skin disease). The instrument has a recall period of 7 days. Nine of the 10 questions are scored on a 4-point Likert scale ranging from 0=Not at all/question unanswered to 3=Very much. Question 7 has an additional possible response (prevented school), which is assigned a score of 3. The CDLQI total score is the sum of the score of each question with a maximum of 30 and a minimum of 0. The higher the score, the greater the impact is on the child's HRQOL. Patients complete the DLQI (≥16 years old) or CDLQI (≥12-<16).

Therapeutic methods are provided that result in a decrease in CDLQI score from baseline. For example, administration of an IL-4R antagonist to a subject in need thereof causes a decrease in CDLQI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Infants' Dermatitis Quality of Life Index (IDQOL): The IDQOL is a validated questionnaire designed for use in children aged <4 years. The questionnaire is completed by the child's caregiver/guardian. The instrument has a recall period of 1 week (7 days). There are 11 questions in total, 10 focusing on the topic of Life Quality Index scored on a 4-point Likert Scale plus an additional question scored on a 5-point Likert Scale focusing on Dermatitis Severity. For the Life Quality Index, questions 1, 5-10, the scoring is: all the time=3 to none=0. For question 2, the scoring is: always crying, etc.=3, very fretful=2, slightly fretful=1, happy=0. For question 3, the scoring is: more than 2 hours=3, 1 to 2 hours=2, 15 minutes to 1 hour=1, 0 to15 minutes=0. For question 4, the scoring is: 5 hours or more=3, 3 to 4 hours=2, 1 to 2 hours=1, Less than 1 hour=0. For the dermatitis severity, the 5-point Likert Scale scoring is: extremely severe=4 to none=0. The IDQOL total score is the sum of the score of each question with a maximum of 30 and a minimum of 0. The higher the score, the greater the impact is on the child's HRQoL.

Patient Global Impression of Change (PGIC): According to certain embodiments, administration of an IL-4R antagonist to a patient results in a decrease from baseline of PGIC score. The Patient Global Impression of Change (PGIC) is a 1-item questionnaire that asks the participant to provide the overall self-assessment of change in their CSU on a 7-point scale, compared to just before participant started taking the study treatment. Response choices are: 0=“very much better,” 1=“moderately better,” 2=“a little better,” 3=“no change,” 4=“a little worse,” 5=“moderately worse,” 6=“very much worse.” (See Guy W et al. ECDEU Assessment Manual for Psychopharmacology. Rockville, MD: US Department of Health, Education, and Welfare Public Health Service Alcohol, Drug Abuse, and Mental Health Administration, 1976.)

Therapeutic methods are provided that result in a decrease in PGIC score from baseline. For example, administration of an IL-4R antagonist to a subject in need thereof causes a decrease in PGIC score from baseline of about 1, 2, 3, 4, 5, or 6.

Patient Global Impression of Severity (PGIS): According to certain embodiments, administration of an IL-4R antagonist to a patient results in a decrease from baseline of PGIS score. The Patient Global Impression of Severity (PGIS) is a 1-item questionnaire that asks participants to provide the overall self-assessment of their participant's disease severity on a 4-point scale for the past week. Response choices are: 1=“none,” 2=“mild,” 3=“moderate,” 4=“severe.” (See Guy W et al. ECDEU Assessment Manual for Psychopharmacology. Rockville, MD: US Department of Health, Education, and Welfare Public Health Service Alcohol, Drug Abuse, and Mental Health Administration, 1976.)

Therapeutic methods are provided that result in a decrease in PGIS score from baseline. For example, administration of an IL-4R antagonist to a subject in need thereof causes a decrease in PGIS score from baseline of about 1, 2, or 3.

Acquired Cold Urticaria Severity Index (AColdUSI) Score: According to certain embodiments, administration of an IL-4R antagonist to a patient results in a decrease from baseline of AColdUSI score. The AColdUSI is a measure designed to evaluate the severity of the acquired cold urticarial (ACU) signs/symptoms. It is composed of 4 questions regarding the severity of ACU: 1) worst problems ever caused by cold urticaria; 2) season during which problems with outdoor activities occur because it was too cold; 3) maximum treatment needed; 4) frequency of complains. Questions 1, 3, and 4 are attributed 1 to 4 points, and question 3 is attributed 1 to 3 points, thus resulting in a score ranging from 4 to 15. Scores of 4 to 7, 8 to 11, and 12 to 15 points indicate low, middle, and high ACU severity, respectively. A fifth question assesses the overall severity of the disease between mild, moderate, and severe.

Therapeutic methods are provided that result in a decrease in AColdUSI score from baseline. For example, administration of an IL-4R antagonist to a subject in need thereof causes a decrease in AColdUSI score from baseline of about 1, 2, 3, 4, 5, 6, 7, 8, 9, 10, 11, 12, 13, 14, or 15.

Euroqol-5 dimensions (EQ-5D) and EQ-5D Youth version (EQ-5D Y): According to certain embodiments, administration of an IL-4R antagonist to a patient results in an increase from baseline of EQ-5D or EQ-5D Y score. The Euroqol-5 dimensions (EQ-5D) is a standardized PRO measure of health status developed by the EuroQol Group in order to provide a simple, generic measure of health for clinical and economic appraisal. The adult version of the questionnaire is adapted to patients aged 16 and older. The EQ-5D consists of 2 parts: the descriptive system and the EQ visual analogue scale (EQ VAS). The EQ-5D 5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “inability to do the activity.” (See Herdman M, et al. Development and preliminary testing of the new five-level version of EQ-5D (EQ-5D-5L). Qual. Life Res. 2011; 20 (10): 1727-36.) The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state. The EQ VAS records the respondent's self-rated health on a vertical, VAS where the endpoints are labeled “best imaginable health state (100)” and “worst imaginable health state (0).” This information can be used as a quantitative measure of health outcome as judged by the individual respondents. The EQ-5D Youth version (EQ-5D Y) is administered to children ≥6 to <12 years old and adolescents 12 to 15 years old. (Wille N, et al. Qual. Life Res. 2010; 19 (6): 875-86.) The EQ-5D-Y is based on the EQ-5D-3L and essentially consists of 2 pages: the EQ-5D descriptive system and the EQ VAS. The EQ-5D-Y descriptive system comprises the following 5 dimensions: mobility, looking after myself, doing usual activities, having pain or discomfort and feeling worried, sad or unhappy. Each dimension has 3 levels: no problems, some problems and a lot of problems. The EQ VAS records the younger patient's self-rated health on a vertical VAS where the endpoints are labelled “The best health you can imagine” and “The worst health you can imagine.” Patients complete the EQ-5D Y or EQ-5D questionnaire.

Therapeutic methods are provided that result in an increase in EQ VAS score from baseline. For example, administration of an IL-4R antagonist to a subject in need thereof causes an increase in EQ VAS score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

Health Care Resource Utilization/Productivity Score: According to certain embodiments, administration of an IL-4R antagonist to a patient results in a decrease of missed school days or missed work days experienced by a subject. A questionnaire on health care resource utilization and productivity is used to record missed days of school (3 to 18 years old)/workdays (18 and above). For example, administration of an IL-4R antagonist to a subject in need thereof causes a decrease from baseline missed days of school or work experienced by a subject by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Itch-Free Days: According to certain embodiments, administration of an IL-4R antagonist to a patient results in an increase from baseline in itch-free days experienced by a subject. For example, administration of an IL-4R antagonist to a subject in need thereof causes an increase from baseline in itch-free days experienced by a subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Hive-Free Days: According to certain embodiments, administration of an IL-4R antagonist to a patient results in an increase from baseline in hive-free days experienced by a subject. For example, administration of an IL-4R antagonist to a subject in need thereof causes an increase from baseline in hive-free days experienced by a subject of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days per month.

Itch Severity Score: According to certain embodiments, administration of an IL-4R antagonist to a patient results in a decrease from baseline of itch severity score (ISS). ISS7 is defined as the sum of the daily ISS scores (ranging from 0=none to 3-intense) recorded at the same time of the day for a 7-day period. The ISS7 ranges from 0-21, with higher scores indicating worse disease. The minimal important difference (MID) for ISS7 is 4.5-5.

Therapeutic methods are provided that result in a decrease in ISS7 score from baseline. For example, administration of an IL-4R antagonist to a subject in need thereof causes a decrease in ISS7 score from baseline of about 1, 2, 3, 4, 5, 6, 7, 8, 9, 10, 11, 12, 13, 14, 15, 16, 17, 18, 19, 20, or 21 points.

Hive Severity Score: According to certain embodiments, administration of an IL-4R antagonist to a patient results in a decrease from baseline of hive severity score (HSS). HSS7 is defined as the sum of the daily HSS scores (ranging from 0=none to 3=more than 50 hives) recorded at the same time of the day for a 7-day period. The HSS7 ranges from 0-21, with higher scores indicating worse disease. The minimal important difference (MID) for HSS7 is 5-5.5.

Therapeutic methods are provided that result in a decrease in HSS7 score from baseline. For example, administration of an IL-4R antagonist to a subject in need thereof causes a decrease in HSS7 score from baseline of about 1, 2, 3, 4, 5, 6, 7, 8, 9, 10, 11, 12, 13, 14, 15, 16, 17, 18, 19, 20, or 21 points.

Urticaria activity score: According to certain embodiments, administration of an IL-4R antagonist to a patient results in a decrease from baseline of urticaria activity score (UAS). UAS The Urticaria Activity Score (UAS) is a validated patient-recorded outcome (PRO) measure. The daily UAS is the sum of the daily Hive Severity Score (HSS, ranging from 0=None to 3=more than 50 hives) and the daily Itch Severity Score (ISS, ranging from 0=None to 3=intense), the 2 key urticaria signs and symptoms which are wheals and itch. The daily UAS scores range from 0 to 6 point/day. Daily UAS scores are summed over 7-day period to create the UAS7, ranging from 0 to 42, and is composed of the HSS7 and ISS7 components. The UAS7 is an established and widely accepted PRO tool to prospectively measure CSU activity. (See Mlynek A, et al. “How to assess disease activity in patients with chronic urticaria” Allergy. 2008; 63 (6): 777-80.) It has been used in most clinical trials in CSU in the recent years as a main outcome parameter and medical practice. (See Maurer M, et al. Omalizumab for the treatment of chronic idiopathic or spontaneous urticaria. N Engl J Med. 2013; 368 (10): 924-35; Casale T B, et al. Similar efficacy with omalizumab in chronic idiopathic/spontaneous urticaria despite different background therapy. J Allergy Clin Immunol Pract. 2015; 3 (5): 743-50). A minimal important difference (MID) value ranging from 9.5 to 10.5 has been defined to help interpretation of the change in score in CSU participants. (See Hollis K, et al. Comparison of urticaria activity score over 7 days (UAS7) values obtained from once-daily and twice-daily versions: Results from the ASSURE-CSU study. Am J Clin Dermatol. 2018; 19 (2): 267-74; Hawro T, et al. The urticaria activity score-validity, reliability, and responsiveness. J Allergy Clin Immunol Pract. 2018; 6 (4): 1185-90; Mathias S D, et al. Evaluating the minimally important difference of the urticaria activity score and other measures of disease activity in patients with chronic idiopathic urticaria. Ann Allergy Asthma Immunol. 2012; 108 (1): 20-4.) The UAS7 ranges from 0-42, with higher scores indicating greater disease activity. Scores of 1-6 indicate well controlled urticaria. Scores of 7-15 indicate mild urticaria. Scores of 16-27 indicate moderate urticaria activity. Scores of 28-42 indicate severe urticaria activity. A UAS7 score of 6 or less is considered to indicate well controlled urticaria. A complete responder (no itch and no hives) has a UAS7 of 0.

Therapeutic methods are provided that result in a decrease in UAS or UAS7 score from baseline. For example, administration of an IL-4R antagonist to a subject in need thereof causes a decrease in UAS7 score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 points.

Angioedema Activity Score: According to certain embodiments, administration of an IL-4R antagonist to a patient results in a decrease from baseline of angioedema activity score (AAS). The angioedema activity score (AAS) is a validated PRO measure that assesses angioedema activity (See Weller K, et al. Development, validation, and initial results of the Angioedema Activity Score. Allergy. 2013; 68 (9): 1185-92.) The AAS includes patients documenting the presence or absence of angioedema during the past 24 hours. If angioedema is present, patients answer 5 additional questions about the time of the day the swelling episode occurred, and the severity and impact on daily functioning and appearance this swelling episode has had. Each AAS item is scored between 0 and 3 points, that is, the minimum and maximum daily AASs are 0 and 15 points. The daily AASs are summed up to 7-day scores (AAS7), with 7-day scores ranging from 0 to 105 (Id.). A MID of the AAS7 of around 8 points has been established (Id).

Therapeutic methods are provided that result in a decrease in AAS or AAS7 score from baseline. For example, administration of an IL-4R antagonist to a subject in need thereof causes a decrease in AAS or AAS7 score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, or 105 points.

Dermatology life quality index (DLQI): According to certain embodiments, administration of an IL-4R antagonist to a patient results in a decrease from baseline of the DLQI score. The Dermatology life quality index (DLQI) is a PRO developed to measure dermatology-specific HRQOL in adult participants (See Finlay A Y, Khan G K. Dermatology life quality index (DLQI): a simple practical measure for routine clinical use. Clin Exp Dermatol. 1994; 19:210-6.) The instrument comprises 10 items assessing the impact of skin disease on participants' health-related quality of life (HRQOL) over the previous week. The items cover symptoms, leisure activities, work/school or holiday time, personal relationships including intimate, the side effects of treatment, and emotional reactions to having a skin disease. It is a validated questionnaire used in clinical practice and clinical trials (See Chernyshov P V. The evolution of quality of life assessment and use in dermatology. Dermatology. 2019; 235 (3): 167-74.) Response scale is a 4-point Likert scale (0=“not at all” and 3=“very much”) for 9 items. The remaining 1 item about work/studying asks whether work/study has been prevented and then (if “No”) to what degree the skin condition has been a problem at work/study; the item is rated on a 3-point Likert scale (‘Not at all’ to ‘A lot’). Overall scoring ranges from 0 to 30, with a high score indicative of a poor HRQOL. Using an integrated analysis of distribution and anchor-based approaches using the change in DLQI total score and participant-assessed itch severity scores, the MID for the DLQI in participants with chronic idiopathic urticaria was reported to be in the range of 2.24 to 3.10 points (Shikiar R, et al. Minimal important difference (MID) of the dermatology life quality index (DLQI): results from patients with chronic idiopathic urticaria. Health Qual. Life Outcomes. 2005; 3:36).

Therapeutic methods are provided that result in a decrease in DLQI score from baseline. For example, administration of an IL-4R antagonist to a subject in need thereof causes a decrease in DLQI score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

Chronic urticaria quality of life questionnaire (CU-Q20L): According to certain embodiments, administration of an IL-4R antagonist to a patient results in a decrease from baseline of the CU-Q2oL score. The CU-Q2oL is a disease-specific instrument used to assess the QoL in adult participants with CSU. (See Baiardini I, et al. A new tool to evaluate the impact of chronic urticaria on quality of life: chronic urticaria quality of life questionnaire (CU-QOL). Allergy. 2005; 60 (8): 1073-8.) The CU-Q2oL is a 23-item, self-administered questionnaire that includes 6 QoL dimensions: pruritus, swelling, impact on life activities, sleep problems, limits, and looks. Each item is scored on a 5-point Likert scale (1=not at all, 5=extremely) where participants indicate how troubled they are within each dimension. The individual items are summed to generate the overall CU-Q2oL score, which is then converted to a 0 to 100 scale; higher scores indicate greater QoL impairment.

Therapeutic methods are provided that result in a decrease in CU-Q2oL score from baseline. For example, administration of an IL-4R antagonist to a subject in need thereof causes a decrease in CU-Q2oL score from baseline of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R, and comprises the heavy chain and light chain (Complementarity Determining Region) CDR sequences from the Heavy Chain Variable Region (HCVR) and Light Chain Variable Region (LCVR) of SEQ ID NOs: 1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′) 2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}—CHI-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (Xi) V_{L}-C_{H}1-C_{H}2; (xii) V_{L}-CHI-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a Kp of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the disclosure.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “KD” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24:307-331, herein incorporated by reference.) Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild-type protein and a mutein thereof. (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the disclosure to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully-human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is dupilumab, which comprises the HCVR/LCVR amino acid sequence pair of SEQ ID NOs: 1 and 2.

In certain embodiments, the antibody sequence is dupilumab, which comprises the heavy chain/light chain amino acid sequence pair of SEQ ID NOs: 9 and 10.

Dupilumab HCVR amino acid sequence:
(SEQ ID NO: 1)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDRLSITIRP
RYYGLDVWGQGTTVTVS.
Dupilumab LCVR amino acid sequence:
(SEQ ID NO: 2)
DIVMTQSPLSLPVTPGEPASISCRSSQSLLYSIGYNYLDWYLQKSGQSPQLLIY
LGSNRASGVPDRFSGSGSGTDFTLKISRVEAEDVGFYYCMQALQTPYTFGQGTKLEI
K.
Dupilumab HCDR1 amino acid sequence:
(SEQ ID NO: 3)
GFTFRDYA.
Dupilumab HCDR2 amino acid sequence:
(SEQ ID NO: 4)
ISGSGGNT.
Dupilumab HCDR3 amino acid sequence:
(SEQ ID NO: 5)
AKDRLSITIRPRYYGL.
Dupilumab LCDRI amino acid sequence:
(SEQ ID NO: 6)
QSLLYSIGYNY.
Dupilumab LCDR2 amino acid sequence:
(SEQ ID NO: 7)
LGS.
Dupilumab LCDR3 amino acid sequence:
(SEQ ID NO: 8)
MQALQTPYT.
Dupilumab HC amino acid sequence:
(SEQ ID NO: 9)
EVQLVESGGGLEQPGGSLRLSCAGSGFTFRDYAMTWVRQAPGKGLEWVSS
ISGSGGNTYYADSVKGRFTISRDNSKNTLYLQMNSLRAEDTAVYYCAKDRLSITIRP
RYYGLDVWGQGTTVTVSSASTKGPSVFPLAPCSRSTSESTAALGCLVKDYFPEPVTV
SWNSGALTSGVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVVDVSQEDPEVQ
FNWYVDGVEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHYTQKSLS
LSLG
(amino acids 1-124 = HCVR; amino acids 125-451 = HC constant).
Dupilumab LC amino acid sequence:
(SEQ ID NO: 10)
DIVMTQSPLSLPVTPGEPASISCRSSQSLLYSIGYNYLDWYLQKSGQSPQLLIY
LGSNRASGVPDRFSGSGSGTDFTLKISRVEAEDVGFYYCMQALQTPYTFGQGTKLEI
KRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
(amino acids 1-112 = LCVR; amino acids 112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof of the disclosure comprises a LCVR/HCVR sequence pair of SCB-VL-44/SCB-VH-92.

In certain embodiments, an antibody or antigen-binding fragment thereof of the disclosure comprises a LCVR/HCVR sequence pair of SCB-VL-54/SCB-VH-92.

In certain embodiments, an antibody or antigen-binding fragment thereof of the disclosure comprises a LCVR/HCVR sequence pair of SCB-VL-55/SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. Pat. No. 10,774,141, incorporated herein by reference in its entirety for all purposes.

TABLE 2

| Sequence ID | Sequence |
|---|---|
| SCB-VL-39 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYGSSPP |
| | WTFGQGTKVEIK(SEQ ID NO: 11) |
| SCB-VL-40 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYGSSPP |
| | WTFGQGTKVEIK(SEQ ID NO: 12) |
| SCB-VL-41 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYGSSPP |
| | WTFGQGTKVEIK(SEQ ID NO: 13) |
| SCB-VL-42 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYGSSPP |
| | WTFGQGTKVEIK(SEQ ID NO: 14) |
| SCB-VL-43 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYGSSPP |
| | WTFGQGTKVEIK(SEQ ID NO: 15) |
| SCB-VL-44 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYGSSPP |
| | WTFGQGTKVEIK(SEQ ID NO: 16) |
| SCB-VL-45 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSPP |
| | WTFGQGTKVEIK(SEQ ID NO: 17) |
| SCB-VL-46 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSAG |
| | WTFGQGTKVEIK(SEQ ID NO: 18) |
| SCB-VL-47 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYDHSAG |
| | WTFGQGTKVEIK(SEQ ID NO: 19) |
| SCB-VL-48 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSPP |
| | WTFGQGTKVEIK(SEQ ID NO: 20) |
| SCB-VL-49 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSPP |
| | WTFGQGTKVEIK(SEQ ID NO: 21) |
| SCB-VL-50 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSPP |
| | WTFGQGTKVEIK(SEQ ID NO: 22) |
| SCB-VL-51 | EIVLTQSPGTLSLSPGERATLSCRASQSVSSSYLAWYQQKPGQAPRLL |
| | IYGASSRAPGIPDRFSGSGSGTDFTLTISRLEPEDFAVYYCQQYDHSA |
| | GWTFGQGTKVEIK(SEQ ID NO: 23) |
| SCB-VL-52 | EIVLTQSPGTLSLSPGERATLSCRASQSVSNSYLAWYQQKPGQAPRLL |
| | IFGASSRAPGIPDRFSGSGSGTDFTLTISRLEPEDFAVYYCQQYDHSAG |
| | WTFGQGTKVEIK(SEQ ID NO: 24) |
| SCB-VL-53 | EIVLTQSPGTLSLSPGERATLSCRASQSVSNSYLAWYQQKPGQAPRLL |
| | IYGASSRATGIPDRFSGSGSGTDFTLTISRLEPEDFAVYYCQQYDHSA |
| | GWTFGQGTKVEIK(SEQ ID NO: 25) |
| SCB-VL-54 | EIVLTQSPGTLSLSPGERATLSCRASQSVSSSYLAWYQQKPGQAPRLL |
| | IFGASSRAPGIPDRFSGSGSGTDFTLTISRLEPEDFAVYYCQQYDHSAG |
| | WTFGQGTKVEIK(SEQ ID NO: 26) |
| SCB-VL-55 | EIVLTQSPGTLSLSPGERATLSCRASQSVSSSYLAWYQQKPGQAPRLL |
| | IYGASSRATGIPDRFSGSGSGTDFTLTISRLEPEDFAVYYCQQYDHSA |
| | GWTFGQGTKVEIK(SEQ ID NO: 27) |
| SCB-VL-56 | EIVLTQSPGTLSLSPGERATLSCRASQSVSNSYLAWYQQKPGQAPRLL |
| | IFGASSRATGIPDRFSGSGSGTDFTLTISRLEPEDFAVYYCQQYDHSAG |
| | WTFGQGTKVEIK(SEQ ID NO: 28) |
| SCB-VL-57 | EIVLTQSPGTLSLSPGERATLSCRASQSVSSSYLAWYQQKPGQAPRLL |
| | IFGASSRATGIPDRFSGSGSGTDFTLTISRLEPEDFAVYYCQQYGSSPP |
| | WTFGQGTKVEIK(SEQ ID NO: 29) |
| SCB-VL-58 | EIVLTQSPGTLSLSPGERATLSCRASQSVSNSYLAWYQQKPGQAPRLL |
| | IYGASSRAPGIPDRFSGSGSGTDFTLTISRLEPEDFAVYYCQQYDHSA |
| | GWTFGQGTKVEIK(SEQ ID NO: 30) |
| SCB-VH-59 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 31) |
| SCB-VH-60 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 32) |
| SCB-VH-61 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 33) |
| SCB-VH-62 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFDYWGQGTLVTVSS(SEQ ID NO: 34) |
| SCB-VH-63 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFDYWGQGTLVTVSS(SEQ ID NO: 35) |
| SCB-VH-64 | EVQLVESGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 36) |
| SCB-VH-65 | EVQLVESGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 37) |
| SCB-VH-66 | EVQLVQSGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 38) |
| SCB-VH-67 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS(SEQ ID NO: 39) |
| SCB-VH-68 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS(SEQ ID NO: 40) |
| SCB-VH-69 | EVQLVESGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS(SEQ ID NO: 41) |
| SCB-VH-70 | EVQLVQSGGGLVQPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS(SEQ ID NO: 42) |
| SCB-VH-71 | EVQLVQSGGGLVHPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS(SEQ ID NO: 43) |
| SCB-VH-72 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS(SEQ ID NO: 44) |
| SCB-VH-73 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS(SEQ ID NO: 45) |
| SCB-VH-74 | EVQLVQSGGGLVHPGRSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 46) |
| SCB-VH-75 | EVQLVQSGGGLVHPGGSLRLTCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 47) |
| SCB-VH-76 | EVQLVQSGGGLVHPGGSLRLSCAGSGFTFSRNAMHWVRQAPGKGL |
| | EWVSGIGTGGATNYADSVKGRFTISRDNAKNSLYLQMNSLRAEDMA |
| | VYYCARGRYYFDYWGQGTLVTVSS(SEQ ID NO: 48) |
| SCB-VH-77 | EVQLVQSGGGLVHPGGSLRLSCAGSGFTFSRNAMFWVRQAPGEGLE |
| | WVSGIGTGGATNYADSVKGRFTISRDNAKNSLYLQMNSLRAEDMA |
| | VYYCARGRYYFDYWGQGTLVTVSS(SEQ ID NO: 49) |
| SCB-VH-78 | EVQLVQSGGGLVHPGGSLRLSCAGSGFTFSRNAMFWVRQAPGKGLE |
| | WVSGIGTGGATNYADSVKGRFTISRDEAKNSLYLQMNSLRAEDMAV |
| | YYCARGRYYFDYWGQGTLVTVSS(SEQ ID NO: 50) |
| SCB-VH-79 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAGDMA |
| | VYYCARGRYYFDYWGQGTLVTVSS(SEQ ID NO: 51) |
| SCB-VH-80 | EVQLVQSGGGLVHPGGSLRLSCAGSGFTFDDYAMFWVRQAPGKGL |
| | EWVSGIGTGGATNYADSVKGRFTISRDNAKNSLYLQMNSLRAEDMA |
| | VYYCARGRYYFDYWGQGTLVTVSS(SEQ ID NO: 52) |
| SCB-VH-81 | EVQLVQSGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 53) |
| SCB-VH-82 | EVQLVESGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 54) |
| SCB-VH-83 | EVQLVESGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 55) |
| SCB-VH-84 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDTAV |
| | YYCARGRYYFPWWGQGTLVTVSS(SEQ ID NO: 56) |
| SCB-VH-85 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDMAV |
| | YYCARGRYYFPWWGQGTLVTVSS(SEQ ID NO: 57) |
| SCB-VH-86 | EVQLVQSGGGLVHPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 58) |
| SCB-VH-87 | EVQLVQSGGGLVQPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 59) |
| SCB-VH-88 | EVQLVESGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 60) |
| SCB-VH-89 | EVQLVQSGGGLVHPGGSLRLSCAGSGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 61) |
| SCB-VH-90 | EVQLVESGGGLVQPGGSLRLSCAASGFTFSRNAMFWVRQAPGKGLE |
| | WVSGIGTGGATNYADSVKGRFTISRDNAKNSLYLQMNSLRAEDMA |
| | VYYCARGRYYFPWWGQGTLVTVSS(SEQ ID NO: 62) |
| SCB-VH-91 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFDYWGQGTLVTVSS(SEQ ID NO: 63) |
| SCB-VH-92 | EVQLVQSGGGLVHPGGSLRLSCAGSGFTFSRNAMFWVRQAPGKGLE |
| | WVSGIGTGGATNYADSVKGRFTISRDNAKNSLYLQMNSLRAEDMA |
| | VYYCARGRYYFDYWGQGTLVTVSS(SEQ ID NO: 64) |
| SCB-VH-93 | EVQLVESGGGLVQPGGSLRLSCAASGFTFSRNAMFWVRQAPGKGLE |
| | WVSGIGTGGATSYADSVKGRFTISRDNAKNSLYLQMNSLRAEDTAV |
| | YYCARGRYYFPWWGQGTLVTVSS(SEQ ID NO: 65) |
| SCB-92- | RNAMF(SEQ ID NO: 66) |
| HCDR1 | |
| SCB-92- | GIGTGGATSYADSVKG(SEQ ID NO: 67) |
| HCDR3 | |
| SCB-92- | GRYYFDY(SEQ ID NO: 68) |
| HCDR3 | |
| SCB-55- | RASQSVSSSYLA(SEQ ID NO: 69) |
| LCDR1 | |
| SCB-55- | GASSRAT(SEQ ID NO: 70) |
| LCDR2 | |
| SCB-55- | QQYDHSAGWT(SEQ ID NO: 71) |
| LCDR3 | |
| SCB-54- | GASSRAP(SEQ ID NO: 72) |
| LCDR2 | |
| SCB-44- | QQYGSSPPWT(SEQ ID NO: 73) |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof of the disclosure comprises a LCVR/HCVR sequence pair of MEDI-37GL-VL/MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. Pat. No. 8,877,189, incorporated herein by reference in its entirety for all purposes.

TABLE 2

| Sequence ID | Sequence |
|---|---|
| MEDI-1-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLDYWGKGTLVTVSS(SEQ ID NO: 74) |
| MEDI-1-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SLSANYVFGTGTKLTVL(SEQ ID NO: 75) |
| MEDI-2-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-2-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SQPPNPLFGTGTKLTVL(SEQ ID NO: 77) |
| MEDI-3-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKLLKNPWGKGTLVTVSS(SEQ ID NO: 78) |
| MEDI-3-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWFG |
| | TPASNYVFGTGTKLTVL(SEQ ID NO: 79) |
| MEDI-4-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-4-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL(SEQ ID NO: 80) |
| MEDI-5-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS(SEQ ID NO: 81) |
| MEDI-5-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL(SEQ ID NO: 80) |
| MEDI-6-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-6-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL(SEQ ID NO: 83) |
| MEDI-7-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS(SEQ ID NO: 84) |
| MEDI-7-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SSPPQPIFGTGTKLTVL(SEQ ID NO: 80) |
| MEDI-8-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQYWGKGTLVTVSS(SEQ ID NO: 84) |
| MEDI-8-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL(SEQ ID NO: 83) |
| MEDI-9-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-9-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL(SEQ ID NO: 85) |
| MEDI-10-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS(SEQ ID NO: 81) |
| MEDI-10-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVLTPIFGTGTKLTVL(SEQ ID NO: 86) |
| MEDI-11-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS(SEQ ID NO: 87) |
| MEDI-11-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPSMIPLFGTGTKLTVL(SEQ ID NO: 88) |
| MEDI-12-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWFYDWGKGTLVTVSS(SEQ ID NO: 87) |
| MEDI-12-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTMYPLFGTGTKLTVL(SEQ ID NO: 85) |
| MEDI-13-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYDWGKGTLVTVSS(SEQ ID NO: 81) |
| MEDI-13-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTLQPLFGTGTKLTVL(SEQ ID NO: 89) |
| MEDI-14-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-14-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPPTKPLFGTGTKLTVL(SEQ ID NO: 90) |
| MEDI-15-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-15-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STHRHPLFGTGTKLTVL(SEQ ID NO: 91) |
| MEDI-16-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWLYNWGKGTLVTVSS(SEQ ID NO: 76) |
| MEDI-16-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL(SEQ ID NO: 83) |
| MEDI-17-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS(SEQ ID NO: 92) |
| MEDI-17-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVDRPIFGTGTKLTVL(SEQ ID NO: 93) |
| MEDI-18-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS(SEQ ID NO: 92) |
| MEDI-18-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTPMPVFGTGTKLTVL(SEQ ID NO: 94) |
| MEDI-19-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKWWWQHWGKGTLVTVSS(SEQ ID NO: 92) |
| MEDI-19-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STTYHPIFGTGTKLTVL(SEQ ID NO: 83) |
| MEDI-20-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-20-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STVWEWPFGTGTKLTVL(SEQ ID NO: 95) |
| MEDI-21-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS(SEQ ID NO: 96) |
| MEDI-21-VL | QSVLTQPPSVSAAPGQKVTISCSGGSSNIGNSYVSWYQQLPGTAPKL |
| | LIYDNNKRPSGIPDRFSGSKSGTSATLAITGLQTGDEAVYFCGTWDT |
| | STVWEWPFGTGTKLTVL(SEQ ID NO: 97) |
| MEDI-22-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-22-VL | QPVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 98) |
| MEDI-23-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 99) |
| MEDI-23-VL | QSVLTQPPSVSAAPGQKVTISCSGGSSNIGNNYVSWYQQLPGTAPKL |
| | LIYDNNKRPPGIPDRFSGSKSGTSATLAITGLQTGDEADYYCGTWDT |
| | STVWEWPFGTGTKLTVL(SEQ ID NO: 100) |
| MEDI-24-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS(SEQ ID NO: 101) |
| MEDI-24-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 102) |
| MEDI-25-VH | QVQLVQSGAEVKKPGASVKVSCKASGYAFTSYYMHWARQAPGQG |
| | LEWMGIINPRGGSASYAQKFQGRVSMTRDTSTSTVYMELSSLRSED |
| | TAVYYCARGKYWMYDWGKGTLVTVSS(SEQ ID NO: 103) |
| MEDI-25-VL | QSVLTQPPSVSAAPGQKVTISCSGGSSNIGNSYVSWYQQLPGTAPKL |
| | LIYDNNKRPSGIPDRFSGSKSGTTATLAITGLQTGDEADYYCGTWVT |
| | STVWEWPFGTGTKLTVL(SEQ ID NO: 104) |
| MEDI-26-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-26-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 102) |
| MEDI-27-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTQVTVSS(SEQ ID NO: 105) |
| MEDI-27-VL | QSVLTQPPLVSAAPGQKVTISCSGGSSNIGNSYVSWYQRLPGTAPKL |
| | LIYDNNKRPSGIPDRFSGSKSGTSATLAITGLQTGDEADYYCGTWDT |
| | STVWEWPFGTGTKLTVL(SEQ ID NO: 106) |
| MEDI-28-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGNGTLVTVSS(SEQ ID NO: 107) |
| MEDI-28-VL | LPVLTQPPSVSAAPGQKVTISCSGGSSSIGNSYVSWYQQLPGAAPKL |
| | LIYDNNKRPSGIPDRFSGFRSGTSATLAITGLQTGDEADYYCGTWDT |
| | SPVWEWPFGTGTKLTVL(SEQ ID NO: 108) |
| MEDI-29-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTRVTVSS(SEQ ID NO: 109) |
| MEDI-29-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL(SEQ ID NO: 110) |
| MEDI-30-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-30-VL | QSVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STVWEWPFGTGTKLTVL(SEQ ID NO: 111) |
| MEDI-31-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-31-VL | QSVLTQPPSVSAAPGQKVTISCSGGSSSIGNSYVSWYQQLPGTAPKL |
| | LIYDNNKRPSGIPDRFSGSKSGTSATLAITGLQTGDEADYYCGTWAT |
| | SPVWEWPFGTGTKLTVL(SEQ ID NO: 112) |
| MEDI-32-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-32-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STAWEWPFGTGTKLTVL(SEQ ID NO: 113) |
| MEDI-33-VH | QVQLVQSGAEEKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 114) |
| MEDI-33-VL | QSALTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 115) |
| MEDI-34-VH | QVQLVQSGAEVKKPGASVKVSCKASGYAFTSYYMHWARQAPGQG |
| | LEWMGIINPSGGSTSYAQKFQGRVSMTRDTSTSTVYMELSSLRSEDT |
| | AVYYCARGKYWMYDWGKGTLVTVSS(SEQ ID NO: 116) |
| MEDI-34-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 102) |
| MEDI-35-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-35-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | SPVWEWPFGTGTKLTVL(SEQ ID NO: 110) |
| MEDI-36-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS(SEQ ID NO: 96) |
| MEDI-36-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL(SEQ ID NO: 117) |
| MEDI-37-VH | QVQLVQSGAEVKKPGASVKVSCKASGYAFTSYYMHWARQAPGQG |
| | LEWMGIINPRGGSTSYAQKFQGRVAMTRDTSTSTVYMELSSLRPED |
| | TAVYYCARGKYWMYDWGKGTLVTVSS(SEQ ID NO: 118) |
| MEDI-37-VL | QSVLTQPPSVSAAPGQKVTISCSGGGSSIGNSYVSWYQQLPGTAPKL |
| | LIYDNNKRPSGVPDRFSGSKSGTSATLAITGLQTGDEADYYCGTWD |
| | TSPVWEWPFGTGTKLTVL(SEQ ID NO: 119) |
| MEDI-38-VH | QVQLVQSGAEVKKPGASVKVSCKASGYAFTSYYMHWARQAPGQG |
| | LEWMGIINPSGGSASYAQKFQGRVTMTRDTSTSTVYMELSSLRSED |
| | TAVYYCARGKYWMYDWGKGTLVTVSS(SEQ ID NO: 96) |
| MEDI-38-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYFCGTWDT |
| | STVWEWPFGTGTKLTVL(SEQ ID NO: 102) |
| MEDI-39-VH | QVQLVQSGAEVKKPGASVKVSCKASGYAFTSYYMHWARQAPGQG |
| | LEWMGIINPRGGSTSYAQKFQGRVTMTRDTSTSTVYMELSSLRSED |
| | TAVYYCARGKYWMYDWGKGTLVTVSS(SEQ ID NO: 101) |
| MEDI-39-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDT |
| | STAWEWPFGTGTKLTVL(SEQ ID NO: 120) |
| MEDI-40-VH | QVQLVQSGAEVKKPGASVKVSCKASGYAFTSYYMHWARQAPGQG |
| | LEWMGIINPSGGSTSYAQKFQGRVTMTRDTSTSTVYMELSSLRSED |
| | TAVYYCARGKYWMYDWGKGTLVTVSS(SEQ ID NO: 82) |
| MEDI-40-VL | QSVLTQPPSVSAAPGQKVTISCSGGSSNIGNSYVSWYQQLPGTAPKL |
| | LIYDNNKRPSGIPDRFSGSKSGTSATLAITGLQTGDEADYYCGTWDS |
| | STVWEWPFGTGTKLTVL(SEQ ID NO: 117) |
| MEDI-41-VH | QVQLVQSGAEVRKPGASVKVSCKASGYAFTSYYMHWARQAPGQG |
| | LEWMGIINPSGGSTSYAQKFQGRVTMTRDTSTSTVYMELSSLRPED |
| | TAVYYCARGKYWMYDWGKGTLVTVSG(SEQ ID NO: 121) |
| MEDI-41-VL | QSVLTQPPSVSAAPGQKVTISCSGGSTNIGNSYVSWYQRLPGTAPKL |
| | LIYDNNKRPPGIPDRFSGSKSGTSATLAITGLQTGDEADYYCGTWDT |
| | STVWEWPFGTGTKLTVL(SEQ ID NO: 122) |
| MEDI-42-VH | QVQLVQSGAEVKKPGASVKVSCKASGYAFTSYYMHWARQAPGQG |
| | LEWVGIINPSGGSTSYAQKFQGRVTMTRDTSTSTVYMELSSLRSGDT |
| | AVYYCARGKYWMYDWGKGTLVTVSS(SEQ ID NO: 123) |
| MEDI-42-VL | QAVLTQPPSVSAAPGQKVTISCSGGSSNIGNSYVSWYQRLPGAAPKL |
| | LIYDNNKRPSGIPDRFSGSKSGTSATLAITGLQTGDEADYYCGTWDT |
| | STGWEWPFGTGTKLTVL(SEQ ID NO: 124) |
| MEDI-37GL- | QVQLVQSGAEVKKPGASVKVSCKASGYAFTSYYMHWVRQAPGQG |
| VH | LEWMGIINPRGGSTSYAQKFQGRVTMTRDTSTSTVYMELSSLRSED |
| | TAVYYCARGKYWMYDWGKGTLVTVSS(SEQ ID NO: 125) |
| MEDI-37GL- | QSVLTQPPSVSAAPGQKVTISCSGGGSSIGNSYVSWYQQLPGTAPKL |
| VL | LIYDNNKRPSGIPDRFSGSKSGTSATLGITGLQTGDEADYYCGTWDT |
| | SPVWEWPFGTGTKLTVL(SEQ ID NO: 126) |
| MEDI-37GL- | SYYMH(SEQ ID NO: 127) |
| HCDR1 | |
| MEDI-37GL- | IINPRGGSTSYAQKFQG(SEQ ID NO: 128) |
| HCDR2 | |
| MEDI-37GL- | GKYWMYD(SEQ ID NO: 129) |
| HCDR3 | |
| MEDI-37GL- | SGGGSSIGNSYVS(SEQ ID NO: 130) |
| LCDR1 | |
| MEDI-37GL- | DNNKRPS(SEQ ID NO: 131) |
| LCDR2 | |
| MEDI-37GL- | GTWDTSPVWEWP(SEQ ID NO: 132) |
| LCDR3 | |

In certain embodiments, an antibody or antigen-binding fragment thereof of the disclosure comprises a LCVR/HCVR sequence pair of AJOU-90-VL/AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9:7772. 2019), incorporated herein by reference in their entireties for all purposes.

TABLE 3

| Sequence ID | Sequence |
|---|---|
| AJOU-1-VH | EVQLLESGGGLVQPGGSLRLSCAVSGFTFSNYAMSWVRQAPGKGL |
| | EWVSAISSGGGNIYYADSVKGRFTISRDNSKNTLYLQMNSLRAEDT |
| | AVYYCAKLRRYFDYWGQGTLVTVSS(SEQ ID NO: 133) |
| AJOU-2-VH | EVQLLESGGGLVQPGGSLRLSCAASGFTFSDYAMSWVRQAPGKGL |
| | EWVSAISSGGSSIYYADSVKGRFTISRDNSKNTLHLQMNSLRAEDT |
| | AVYYCARGPQRSATAVFDYWGQGTLVTVSS(SEQ ID NO: 134) |
| AJOU-3-VH | EVQLLESGGGLVQPGGSLRLSCAASGFTFSNYAMSWVRQAPGKGL |
| | EWVSWISPNSGNIYYADSVKGRFTISRDNSKNTLYLQMNSLRAEDT |
| | AVYYCARRPLSAAWSHSSYYNAMDVWGQGTLVTVSS(SEQ ID |
| | NO: 135) |
| AJOU-4-VH | EVQLLESGGGLVQPGGSLRLSCAASGFTFSGYAMSWVRQAPGKGL |
| | EWVSLISHSGSNTYYADSVKGRFTISRDNSKNTLYLQMNSLRAEDT |
| | AVYYCARPHRAFDYWGQGTLVTVSS(SEQ ID NO: 136) |
| AJOU-5-VH | EVQLLESGGGLVQPGGSLRLSCAASGFTFSNYAMSWVRQAPGKGL |
| | EWVSGISHGSGSIYYADSVKGRFTISRDNSKNTLYLQMNSLRAEDT |
| | AVYYCARPHRAFDYWGQGTLVTVSS(SEQ ID NO: 137) |
| AJOU-6-VH | EVQLLESGGGLVQPGGSLRLSCAASGFTFSNYAMSWVRQAPGKGL |
| | EWVSGISHGNGSIYYADSVKGRFTISRDNSKNTLYLQMNSLRAEDT |
| | AVYYCAKTGRHFDYWGQGTLVTVSS(SEQ ID NO: 138) |
| AJOU-7-VH | EVQLLESGGGLVQPGGSLRLSCAASGFTFSNYAMSWVRQAPGKGL |
| | EWVSSISPSGSSIYYADSVKGRFTISRDNSKNTLYLQMNSLRAEDTA |
| | VYYCARSYRAFDYWGQGTLVTVSS(SEQ ID NO: 139) |
| AJOU-8-VH | EVQLLESGGGLVQPGGSLRLSCAASGFTFSNYAMSWVRQAPGKGL |
| | EWVSAISPSGGSIYYADSVKGRFTISRDNSKNTLYLQMNSLRAEDT |
| | AVYYCARAKRAFDYWGQGTLVTVSS(SEQ ID NO: 140) |
| AJOU-9-VH | EVQLLESGGGLVQPGGSLRLSCAASGFTFSNYAMSWVRQAPGKGL |
| | EWVSAISPGSGSTYYADSVKGRFTISRDNSKNTLYLQMNSLRAEDT |
| | AVYYCAKFRRHFDYWGQGTLVTVSS(SEQ ID NO: 141) |
| AJOU-10-VH | EVQLLESGGGLVQPGGSLRLSCAASGFTFSNYAMSWVRQAPGKGL |
| | EWVSAISSGGGNIYYADSVKGRFTISRDNSKNTLYLQMNSLRAEDT |
| | AVYYCARVHRAFDYWGQGTLVTVSS(SEQ ID NO: 142) |
| AJOU-69-VH | EVQLLESGGGLVQPGGSLRLSCAASGFTFSNYAMSWVRQAPGKGL |
| | EWVSAITSSGRSIYYADSVKGRFTISRDNSKNTLYLQMNSLRAEDT |
| | AVYYCARVHRAFDYWGQGTLVTVSS(SEQ ID NO: 143) |
| AJOU-70-VH | EVQLLESGGGLVQPGGSLRLSCAASGFTFSNYAMSWVRQAPGKGL |
| | EWVSAITSSGANIYYADSVKGRFTISRDNSKNTLYLQMNSLRAEDT |
| | AVYYCARVHRAFDYWGQGTLVTVSS(SEQ ID NO: 144) |
| AJOU-71-VH | EVQLLESGGGLVQPGGSLRLSCAASGFTFSNYAMSWVRQAPGKGL |
| | EWVSAITSSGGNIYYADSVKGRFTISRDNSKNTLYLQMNSLRAEDT |
| | AVYYCARVHRAFDYWGQGTLVTVSS(SEQ ID NO: 145) |
| AJOU-72-VH | EVQLLESGGGLVQPGGSLRLSCAASGFTFSNYAMSWVRQAPGKGL |
| | EWVSAITAGGGSIYYADSVKGRFTISRDNSKNTLYLQMNSLRAEDT |
| | AVYYCARVHRAFDYWGQGTLVTVSS(SEQ ID NO: 146) |
| AJOU-83-VH | EVQLLESGGGLVQPGGSLRLSCAASGFTFSRHAMAWVRQAPGKGL |
| | EWVSAITSSGRSIYYADSVKGRFTISRDNSKNTLYLQMNSLRAEDT |
| | AVYYCARVHRAFDYWGQGTLVTVSS(SEQ ID NO: 147) |
| AJOU-33-VL | QSVLTQPPSASGTPGQRVTISCSGSSSNIGNNYVNWYQQLPGTAPK |
| | LLIYDNSHRPSGVPDRFSGSKSGTSASLAISGLRSEDEADYYCGTW |
| | DASLSAYVFGGGTKLTVL(SEQ ID NO: 148) |
| AJOU-34-VL | QSVLTQPPSASGTPGQRVTISCSGSSSNIGNNNVSWYQQLPGTAPKL |
| | LIYANSKRPSGVPDRFSGSKSGTSASLAISGLRSEDEADYYCGSWD |
| | DSLSAYVFGGGTKLTVL(SEQ ID NO: 149) |
| AJOU-35-VL | QSVLTQPPSAPGTPGQRVTISCTGSSSNIGSNSVNWYQQLPGTAPKL |
| | LIYDDSHRPSGVPDRFSGSKSGTSASLAISGLRSEDEADYYCDAWD |
| | SSLSAYVFGGGTKLTVL(SEQ ID NO: 150) |
| AJOU-36-VL | QSVLTQPPSASGTPGQRVTLSCTGSSSNIGSNYVSWYQQLPGTAPK |
| | LLIYADSQRPSGVPDRFSGSKSGTSASLAISGLRSEDEADYYCGTW |
| | DDSLSGYVFGGGTKLTVL(SEQ ID NO: 151) |
| AJOU-37-VL | QSVLTQPPSASGTPGQRVTISCSSSSSNIGSNYVSWYQQLPGTAPKL |
| | LIYSDSHRPSGVPDRFSGSKSGTSASLAISGLRSEDEADYYCGSWDY |
| | SLSAYVFGGGTKLTVL(SEQ ID NO: 152) |
| AJOU-38-VL | QSVLTQPPSASGTPGQRVTISCTGSSSNIGNNTVSWYQQLPGTAPKL |
| | LIYDNSHRPSGVPDRFSGSKSGTSASLAISGLQSEDEADYYCGSWD |
| | YSLSAYVFGGGTKLTVL(SEQ ID NO: 153) |
| AJOU-39-VL | QSVLTQPPSASGTPGQRVTISCTGSSSNIGNNDVNWYQQLPGTAPK |
| | LLIYYDSQRPSGVPDRFSGSKSGTSASLAISGLRSEDEADYYCATW |
| | DASLSAYVFGGGTKLTVL(SEQ ID NO: 154) |
| AJOU-40-VL | QSVLTQPPSASGTPGQRVTISCSGSSSNIGSNAVNWYQQLPGTAPKL |
| | LIYYDNQRPSGVPDRFSGSKSGTSASLAISGLRSEDEADYYCGTWD |
| | DSLNGYVFGGGTKLTVL(SEQ ID NO: 155) |
| AJOU-41-VL | QSVLTQPPSASGTPGQRVTISCSGSSSNIGNNAVTWYQQLPGTAPK |
| | LLIYDDSHRPSGVPDRFSGSKSGTSASLAISGLRSEDEADYYCGSW |
| | DYSLSAYVFGGGTKLTVL(SEQ ID NO: 156) |
| AJOU-42-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL(SEQ ID NO: 157) |
| AJOU-77-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL(SEQ ID NO: 157) |
| AJOU-78-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLRGYVLGGGTKLTVL(SEQ ID NO: 158) |
| AJOU-79-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGYWD |
| | YSLSGYVLGGGTKLTVL(SEQ ID NO: 159) |
| AJOU-80-VL | QSVLTQPPSASGTPGQRVTISCSGSSSNIGSNTFNWYQQLPGTAPKL |
| | LIYADSHRPSGVPDRFSGSKSGTSASLAISGLRSEDEADYYCGTWD |
| | YSLSGYVLGGGTKLTVL(SEQ ID NO: 157) |
| AJOU-86-VL | QSVLTQPPSASGTPGQRVTISCSGSSANSRTDGFNWYQQLPGTAPK |
| | LLIYADSHRPSGVPDRFSGSKSGTSASLAISGLRSEDEADYYCGTW |
| | DYSLSGYVLGGGTKLTVLG(SEQ ID NO: 160) |
| AJOU-87-VL | QSVLTQPPSASGTPGQRVTISCSGSAQFGSRDNFNWYQQLPGTAPK |
| | LLIYADSHRPSGVPDRFSGSKSGTSASLAISGLRSEDEADYYCGTW |
| | DYSLSGYVLGGGTKLTVLG(SEQ ID NO: 161) |
| AJOU-88-VL | QSVLTQPPSASGTPGQRVTISCSGSTKQMHNYQFNWYQQLPGTAP |
| | KLLIYADSHRPSGVPDRFSGSKSGTSASLAISGLRSEDEADYYCGT |
| | WDYSLSGYVLGGGTKLTVLG(SEQ ID NO: 162) |
| AJOU-89-VL | QSVLTQPPSASGTPGQRVTISCSGSLLRGENLQFNWYQQLPGTAPK |
| | LLIYADSHRPSGVPDRFSGSKSGTSASLAISGLRSEDEADYYCGTW |
| | DYSLSGYVLGGGTKLTVLG(SEQ ID NO: 163) |
| AJOU-90-VL | QSVLTQPPSASGTPGQRVTISCSGSPLFPDSGSFNWYQQLPGTAPKL |
| | LIYADSHRPSGVPDRFSGSKSGTSASLAISGLRSEDEADYYCGTWD |
| | YSLSGYVLGGGTKLTVLG(SEQ ID NO: 164) |
| AJOU-91-VL | QSVLTQPPSASGTPGQRVTISCSGSAALDLSPSFNWYQQLPGTAPKL |
| | LIYADSHRPSGVPDRFSGSKSGTSASLAISGLRSEDEADYYCGTWD |
| | YSLSGYVLGGGTKLTVLG(SEQ ID NO: 165) |
| AJOU-84- | RHAMA(SEQ ID NO: 166) |
| HCDR1 | |
| AJOU-85- | AITSSGRSIYYADSVKG(SEQ ID NO: 167) |
| HCDR2 | |
| AJOU-32- | VHRAFDY(SEQ ID NO: 168) |
| HCDR3 | |
| AJOU-96- | SGSPLFPDSGSFN(SEQ ID NO: 169) |
| LCDR1 | |
| AJOU-60- | ADSHRPS(SEQ ID NO: 170) |
| LCDR2 | |
| AJOU-68- | GTWDYSLSGYV(SEQ ID NO: 171) |
| LCDR3 | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence ID | Sequence |
|---|---|
| REGN-VH-3 | QVQLVQSGAEVKKPGASVKVSCKASGYTFTNYGISWVRQAPGQG |
| | LEWMGWISVYNGKTNYAQKLQGRVTMTTDTSTTTAYMEMRSLR |
| | SDDTAVYYCARGSGYDLDYWGQGTLVSVSS(SEQ ID NO: 172) |
| REGN-VH-19 | EVQLVESGGGLVQPGGSLRLSCAASGFTFSSFWMTWVRQAPGKG |
| | LEWVANIKQDGSEKYYVDSVKGRFTISRDNAKNSLYLQMNSLRA |
| | EDTAVYYCARDPGRTMVRGGIRYYYGMDVWGQGTTVTVSS |
| | (SEQ ID NO: 173) |
| REGN-VH-35 | EVKLAESGGGLVQPGGSLRLSCAASGFTFSSHWMNWVRQAPGKG |
| | LEWVANIKQDGSDKYYVDSVKGRFTISRDNAKNSLYLQLNSLIAE |
| | DTAVYYCARDRGVRPPRGAFDIWGQGTMVTVSS(SEQ ID NO: |
| | 174) |
| REGN-VH-51 | QVQLVQSGAEVKKPGASVKVSCKASGYTFNSYGISWVRQAPGQG |
| | LEWMGWIRTYNGNTNYAQKLQGRVTMTTDTSTSTAYMELRSLR |
| | SDDTAVYYCARDEARIVVAGTTPYYYGMDVWGQGTTVTVSS |
| | (SEQ ID NO: 175) |
| REGN-VH-67 | QVQLVESGGGLVQPGGSLRLSCAVSGFTISDHYMSWIRQAPGKGL |
| | EWISYISSSGSKIYYADSVKGRFTISRDNAKNSLFLQMNSLRAEDT |
| | AVYYCARTRQLVGDYWGQGTLVTVSS(SEQ ID NO: 176) |
| REGN-VH-83 | EVQLVESGGGLVQPGRSLRLSCAASGFTFDNYAMHWVRQAPGK |
| | GLEWVSGIRWNSGSIGYADSVKGRFTISRDNAKNSLYLQMNSLRA |
| | EDTALYYCAKEGGYSGYRPGPFFDYWGQGTLVTVSS(SEQ ID NO: |
| | 177) |
| REGN-VH-99 | QVQLVQSGAEVKKPGASVKVSCKASGYTFTNYGISWVRQAPGQG |
| | LEWMGWISVYNGHTNYAQKLQGRVTMTTDTSTSTAYMELRSLR |
| | SDDTAVYYCARGSGYDFDSWGQGTLVTVSS(SEQ ID NO: 178) |
| REGN-VH-115 | QVQLVQSGAEVKKPGASVKVSCKASRYTFTSYDINWVRQATGQG |
| | LEWMGWMNPNSGNTGYAQKFQGRVTMTRNTSTSTAYMELSSLR |
| | SEDTAVYYCARVRRFFDYWGQGTLVTVSS(SEQ ID NO: 179) |
| REGN-VH-147 | QVQLVQSGPEVKKPGASVKVSCKASGYTFTNYGISWVRQAPGQG |
| | LEWMGWISVYNGNINYAQKLQGRVTMTTDTSTSTAYMDLRSLRS |
| | DDTAVYYCARGSGYDFDYWGQGTLVTVSS(SEQ ID NO: 180) |
| REGN-VH-163 | QVQLVQSGAEVKKPGASVKVSCKDSAYTFNRYGISWVRQAPGQG |
| | LEWMGWISAYTGNTVYAQKLQGRVTMTTDNSTSTAYMELRSLR |
| | SDDTAVYYCARDKSIFGVVRGFDYWGQGTLVTVSS(SEQ ID NO: |
| | 181) |
| REGN-VL-11 | AIQMTQSPSSLSASVGDRVTITCRASQGIRNALGWYQQKPGKAPK |
| | LLIYAASSLQSGVPSRFSGSGSGTDFTLTFSSLQPEDFATYYCLQDF |
| | NYPYTFGQGTKLEIK(SEQ ID NO: 182) |
| REGN-VL-27 | DIQMTQSPSSVSASVGDRVTISCRASQGVSSWLAWYQQKPGNAP |
| | KLLISAASSIQSGVPSRFSGSGSGTDFTLTISSLQPEDFATYYCQQA |
| | NSFPLTFGGGTKVEIK(SEQ ID NO: 183) |
| REGN-VL-43 | DIQMTQSPSSVSASVGDRVTITCRASQGISSWLAWYQQKPGKAPK |
| | LLIYAASSFQSGVPSRFSGSGSGTDFTLTISSLQPEDFATYFCQQAN |
| | SFPLTFGGGTTVEIK(SEQ ID NO: 184) |
| REGN-VL-59 | DIQMTQSPSSVSASVGDRVTITCRASQDISIWLAWYQQSPGKAPKL |
| | LINVASRLQSGVPSRFSGSGSGTDFTLTINSLQPEDFVTYYCQQAN |
| | SFPITFGQGTRLATK(SEQ ID NO: 185) |
| REGN-VL-75 | DIQLTQSPSFLSASVGDRVTITCWASQGISSYLAWYQQKPGKAPKL |
| | LIFAASTLQSGVPSRFSGSGSGTEFTLTISSLQPEDFATYYCQQLNS |
| | YPLTFGGGTKVEIR(SEQ ID NO: 186) |
| REGN-VL-91 | EIVMTQSPATLSVSPGERATLSCRASQSVNYNLAWYQHKPGQAPR |
| | LLIYGASTRATGIPARFSGSGSGTEFTLTISSLQSEDFAVYYCQQYN |
| | NWPLTFGGGTKVEIK(SEQ ID NO: 187) |
| REGN-VL-107 | AIQMTQSSSSLSASVGDRVTITCRASQAIRNALGWYQQKPGKAPK |
| | VLIYAASSLQSGIPSRFSGSGSGTDFTLTISSLQPEDFATYYCLQDY |
| | DYPYTFGQGTKLEIK(SEQ ID NO: 188) |
| REGN-VL-123 | DIQLTQSPSFLSASVGDRVTITCWASQGIISYLAWYQQKPGKAPKL |
| | LIYAASTLHSGVPSRFSGSGSGTEFTLTISSLQPEDFATYYCHQLKS |
| | YPITFGQGTRLEIK(SEQ ID NO: 189) |
| REGN-VL-155 | AIQMTQSPSSLSASVGDRVTITCRASQDIRNALGWYQQKPGKAPK |
| | LLIYAASSLQSGVPSRFSGSASGTDFTLTISSLQPEDFAAYYCLQDY |
| | NYPYTFGQGTKLEIK(SEQ ID NO: 190) |
| REGN-VL-171 | EIVMTQSPVTLSLSPGERATLPCRASQSVSSSLAWYQQKAGQSPRL |
| | LIYGASTRATGIPARFSGSGSGTEFTLTISNLQSEDFAVYYCQQYN |
| | NWPLTFGGGTKVEIK(SEQ ID NO: 191) |

In certain embodiments, an anti-IL-4Rα antibody comprises: (i) an HCVR comprising the amino acid sequence STSA-C27-VH, STSA-C27-6-33-VH, STSA-C27-7-33-VH, STSA-C27-24-56-VH, STSA-C27-47-56-VH, STSA-C27-33-33-VH, STSA-C27-56-56-VH, STSA-C27-78-78-VH, STSA-C27-82-58-VH, STSA-C27-54-54-VH, STSA-C27-36-36-VH, STSA-C27-53-53-VH, STSA-C27-67-67-VH, STSA-C27-55-55-VH, STSA-C27-59-59-VH, STSA-C27-58-58-VH, STSA-C27-52-52-VH, or STSA-C27-Y2-Y2-VH; and (ii) an LCVR comprising the amino acid sequence of STSA-C27-VL, STSA-C27-6-33-VL, STSA-C27-7-33-VL, STSA-C27-24-56-VL, STSA-C27-47-56-VL, STSA-C27-33-33-VL, STSA-C27-56-56-VL, STSA-C27-78-78-VL, STSA-C27-82-58-VL, STSA-C27-54-54-VL, STSA-C27-36-36-VL, STSA-C27-53-53-VL, STSA-C27-67-67-VL, STSA-C27-55-55-VL, STSA-C27-59-59-VL, STSA-C27-58-58-VL, TSA-C27-52-52-VL, or SEQ ID NO:243.

The antibodies recited below in Table 5 are described in more detail in WO2022/052974, incorporated herein by reference in its entirety for all purposes.

TABLE 5

| EVQLLESGGGLVQPGGSLRLSCAASGFTLSSYAMHWVR | STSA-C27-VH |
|---|---|
| QAPGKGLEYVSGISSNGGSTYYANSVKGRFTISRDNPKN | |
| TLFLQMSSLRAEDTAVYYCVRVKVGYRGGMDVWGQG | |
| TTVTVSS(SEQ ID NO: 192) | |
| EVQLLESGGGLVQPGGSLRLSCAASGFTLSSYAMHWVR | STSA-C27-6-33-VH |
| QAPGKGLEYVSGISPSGSSTYYANSVKGRFTISRDNPKNT | |
| LFLQMSSLRAEDTAVYYCVRSKVRYRGGMDVWGQGTT | |
| VTVSS(SEQ ID NO: 193) | |
| EVQLLESGGGLVQPGGSLRLSCAASGFTLSSYAMHWVR | STSA-C27-7-33-VH |
| QAPGKGLEYVSGISPSGVSTYYANSVKGRFTISRDNPKN | |
| TLFLQMSSLRAEDTAVYYCVRVKVKYRGGMDVWGQG | |
| TTVTVSS(SEQ ID NO: 194) | |
| EVQLLESGGGLVQPGGSLRLSCAASGFTLSSYAMHWVR | STSA-C27-24-56- |
| QAPGKGLEYVSGISPTSGSTYYANSVKGRFTISRDNPKNT | VH |
| LFLQMSSLRAEDTAVYYCVRVKVRYRGGMDVWGQGTT | |
| VTVSS(SEQ ID NO: 195) | |
| EVQLLESGGGLVQPGGSLRLSCAASGFTLSSYAMHWVR | STSA-C27-47-56- |
| QAPGKGLEYVSGISPTGTSTYYANSVKGRFTISRDNPKNT | VH |
| LFLQMSSLRAEDTAVYYCVRVKGAYRGGMDVWGQGT | |
| TVTVSS(SEQ ID NO: 196) | |
| EVQLLESGGGLVQPGGSLRLSCAASGFTLSSYAMHWVR | STSA-C27-33-33- |
| QAPGKGLEYVSGISSSGSSTYYANSVKGRFTISRDNPKNT | VH |
| LFLQMSSLRAEDTAVYYCVRVKVAYRGGMDVWGQGT | |
| TVTVSS(SEQ ID NO: 197) | |
| EVQLLESGGGLVQPGGSLRLSCAASGFTLSSYAMHWVR | STSA-C27-56-56- |
| QAPGKGLEYVSGISPSSTSTYYANSVKGRFTISRDNPKNT | VH |
| LFLQMSSLRAEDTAVYYCVRVKVLYRGGMDVWGQGTT | |
| VTVSS(SEQ ID NO: 198) | |
| EVQLLESGGGLVQPGGSLRLSCAASGFTLSSYAMHWVR | STSA-C27-78-78- |
| QAPGKGLEYVSGISPSSASTYYANSVKGRFTISRDNPKNT | VH |
| LFLQMSSLRAEDTAVYYCVRVKSKYRGGMDVWGQGTT | |
| VTVSS(SEQ ID NO: 199) | |
| EVQLLESGGGLVQPGGSLRLSCAASGFTLSSYAMHWVR | STSA-C27-82-58- |
| QAPGKGLEYVSGISGNSASTYYANSVKGRFTISRDNPKN | VH |
| TLFLQMSSLRAEDTAVYYCVRVKLKYRGGMDVWGQGT | |
| TVTVSS(SEQ ID NO: 200) | |
| EVQLLESGGGLVQPGGSLRLSCAASGFTLSSYAMHWVR | STSA-C27-54-54- |
| QAPGKGLEYVSGISHSGTSTYYANSVKGRFTISRDNPKN | VH |
| TLFLQMSSLRAEDTAVYYCVRVRVLYRGGMDVWGQGT | |
| TVTVSS(SEQ ID NO: 201) | |
| EVQLLESGGGLVQPGGSLRLSCAASGFTLSSYAMHWVR | STSA-C27-36-36- |
| QAPGKGLEYVSGISPSGVSTYYANSVKGRFTISRDNPKN | VH |
| TLFLQMSSLRAEDTAVYYCVRVKVKYRGGMDVWGQG | |
| TTVTVSS(SEQ ID NO: 194) | |
| EVQLLESGGGLVQPGGSLRLSCAASGFTLSSYAMHWVR | STSA-C27-53-53- |
| QAPGKGLEYVSGISSNGGSTYYANSVKGRFTISRDNPKN | VH |
| TLFLQMSSLRAEDTAVYYCVRVFVRYRGGMDVWGQGT | |
| TVTVSS(SEQ ID NO: 202) | |
| EVQLLESGGGLVQPGGSLRLSCAASGFTLSSYAMHWVR | STSA-C27-67-67- |
| QAPGKGLEYVSGISPTSASTYYANSVKGRFTISRDNPKNT | VH |
| LFLQMSSLRAEDTAVYYCVRVKGRYRGGMDVWGQGTT | |
| VTVSS(SEQ ID NO: 203) | |
| EVQLLESGGGLVQPGGSLRLSCAASGFTLSSYAMHWVR | STSA-C27-55-55- |
| QAPGKGLEYVSGISPTGGSTYYANSVKGRFTISRDNPKN | VH |
| TLFLQMSSLRAEDTAVYYCVRVKGRYRGGMDVWGQGT | |
| TVTVSS(SEQ ID NO: 204) | |
| EVQLLESGGGLVQPGGSLRLSCAASGFTLSSYAMHWVR | STSA-C27-59-59- |
| QAPGKGLEYVSGISHSGNSTYYANSVKGRFTISRDNPKN | VH |
| TLFLQMSSLRAEDTAVYYCVRVKRRYRGGMDVWGQGT | |
| TVTVSS(SEQ ID NO: 205) | |
| EVQLLESGGGLVQPGGSLRLSCAASGFTLSSYAMHWVR | STSA-C27-58-58- |
| QAPGKGLEYVSGISPSSNSTYYANSVKGRFTISRDNPKNT | VH |
| LFLQMSSLRAEDTAVYYCVRVKVRYRGGMDVWGQGTT | |
| VTVSS(SEQ ID NO: 206) | |
| EVQLLESGGGLVQPGGSLRLSCAASGFTLSSYAMHWVR | STSA-C27-52-52- |
| QAPGKGLEYVSGISSSGSSTYYANSVKGRFTISRDNPKNT | VH |
| LFLQMSSLRAEDTAVYYCVRVKPAYRGGMDVWGQGTT | |
| VTVSS(SEQ ID NO: 207) | |
| EVQLLESGGGLVQPGGSLRLSCAASGFTLSSYAMHWVR | STSA-C27-Y2-Y2- |
| QAPGKGLEYVSGISYSSASTYYANSVKGRFTISRDNPKN | VH |
| TLFLQMSSLRAEDTAVYYCVRVKVRYRGGMDVWGQGT | |
| TVTVSS(SEQ ID NO: 208) | |
| ETTLTQSPDTLPLSPGDRASLSCRASQSVSSAYLAWYQQ | STSA-C27-VL |
| KPGQAPRLLIYGTSRRATGVPGRFSGSGSGTDFTLTISRL | |
| EPEDFAVYYCQLYGSSSVTFGQGTKLEIK(SEQ ID NO: | |
| 209) | |
| EIVLTQSPGTLSLSPGERATLSCRASQGISSAYLAWYQQK | STSA-C27-6-33-VL |
| PGQAPRLLIYGTSRRATGIPDRFSGSGSGTDFTLTISRLEP | |
| EDFAVYYCQLYGATSVTFGQGTKLEIK(SEQ ID NO: 210) | |
| EIVLTQSPGTLSLSPGERATLSCRASQGISSAYLAWYQQK | STSA-C27-7-33-VL |
| PGQAPRLLIYGTSRRATGIPDRFSGSGSGTDFTLTISRLEP | |
| EDFAVYYCQLYGATSVTFGQGTKLEIK(SEQ ID NO: 210) | |
| EIVLTQSPGTLSLSPGERATLSCRASQSVSSAYLAWYQQ | STSA-C27-24-56- |
| KPGQAPRLLIYGTSRRATGIPDRFSGSGSGTDFTLTISRLE | VL |
| PEDFAVYYCQLYGASSVTFGQGTKLEIK(SEQ ID NO: | |
| 211) | |
| EIVLTQSPGTLSLSPGERATLSCRASQSVSSAYLAWYQQ | STSA-C27-47-56- |
| KPGQAPRLLIYGTSRRATGIPDRFSGSGSGTDFTLTISRLE | VL |
| PEDFAVYYCQLYGASSVTFGQGTKLEIK(SEQ ID NO: | |
| 211) | |
| EIVLTQSPGTLSLSPGERATLSCRASQGISSAYLAWYQQK | STSA-C27-33-33- |
| PGQAPRLLIYGTSRRATGIPDRFSGSGSGTDFTLTISRLEP | VL |
| EDFAVYYCQLYGATSVTFGQGTKLEIK(SEQ ID NO: 210) | |
| EIVLTQSPGTLSLSPGERATLSCRASQSVSSAYLAWYQQ | STSA-C27-56-56- |
| KPGQAPRLLIYGTSRRATGIPDRFSGSGSGTDFTLTISRLE | VL |
| PEDFAVYYCQLYGASSVTFGQGTKLEIK(SEQ ID NO: | |
| 211) | |
| EIVLTQSPGTLSLSPGERATLSCRASQSISTAYLAWYQQK | STSA-C27-78-78- |
| PGQAPRLLIYGTSRRATGIPDRFSGSGSGTDFTLTISRLEP | VI |
| EDFAVYYCQLYGASSVTFGQGTKLEIK(SEQ ID NO: 212) | |
| EIVLTQSPGTLSLSPGERATLSCRASQDISSAYLAWYQQK | STSA-C27-82-58- |
| PGQAPRLLIYGTSRRATGIPDRFSGSGSGTDFTLTISRLEP | VL |
| EDFAVYYCQLYGATSVTFGQGTKLEIK(SEQ ID NO: 213) | |
| EIVLTQSPGTLSLSPGERATLSCRASQDVSSAYLAWYQQ | STSA-C27-54-54- |
| KPGQAPRLLIYGTSRRATGIPDRFSGSGSGTDFTLTISRLE | VL |
| PEDFAVYYCQLYGATSVTFGQGTKLEIK(SEQ ID NO: | |
| 214) | |
| EIVLTQSPGTLSLSPGERATLSCRASQNISTAYLAWYQQK | STSA-C27-36-36- |
| PGQAPRLLIYGTSRRATGIPDRFSGSGSGTDFTLTISRLEP | VL |
| EDFAVYYCQLYGATSVTFGQGTKLEIK(SEQ ID NO: 215) | |
| EIVLTQSPGTLSLSPGERATLSCRASQDASNAYLAWYQQ | STSA-C27-53-53- |
| KPGQAPRLLIYGTSRRATGIPDRFSGSGSGTDFTLTISRLE | VL |
| PEDFAVYYCQLYGSSSVTFGQGTKLEIK(SEQ ID NO: 216) | |
| EIVLTQSPGTLSLSPGERATLSCRASQGVSSAYLAWYQQ | STSA-C27-67-67- |
| KPGQAPRLLIYGTSRRATGIPDRFSGSGSGTDFTLTISRLE | VL |
| PEDFAVYYCQLYGRSSVTFGQGTKLEIK(SEQ ID NO: | |
| 217) | |
| EIVLTQSPGTLSLSPGERATLSCRASQNISTAYLAWYQQK | STSA-C27-55-55- |
| PGQAPRLLIYGTSRRATGIPDRFSGSGSGTDFTLTISRLEP | VL |
| EDFAVYYCQLYGTSSVTFGQGTKLEIK(SEQ ID NO: 218) | |
| EIVLTQSPGTLSLSPGERATLSCRASQSVSTAYLAWYQQ | STSA-C27-59-59- |
| KPGQAPRLLIYGTSRRATGIPDRFSGSGSGTDFTLTISRLE | VL |
| PEDFAVYYCQLYGATSVTFGQGTKLEIK(SEQ ID NO: | |
| 219) | |
| EIVLTQSPGTLSLSPGERATLSCRASQDISSAYLAWYQQK | STSA-C27-58-58- |
| PGQAPRLLIYGTSRRATGIPDRFSGSGSGTDFTLTISRLEP | VI |
| EDFAVYYCQLYGATSVTFGQGTKLEIK(SEQ ID NO: 213) | |
| EIVLTQSPGTLSLSPGERATLSCRASQGVSTAYLAWYQQ | STSA-C27-52-52- |
| KPGQAPRLLIYGTSRRATGIPDRFSGSGSGTDFTLTISRLE | VL |
| PEDFAVYYCQLYGATSVTFGQGTKLEIK(SEQ ID NO: | |
| 220) | |
| EIVLPQSPGTLSLSPGERATLSCRASQGVSSAYLAWYQQ | STSA-C27-Y2-Y2- |
| KPGQAPRLLIYGTSRRATGIPDRFSGSGSGTDFTLTISRLE | VL |
| PEDFAVYYCQL YGSTSVTFGQGTKLEIK(SEQ ID NO: | |
| 221) | |

In certain embodiments, an antibody or antigen-binding fragment thereof of the disclosure comprises heavy chain variable region (HCVR) and light chain variable region (LCVR) sequence pairs (HCVR/LCVR) selected from the group consisting of: Y0188-1/Y0188-1; Y0188-2/Y0188-2; Y0188-3/Y0188-3; Y0188-4/Y0188-4; Y0188-6/Y0188-6; Y0188-8/Y0188-8; Y0188-9/Y0188-9; Y0188-10/Y0188-10; Y0188-14/Y0188-14; HV3-15-14/Y01-14; HV3-15-14/164-14; HV3-15-14/KV4-14; HV3-15-14/KV1-27-14; HV3-15-14/KV1-9-14; HV3-15-14/KV1-NL1-14; HV3-15-14/KVID-43-14; HV3-48-14/Y01-14; HV3-48-14/164-14; HV3-48-14/KV4-14; HV3-48-14/KV1-27-14; HV3-48-14/KV1-9-14; HV3-48-14/KV1-NL1-14; HV3-48-14/KV1D-43-14; HV3-73*2-14/Y01-14; HV3-73*2-14/164-14; HV3-73*2-14/KV4-14; HV3-73*2-14/KV1-27-14; HV3-73*2-14/KV1-9-14; HV3-73*2-14/KV1-NL1-14; HV3-73*2-14/KVID-43-14; HV3-72-14/Y01-14; HV3-72-14/164-14; HV3-72-14/KV4-14; HV3-72-14/KV1-27-14; HV3-72-14/KV1-9-14; HV3-72-14/KV1-NL1-14; HV3-72-14/KV1D-43-14; Y01-14/Y01-14; Y01-14/164-14; Y01-14/KV4-14; Y01-14/KV1-27-14; Y01-14/KV1-9-14; Y01-14/KV1-NL1-14; Y01-14/KV1D-43-14; 162-14/Y01-14; 162-14/164-14; 162-14/KV4-14; 162-14/KV1-27-14; 162-14/KV1-9-14; 162-14/KV1-NL1-14; 162-14/KVID-43-1L; VH73-14/Y01-14; VH73-14/164-14; VH73-14/KV4-14; VH73-14/KV1-27-14; VH73-14/KV1-9-14; VH73-14/KV1-NL1-14; and VH73-14/KVID-43-14.

The antibodies recited below in Table 6 are described in more detail in WO2021/213329, incorporated herein by reference in its entirety for all purposes.

TABLE 6

| | |
|---|---|
| Y0188-1 | EVQLVESGGGLVQPKGSLKLSCAASGFTFNTYGMHWVRQAPG |
| VH | KGLEWVAHIRSKSSNYATYYADSVKDRFTISRDDSQSMLYLQM |
| | NNLKTEDTAMYYCVRWFRAMDYWGQGTSVTVSS(SEQ ID NO: |
| | 222) |
| Y0188-2 | EVQLIESGGGLVQPKGSLKLSCAASGFTFNMYAMDWVRQAPGK |
| VH | GLEWVARIRSKGSNFETNYADSVKDRFTISRDDSQSMVYLQMIN |
| | LKTEDTAMYYCVRHRGGAWFAYWGQGTLVSVSA(SEQ ID NO: |
| | 223) |
| Y0188-3 | QVQLVETGGGLVRPGNSLKLSCVTSGFTFSNYRMHWLRQPPGK |
| VH | RLEWIAVITVKSNNYGANYAESVKGRFAISRDDSKSSVYLEMNR |
| | LREEDTATYFCSRERAYGNPFDYWGQGTTLTVSS(SEQ ID NO: |
| | 224) |
| Y0188-4 | EVQLVESGGGLVQPKGSLKLSCAASGFTFNMYAMNWVRQAPG |
| VH | QGLEWVARIRSKSNNYATYYADSVKDRFIISRDDSESMVYLQMS |
| | NLRAADTAMYYCVRHLRAMDYWGQGTSVTVSS(SEQ ID NO: |
| | 225) |
| Y0188-6 | EVQLVESGGGLVQPKGSLKLSCAASGFSFNMYAMNWVRQAPG |
| VH | KGLEWVARIRTKSNHYSTYYADSVKDRFTISRDDSASMFYLQM |
| | NNLKTEDTAMYFCVRHLRAMDYWGQGTSVTVSS(SEQ ID NO: |
| | 226) |
| Y0188-8 | EVQLIESGGGLVQPKGSLKLSCAASGFTFNMYAMDWVRQAPGK |
| VH | GLEWVARIRSKGSNFETNYADSVKDRFTISRDDSQSMVYLQMN |
| | NLKTEDTAMYYCVRHRGGAWFAYWGQGTLVTVSA(SEQ ID |
| | NO: 227) |
| Y0188-9 | EVQLVESGGGLVRPKGSLKLSCAASGFSFNTYAMNWVRQAPGK |
| VH | GLEWIVWIRSKSHNYATYYADSVKDRFTISRDDSESMLYLQMN |
| | NLKTEDTAMYYCVRHLRAMDYWGQGTSVTVSS(SEQ ID NO: |
| | 228) |
| Y0188-10 | EVRLVESGGGLVQPKGSLKLSCEASGFSFNMYAMNWVRQAPG |
| VH | KGLEWITHIRSKSNNYATYYADSVKDRFIISRDDSESMVYLQMN |
| | NLKTEDTAMYYCVRLLRALDYWGQGTSVTVSS(SEQ ID NO: |
| | 229) |
| Y0188-14 | EVQLVESGGGLVQPKGSLKLSCAASGFTFNMYGMHWVRQAPG |
| VH | KGLEWVAHIRSKSSNYATYYADSVKDRLTISRDDSQSMLYLQM |
| | NNLKTEDTAMYYCVRWFRAMDYWGQGTSVTVSS(SEQ ID NO: |
| | 230) |
| HV3-15-14 | EVQLVESGGGLVKPGGSLRLSCAASGFTFSMYGMHWVRQAPG |
| VH | KGLEWVGHIRSKSSNYATYYADSVKDRFTISRDDSKNTLYLQM |
| | NSLKTEDTAVYYCTTWFRAMDYWGQGTLVTVSS(SEQ ID NO: |
| | 231) |
| HV3-48-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS(SEQ ID NO: |
| | 232) |
| HV3-73*2- | EVQLVESGGGLVQPGGSLKLSCAASGFTFSMYGMHWVRQASG |
| 14 | KGLEWVGHIRSKSSNYATYYADSVKDRFTISRDDSKNTAYLQM |
| VH | NSLKTEDTAVYYCTRWFRAMDYWGQGTLVTVSS(SEQ ID NO: |
| | 233) |
| HV3-72-14 | EVQLVESGGGLVQPGGSLRLSCAASGFTFSMYGMHWVRQAPG |
| VH | KGLEWVGHIRSKSSNYATYYADSVKDRFTISRDDSKNSLYLQM |
| | NSLKTEDTAVYYCARWFRAMDYWGQGTLVTVSS(SEQ ID NO: |
| | 234) |
| Y01-14 | EVQLVESGGGLVQPGGSLRLSCAASGFTFSMYGMHWVRQAPG |
| VH | KGLEWVSHIRSKSSNYATYYADSVKDRFTISRDNAKNSLYLQM |
| | NSLRAEDTAVYYCARWFRAMDYWGQGTLVTVSS(SEQ ID NO: |
| | 232) |
| 162-14 | EVQLVESGGGLEQPGGSLRLSCAGSGFTFRMYGMHWVRQAPG |
| VH | KGLEWVSHIRSKSSNYATYYADSVKDRFTISRDNSKNTLYLQM |
| | NSLRAEDTAVYYCAKWFRAMDYWGQGTTVTVSS(SEQ ID NO: |
| | 235) |
| VH73-14 | EVQLVESGGGLVQPGGSLKLSCAASGFTFSMYGMHWVRQASG |
| VH | KGLEWVGHIRSKSSNYATYYADSVKDRFTISRDDSKNTAYLQM |
| | NSLKTEDTAVYYCTRWFRAMDYWGQGTTVTVSS(SEQ ID NO: |
| | 236) |
| Y0188-1 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK(SEQ ID NO: 237) |
| Y0188-2 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK(SEQ ID NO: 238) |
| Y0188-3 | DIQMTQSPSSLSASLGERVSLTCRASQEISGYLSWLQQKPDGTIK |
| VL | RLIYAASTLDSGVPKRFSGSRSGSDYSLTISSLESEDFADYYCLQY |
| | GSYPYTFGGGTKLEIK(SEQ ID NO: 239) |
| Y0188-4 | DIVLTQSPASLTVSLGQRATISCRASKSVSTSGYSYMHWYQQKP |
| VL | GQPPKLLIYLASNLESGVPARFSGSGSGTDFTLNIHPVEEEDAAT |
| | YYCQHSRELPITFGSGTKLEIK(SEQ ID NO: 240) |
| Y0188-6 | DIVLTQSPASLVVSLGQRATISCRASQSVSTSGYSYMHWYQQKP |
| VL | GQPPKLLIYLASNVQSGVPARFSGSGSGTDFTLNIHPVEEEDVAT |
| | YYCHHNRDLPFTFGSGTKLEIK(SEQ ID NO: 241) |
| Y0188-8 | DIVVTQSPASLAVSLGQRATISCRASKSVSTSGYSYMHWYQQKP |
| VL | GQPPKLLIYLASNLESGVPARFSGSGSGTDFTLNIHPVEEEDVAIY |
| | YCQHSRELPLTFGAGTKLELK(SEQ ID NO: 238) |
| Y0188-9 | DIVLTQSPASLAVSLGQRATISCRASKSVSASGYSYMHWYQQKP |
| VL | GQPPKLLIYLASNLQSGVPARFSGSGSGTDFTLNIHPVEEEDAAT |
| | YYCQHSRELPPTFGGGTKLEIK(SEQ ID NO: 242) |
| Y0188-10 | DIVLTQSPASLAVFLGQRATISCRASKSVSTSGYSYMHWYQQKA |
| VL | GQPPKLLIYLASNLESGVPARFSGSGSGTDFTLNIHPVEEEDAAT |
| | YYCHHSRELPITFGSGTKLEMK(SEQ ID NO: 243) |
| Y0188-14 | DIVMTQSHKFMSTSVGDRVSITCKASQDVSTAVAWYQEKPGQS |
| VL | PKLLIYWASTRHTGVPDRFTGSGSGTDYTLTISSVQAEDLALYY |
| | CQQHYSTPLTFGAGTKLELK(SEQ ID NO: 237) |
| Y01-14 | EIVLTQSPGTLSLSPGERATLSCKASQDVSTAVAWYQQKPGQAP |
| VL | RLLIYWASTRHTGIPDRFSGSGSGTDFTLTISRLEPEDFAVYYCQ |
| | QHYSTPLTFGQGTKVEIK(SEQ ID NO: 244) |
| 164-14 | DIVMTQSPLSLPVTPGEPASISCKASQDVSTAVAWYLQKSGQSP |
| VL | QLLIYWASTRHTGVPDRFSGSGSGTDFTLKISRVEAEDVGFYYC |
| | QQHYSTPLTFGQGTKLEIK(SEQ ID NO: 245) |
| KV4-14 | DIVMTQSPDSLAVSLGERATINCKASQDVSTAVAWYQQKPGQP |
| VL | PKLLIYWASTRHTGVPDRFSGSGSGTDFTLTISSLQAEDVAVYYC |
| | QQHYSTPLTFGGGTKVEIK(SEQ ID NO: 246) |
| KV1-27-14 | DIQMTQSPSSLSASVGDRVTITCKASQDVSTAVAWYQQKPGKVP |
| VL | KLLIYWASTRHTGVPSRFSGSGSGTDFTLTISSLQPEDVATYYCQ |
| | QHYSTPLTFGGGTKVEIK(SEQ ID NO: 247) |
| KV1-9-14 | DIQLTQSPSFLSASVGDRVTITCKASQDVSTAVAWYQQKPGKAP |
| VL | KLLIYWASTRHTGVPSRFSGSGSGTEFTLTISSLQPEDFATYYCQ |
| | QHYSTPLTFGGGTKVEIK(SEQ ID NO: 248) |
| KV1-NL1- | DIQMTQSPSSLSASVGDRVTITCKASQDVSTAVAWYQQKPGKAP |
| 14 | KLLLYWASTRHTGVPSRFSGSGSGTDYTLTISSLQPEDFATYYCQ |
| VL | QHYSTPLTFGGGTKVEIK(SEQ ID NO: 249) |
| KV1D-43- | AIRMTQSPFSLSASVGDRVTITCKASQDVSTAVAWYQQKPAKAP |
| 14 | KLFIYWASTRHTGVPSRFSGSGSGTDYTLTISSLQPEDFATYYCQ |
| VL | QHYSTPLTFGGGTKVEIK(SEQ ID NO: 250) |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat CSU in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. Pat. No. 8,945,559.

### Dosage

The amount of IL-4R antagonist (e.g., anti-IL-4R antibody) administered to a subject according to the methods described herein is, generally, a therapeutically effective amount. As used herein, the phrase “therapeutically effective amount” means an amount of IL-4R antagonist that results in improvement in one or more ColdU-associated PRO measures (as defined elsewhere herein). A “therapeutically effective amount” also includes an amount of IL-4R antagonist that inhibits, prevents, lessens, or delays the progression of ColdU in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, 200 to 300 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, two or more (e.g., 2, 3, 4, or 5 or more) doses are administered at the beginning of the treatment regimen as “initial doses” or “loading doses” followed by subsequent doses that are administered on a less frequent basis (e.g., “maintenance doses”). In one embodiment, the maintenance dose may be lower than the loading or initial dose. For example, one or more loading doses of 600 mg of IL-4R antagonist may be administered followed by maintenance doses of about 75 mg to about 300 mg. In certain embodiments, the methods comprise an initial dose or loading dose of about 400 mg or about 600 mg of an IL-4R antagonist. In certain embodiments, the methods comprise one or more secondary doses or maintenance doses of about 200 mg or about 300 mg of the IL-4R antagonist.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of more than 30 kg, and the IL-4R antagonist is administered at an initial dose of about 400 mg and one or more secondary doses of about 400 mg, and the secondary doses are administered every other week (q2w). In certain exemplary embodiments, a subject is a pediatric subject having a body weight of more than 30 kg, and the IL-4R antagonist is administered at an initial dose of about 300 mg and one or more secondary doses of about 300 mg, and the secondary doses are administered every other week (q2w). In certain exemplary embodiments, a subject is a pediatric subject having a body weight of more than 30 kg, and the IL-4R antagonist is administered at an initial dose of about 200 mg and one or more secondary doses of about 400 mg, and the secondary doses are administered every other week (q2w). In particularly exemplary embodiments, a subject is a pediatric subject having a body weight of more than 30 kg, and the IL-4R antagonist is administered at an initial dose or loading dose of about 400 mg and one or more secondary doses or maintenance doses of about 200 mg, and the secondary doses are administered every other week (q2w).

In certain exemplary embodiments, a subject is a pediatric subject having a body weight of 30 kg or less and a body weight of at least 15 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of 30 kg or less and a body weight of at least 15 kg, and the IL-4R antagonist is administered at an initial dose of about 600 mg and one or more secondary doses of about 600 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 500 mg and one or more secondary doses of about 500 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 400 mg and one or more secondary doses of about 400 mg, and the secondary doses are administered every four weeks (q4w). In certain exemplary embodiments, a subject is a pediatric subject having a body weight of 30 kg or less and a body weight of at least 15 kg, and the IL-4R antagonist is administered at an initial dose of about 300 mg and one or more secondary doses of about 300 mg, and the secondary doses are administered every four weeks (q4w). In particularly exemplary embodiments, a subject is a pediatric subject having a body weight of 30 kg or less and a body weight of at least 15 kg, and the IL-4R antagonist is administered at an initial dose of about 600 mg and one or more secondary doses or maintenance doses of about 300 mg, and the secondary doses are administered every four weeks (q4w).

In certain exemplary embodiments, a subject is a pediatric subject having a body weight of less than 15 kg and at least 5 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In certain exemplary embodiments, a subject is a pediatric subject having a body weight of less than 15 kg and at least 5 kg, and the IL-4R antagonist is administered at an initial dose of about 300 mg and one or more secondary doses of about 300 mg, and the secondary doses are administered every four weeks (q4w). In certain exemplary embodiments, a subject is a pediatric subject having a body weight of less than 15 kg and at least 5 kg, and the IL-4R antagonist is administered at an initial dose of about 250 mg and one or more secondary doses of about 250 mg, and the secondary doses are administered every four weeks (q4w). In particularly exemplary embodiments, a subject is a pediatric subject having a body weight of less than 15 kg and at least 5 kg, and the IL-4R antagonist is administered at an initial dose of about 200 mg and one or more secondary doses of about 200 mg, and the secondary doses are administered every four weeks (q4w).

In certain exemplary embodiments, a subject is an adolescent subject having a body weight of less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some exemplary embodiments, a subject is an adolescent subject having a body weight of less than 60 kg, and the IL-4R antagonist is administered at an initial dose of about 400 mg and one or more secondary doses or maintenance doses of about 200 mg, and the secondary doses are administered every other week (q2w). In certain exemplary embodiments, a subject is an adolescent subject having a body weight that is greater than or equal to 30 kg and less than 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In some exemplary embodiments, a subject is an adolescent subject having a body weight that is greater than or equal to 30 kg and less than 60 kg, and the IL-4R antagonist is administered at an initial dose of about 400 mg and one or more secondary doses or maintenance doses of about 200 mg, and the secondary doses are administered every other week (q2w).

In certain exemplary embodiments, a subject is an adolescent subject having a body weight of at least 60 kg, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In particularly exemplary embodiments, a subject is an adolescent subject having a body weight of at least 60 kg, and the IL-4R antagonist is administered at an initial dose of about 600 mg and one or more secondary doses or maintenance doses of about 300 mg, and the secondary doses are administered every other week (q2w).

In certain exemplary embodiments, a subject is an adult, and the IL-4R antagonist is administered at a dose of about 50 mg, about 100 mg, about 150 mg, about 200 mg, about 250 mg, about 300 mg, about 350 mg, about 400 mg, about 450 mg, about 500 mg, about 550 mg, or about 600 mg. In particularly exemplary embodiments, a subject is an adult, and the IL-4R antagonist is administered at an initial dose of about 600 mg and one or more secondary doses or maintenance doses of about 300 mg, and the secondary doses are administered every other week (q2w).

In certain exemplary embodiments, an IL-4R antagonist is administered at a concentration of 150 mg/mL using a prefilled device. In some embodiments, a 150 mg/ml IL-4R antagonist solution in a pre-filled device is used to deliver 300 mg IL-4R antagonist in a 2 mL injection. In certain exemplary embodiments, an IL-4R antagonist is administered at a concentration of 175 mg/mL using a prefilled device. In some embodiments, a 175 mg/mL IL-4R antagonist solution in a pre-filled device is used to deliver 200 mg IL-4R antagonist in a 1.14 mL injection.

### Combination Therapies

Certain embodiments of the methods described herein comprise administering to the subject one or more additional therapeutic agents in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat ColdU or an associated condition or complication comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

In exemplary embodiments, an additional therapeutic agent administered in combination with the IL-4R antagonist is a background therapy. In exemplary embodiments, a background therapy includes one or both of an antihistamine and an anti-IgE antibody. In certain embodiments, the method leads to reduced need of the background therapy. For example, in certain embodiments, the method leads to reduced dose and/or reduced frequency of the background therapy.

The additional therapeutic agent may be, e.g., another IL-4R antagonist (e.g., one or more suitable IL-4R antagonists listed in Tables 1-4), an IgE antagonist, an antihistamine, an IL-1 antagonist (including, e.g., an IL-1 antagonist as set forth in U.S. Pat. No. 6,927,044), an IL-5 antagonist, an IL-5R antagonist, an IL-6 antagonist, an IL-6R antagonist (including, e.g., an anti-IL-6R antibody as set forth in U.S. Pat. No. 7,582,298), or an IL-17 antagonist.

In an exemplary embodiment, the additional therapeutic is an H1 antihistamine. In some embodiments, the H1 antihistamine is selected from the group consisting of cetirizine, levocetirizine, fexofenadine, loratadine, desloratadine, bilastine, and rupatadine.

In a further exemplary embodiment, the additional therapeutic is an anti-IgE antibody. In some embodiments, the anti-IgE antibody is omalizumab. In some embodiments, the anti-IgE antibody is ligelizumab.

In some embodiments, an additional therapeutic agent administered in combination with the IL-4R antagonist is a vaccine. In certain exemplary embodiments, the vaccine is a viral vaccine or a bacterial vaccine. In certain exemplary embodiments, the vaccine is a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHIB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PPSV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RVI (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html). In some embodiments, the vaccine is for tetanus, diphtheria, pertussis and/or seasonal trivalent/quadrivalent influenza vaccine.

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine is concurrently treated for CSU with an IL-4R antagonist.

In certain embodiments, treatment with an IL-4R antagonist is suspended or terminated prior to treatment with the vaccine. In certain embodiments, treatment with the IL-4R antagonist is suspended about 1 to about 9 (e.g., about 1, about 11/2, about 2, about 21/2, about 3, about 31/2, about 4, about 41/2, about 5, about 51/2, about 6, about 61/2, about 7, about 71/2, about 8, about 81/2, about 9, or more) weeks prior to administration of the vaccine. In some embodiments, treatment with the IL-4R antagonist is suspended about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, treatment with the IL-4R antagonist is resumed subsequent to treatment with the vaccine. In certain embodiments, treatment with the IL-4R antagonist is resumed about 1 to about 14 (e.g., about 1, about 11/2, about 2, about 21/2, about 3, about 31/2, about 4, about 41/2, about 5, about 51/2, about 6, about 61/2, about 7, about 71/2, about 8, about 81/2, about 9, about 91/2, about 10, about 101/2, about 11, about 111/2, about 12, about 121/2, about 13, about 131/2, about 14, about 141/2, or more) weeks subsequent to administration of the vaccine. In some embodiments, treatment with the IL-4R antagonist is resumed about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

In certain embodiments, the effectiveness of the IL-4R antagonist is not decreased by administration in combination with the vaccine, or by subsequent administration of the vaccine.

In some embodiments, the effectiveness of the vaccine is not decreased by administration in combination with the IL-4R antagonist, or by previous and/or subsequent administration of the IL-4R antagonist. In some embodiments, the subject develops seroprotective neutralization titers to the vaccine when the vaccine is co-administered with the IL-4R antagonist.

In certain exemplary embodiments, a subject is administered a vaccine described herein, wherein before, during, or after administration of the vaccine, the subject is administered at least one dose of IL-4R antagonist.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (q10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n×7 days)+3 days, e.g., (n×7 days)+2 days, (n×7 days)+1 day, or (n×7 days), wherein “n” designates the number of weeks, e.g., 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist, or may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 600 mg or 400 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 300 mg or 200 mg of IL-4R antagonist may be administered followed by secondary doses/maintenance doses of about 300 mg or about 200 mg, respectively. In one embodiment, a loading dose may be split, e.g., two or more doses administered at different time points, e.g., two loading doses wherein a second loading dose is administered two weeks after a first loading dose.

In certain embodiments, the initial dose is about 50 mg to about 600 mg of the IL-4R antagonist. In one embodiment, the initial dose is 600 mg of the IL-4R antagonist. In another embodiment, the initial dose is 400 mg of the IL-4R antagonist.

In certain embodiments, the secondary dose(s) are about 50 mg to about 600 mg of the IL-4R antagonist. In one embodiment, the maintenance dose is 300 mg of the IL-4R antagonist. In one embodiment, the maintenance dose is 200 mg of the IL-4R antagonist.

In certain embodiments, an initial dose is three times a maintenance dose. In certain embodiments, an initial dose is two times a maintenance dose. In certain embodiments, an initial dose is equal to a maintenance dose.

In some embodiments, the subject is a child and has a body weight of less than 15 kg and at least 5 kg, the initial dose comprises 200 mg of the antibody or antigen-binding fragment thereof, and the one or more secondary doses comprises 200 mg of the antibody or antigen-binding fragment thereof administered every four weeks (q4w).

In some embodiments, the subject is a child and has a body weight of 30 kg or less and at least 15 kg, the initial dose comprises 600 mg of the antibody or antigen-binding fragment thereof, and the one or more secondary doses comprises 300 mg of the antibody or antigen-binding fragment thereof administered every four weeks (q4w).

In some embodiments, the subject is a child and has a body weight of 30 kg or less and at least 15 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject is a child and has a body weight of greater than 30 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w). In exemplary embodiments, the subject is an adolescent and has a body weight that is greater than or equal to 30 kg and less than 60 kg, the initial dose comprises 400 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject is an adolescent and has a body weight of more than 60 kg, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In some embodiments, the subject is an adult, the initial dose comprises 600 mg of the antibody or antigen-binding fragment thereof, and the one or more secondary doses comprises 300 mg of the antibody or antigen-binding fragment thereof administered every other week (every other week is used interchangeably with every two weeks, bi-weekly or q2w).

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-4R antagonist and a second therapeutic agent, to a patient to treat ColdU (e.g., CIndU) or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 600 mg of the IL-4R antagonist may be administered after which one or more doses (e.g., 2, 3, 4, 5, 6, 7, 8, or more) of a second therapeutic agent (e.g., an H1 antihistamine or an anti-IgE antibody, as described elsewhere herein) may be administered to treat, alleviate, reduce or ameliorate one or more symptoms of ColdU. In some embodiments, the IL-4R antagonist is administered at one or more doses (e.g., 2, 3, 4, 5, 6, 7, 8, or more) resulting in an improvement in one or more ColdU-associated parameters followed by the administration of a second therapeutic agent to prevent recurrence of at least one symptom of ColdU. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, the IL-4R antagonist is administered for at least 24 weeks.

In certain embodiments, a kit comprising a dosage form of an antibody, or an antigen-binding fragment thereof, that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, for the treatment of ColdU is provided. In certain embodiments, the antibody or antigen-binding fragment thereof comprises a heavy chain variable region (HCVR) sequence of SEQ ID NO: 1 and a light chain variable region (LCVR) sequence of SEQ ID NO: 2. In certain embodiments, the antibody is dupilumab.

The kit can comprise a label or package insert, wherein the label or package insert comprises instructions for administering the dosage form for the treatment of ColdU. The instructions can recite a dosing regimen described further herein for the treatment of ColdU.

### Treatment Populations

The methods provided herein include administering to a subject in need thereof a therapeutic composition comprising an IL-4R antagonist. The expression “a subject in need thereof” means a human or non-human animal that exhibits one or more symptoms or indicia of ColdU, or who has been diagnosed with ColdU.

In certain exemplary embodiments, a subject in need thereof has a diagnosis of primary acquired ColdU defined as recurrence of itchy wheals and/or angioedema due to cold for longer than 6 weeks.

In certain exemplary embodiments, a subject in need thereof has a positive ice cube provocation test, i.e., presenting at least a confluent hive/wheal on the exposed skin area.

In a related embodiment, a “subject in need thereof” may be a subject who, prior to receiving an IL-4R antagonist, has been prescribed or is currently taking an antihistamine. In certain embodiments, the subject is currently taking an H1 antihistamine.

In certain exemplary embodiments, a subject in need thereof meets at least one of the following criteria despite use of an H1 antihistamine: Urticaria Control Test (UCT) (4-item) <12; documented medical history of cold exposure triggered anaphylaxis or oropharyngeal edema; documented medical history of cold exposure triggered urticaria requiring emergency medical care visit or treatment with epinephrine.

Suitable H1 antihistamines include, but are not limited to, fexofenadine, cetirizine, terfenadine, bilastine, chlorpheniramine, diphenhydramine, carbinoxamine, promathazine, desloratadine, dexchlorpheniramine, hydroxyzine, loratadine, levocetirizine, clemastine, ebastine, dexbrompheniramine, triprolidine, brompheniramine, trimeprazine, cyproheptadine, azelastine, cyproheptadine, emedastine, levocabastine, cinnarizine, rupatadine and the like. In a particular embodiment, a suitable antihistamine is selected from cinnarizine, rupatadine, bilastine, desloratadine and ebastine. In another particular embodiment, a suitable antihistamine is selected from rupatadine, bilastine, desloratadine and ebastine. In yet another embodiment, a suitable antihistamine is selected from loratadine, cetirizine and desloratadine. For example, methods that comprise administering an IL-4R antagonist to a patient who has been taking a regular course an H1 antihistamine for six or more weeks immediately preceding the administration of the IL-4R antagonist (such prior treatments are referred to herein as “′background treatments”) are provided.

In yet other embodiments, the amount of the H1 antihistamine is gradually decreased prior to or after the start of IL-4R antagonist administration.

In another exemplary embodiment, a “subject in need thereof” has a diagnosis of ColdU refractory to H1 antihistamines prior to receiving the IL-4R antagonist. In some embodiments, the ColdU symptoms of the subject persist despite treatment with an H1 antihistamine (i.e., the subject is resistant to treatment with an H1 antihistamine.)

In some embodiments, a “subject in need thereof” is selected from the group consisting of: a subject age 18 years old or older, a subject 12 years or older, a subject age 12 to 17 years old (12 to <18 years old), a subject age 6 to 11 years old (6 to <12 years old), a subject aged 2 to 11 years old (2 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months. In particularly exemplary embodiments, a subject is a child 6 to <12 years old (also referred to herein as a “pediatric” subject). In certain embodiments, a subject in need thereof is a child 6 to <12 years old having a body weight of more than 30 kg. In certain embodiments, a subject in need thereof is a child 6 to <12 years old having a body weight of 30 kg or less and at least 15 kg. In certain embodiments, a subject in need thereof is an adolescent 12 to <18 years old having a body weight of at least 60 kg. In exemplary embodiments, a subject in need thereof is an adolescent 12 to <18 years old having a body weight of less than 60 kg. In other exemplary embodiments, a subject in need thereof is an adolescent 12 to <18 years old having a body weight that is greater or equal to 30 kg and less than 60 kg.

In some embodiments, a “subject in need thereof” is a subject who is treated with a vaccine, e.g., viral vaccine or a bacterial vaccine. In some embodiments, the vaccine is a live vaccine, e.g., a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHIB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and cclIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PPSV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RVI (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html).

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine concurrently is treated for CSU with an IL-4R antagonist. In some embodiments, the subject suspends treatment with an IL-4R antagonist prior to administration of the vaccine.

In certain embodiments the subject suspends treatment with the IL-4R antagonist about 1 to about 9 (e.g., about 1, about 11/2, about 2, about 21/2, about 3, about 31/2, about 4, about 41/2, about 5, about 51/2, about 6, about 61/2, about 7, about 71/2, about 8, about 81/2, about 9, or more) weeks prior to administration of the vaccine. In certain embodiments, the subject suspends treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, the subject resumes treatment with the IL-4R antagonist subsequent to treatment with the vaccine. In certain embodiments, the subject resumes treatment with the IL-4R antagonist 1 to 14 (e.g., about 1, about 11/2, about 2, about 21/2, about 3, about 31/2, about 4, about 41/2, about 5, about 51/2, about 6, about 61/2, about 7, about 71/2, about 8, about 81/2, about 9, about 91/2, about 10, about 101/2, about 11, about 111/2, about 12, about 121/2, about 13, about 131/2, about 14, about 141/2, or more) weeks subsequent to administration of the vaccine. In certain embodiments, the subject resumes treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

### Methods for Assessing Pharmacodynamic ColdU-Associated Parameters

Methods for assessing one or more pharmacodynamic ColdU-associated parameters in a subject in need thereof, caused by administration of a pharmaceutical composition comprising an IL-4R antagonist, are provided. A reduction in the incidence of ColdU symptoms or an improvement in a ColdU-associated PRO measure may correlate with an improvement in one or more pharmacodynamic ColdU-associated parameters; however, such a correlation is not necessarily observed in all cases.

Examples of “pharmacodynamic ColdU-associated parameters” include, for example, the following: (a) biomarker expression levels and (b) serum protein and RNA analysis. An “improvement in a pharmacodynamic ColdU-associated parameter” means, for example, a decrease from baseline of one or more biomarkers, such as IgE, eosinophil level, c-reactive protein (CRP), IL-6, D-dimer, medium platelet volume (MPV), IL-17, IL-18, IL-31, IL-33, and metalloproteinase-9. As used herein, the term “baseline,” with regard to a pharmacodynamic ColdU-associated parameter, means the numerical value of the pharmacodynamic ColdU-associated parameter for a patient prior to or at the time of administration of a pharmaceutical composition described herein.

To assess a pharmacodynamic ColdU-associated parameter, the parameter is quantified at baseline and at a time point after administration of the pharmaceutical composition. For example, a pharmacodynamic ColdU-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic ColdU-associated parameter (e.g., an increase or decrease, as the case may be, depending on the specific parameter being measured).

In certain embodiments, administration of an IL-4R antagonist to a patient causes a change, such as a decrease or increase, in expression of a particular biomarker. ColdU-associated biomarkers include, but are not limited to total IgE, c-reactive protein (CRP), IL-6, D-dimer, medium platelet volume (MPV), IL-17, IL-18, IL-31, IL-33, and metalloproteinase-9. For example, administration of an IL-4R antagonist to a ColdU patient can cause a decrease in total serum IgE levels. The decrease can be detected at about week 1, about week 2, about week 3, about week 4, about week 5, or longer following administration of the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist or IL-4/IL-13 signaling (e.g., by measuring soluble IL-4Rα, IL-4, IL-13, etc.). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in U.S. Pat. No. 8,945,559, which is incorporated herein by reference in its entirety for all purposes.

The present disclosure is further illustrated by the following example which should not be construed as further limiting. The contents of the figures, tables and all references, patents and published patent applications cited throughout this application are expressly incorporated herein by reference for all purposes.

Furthermore, in accordance with the present disclosure there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, New York; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use the methods and compositions featured in the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Examples is the human anti-IL-4R antibody named dupilumab (also referred to herein as “mAb1” or DUPIXENT®).

### Example 1. a Randomized, Double-Blind, Placebo-Controlled, Multicenter, Parallel Group Study of Dupilumab in Patients with Chronic Inducible Cold Urticaria Who Remain Symptomatic Despite the Use of H1-Antihistamine Treatment

### Study Rationale

Chronic inducible urticaria (ColdU) is a subtype of chronic urticaria characterized by recurrent itchy wheals, angioedema, or both with a minimum disease duration of 6 weeks, that only occurs after exposure to defined external triggers and is classified according to the stimulus that provokes the development of signs and symptoms. Unlike chronic spontaneous urticaria (CSU), symptoms in ColdU patients (itchy wheals and angioedema) develop only and reproducibly in response to the trigger stimulus that is specific for their condition (e.g., cold exposure in cold urticaria (ColdU)). The triggers that lead to the urticarial signs and symptoms in ColdU patients are mainly physical or chemical stimuli, which results in 2 main sub-groups of ColdU: physical and non-physical, respectively. Physical triggers include pressure (in delayed pressure urticaria (DPU)), radiation (in solar urticaria), friction (in symptomatic dermographism), temperature (in cold and heat urticaria), and vibration (in vibratory angioedema). Chemical triggers of ColdU reactions include water (in aquagenic urticaria), sweat (in cholinergic urticaria (CholU)), and other urticariogenic chemical compounds (in contact urticaria).

ColdU has an estimated prevalence of about 0.5% in the general population, and has substantial impact on quality of life (QoL) in many patients, mainly due to the need for trigger avoidance. Within the group of ColdU, most ColdU subtypes are rare and therefore very difficult to study. The most common are symptomatic dermographism, cholinergic urticaria and ColdU (also called chronic inducible cold urticaria), ColdU being the second most common form of physical urticaria, with an estimated annual incidence of 0.05%.

ColdU manifests as wheals, angioedema, or both secondary to the release of mast cell mediators after exposure to cold or cooling and rewarming of the skin. The clinical symptoms occur within minutes after skin contact with cold air, cold liquids, cold solid objects, or evaporation-based cooling and persist in general for a few hours. In severe cases, patients can develop systemic involvement including anaphylaxis. Local angioedema affecting the lips, tongue, or pharyngeal tract has been associated with the ingestion of cold beverages or food, and shock like reactions are reported after swimming in cold water. Cold urticarias can be classified as those with typical responses to cold provocation tests, those with atypical responses to cold provocation, and those with familial forms.

Primary acquired ColdU is the most common form of ColdU and is considered idiopathic in nature. Historically the majority (96%) of patients with acquired cold-induced urticaria have been shown to have primary cold-induced urticaria, with only rare occurrences of secondary cold-induced urticaria. Secondary acquired ColdU is very rare and associated with an underlying disease most commonly cryoglobulinemia, but infectious causes, leukocytoclastic vasculitis, and some drug-induced cases have been reported. Both primary and secondary acquired ColdU have typical positive responses to cold provocation. Several other rare forms of atypical acquired ColdU exist, which have negative immediate tests to cold provocation and include systemic atypical acquired ColdU, cold-dependent dermographism, cold-induced ColdU, delayed ColdU, and localized cold-reflex urticaria. Lastly, hereditary forms of ColdU have been described: 1) Familial cold autoinflammatory syndrome caused by mutations in NLRP3 (cryopyrin) and is inherited in an autosomal dominant manner, which includes cryopyrin-associated periodic syndromes, Muckle-Wells syndrome and neonatal-onset multisystem inflammatory disease; and 2) Familial atypical cold urticaria inherited in an autosomal dominant fashion with having phospholipase C-g2-associated deficiency and immune dysregulation (PLAID).

Chronic inducible urticarias are diagnosed based on the patient history and the results of provocation testing. The treatment of inducible urticaria is trigger avoidance and prophylaxis by H1-antihistamines that prevent the effects of the mast cell-mediator histamine or, if antihistamines are not effective, agents that prevent the activation of mast cells (e.g., anti-immunoglobulin E (IgE) omalizumab). The consensus recommendations for ColdU management (EAACI/GA²LEN/EDF/UNEV) are mainly based on treatments approved for CSU, and some clinical data available in patients suffering from various types of ColdUs (cholinergic, symptomatic dermographism, ColdU). The recommended therapies are, in general, not approved for ColdU by regulatory agencies, with only limited specific countries where antihistamines are approved under broader “urticaria” indications. All patients are advised to avoid prolonged skin contact with cold objects or exposure to cold air temperatures. The first-line symptomatic therapy for ColdU is a non-sedating second-generation H1 antihistamine at approved doses for CSU. In patients who do not obtain complete control, increasing the dose up to 4 times approved doses for CSU is recommended. Steps 3 and 4 of treatment options include omalizumab and ciclosporin A, respectively. All of these treatments are used off-label and lack good evidence of effectiveness in patients with ColdU. Chronic inducible urticarias last several years and often pose a great treatment challenge because of their resistance to first-line therapy with H1-antihistamine. They are debilitating, often cause systemic reactions, including anaphylaxis in severe cases, and severely affect patient QoL. Avoidance of offending triggers poses massive changes to everyday life and therefore is typically not feasible. Thus, the need for approved treatment options for ColdU, especially for ColdU that have proven to be safe and efficacious in ColdU patients, is high.

ColdU presents as pruritic wheals with or without angioedema secondary to the release of mediators from mast cells after exposure of the skin to cold air, liquid, or cold objects. Degranulation of mast cells in ColdU is held to be mediated by Fc epsilon receptor (FcERI) activation, through cell surface-bound IgE cross-linked by as of yet unidentified auto-allergens. The subsequent release of histamine and other pro-inflammatory mediators leads to local tissue edema and pruritus. Many symptoms of urticaria are mediated primarily by the actions of histamine (a mast cell mediator) on the H1-receptors, and treatment with H1-antihistamine is a mainstay of therapy. Approximately 50% of patients achieve symptomatic control with conventional H1-antihistamine therapy. Increasing the dose of anti-histamines in some cases has been demonstrated to be effective but not in all cases.

### Study Overview

The EFC16720 study is a 24-week, randomized, double-blind, placebo-controlled, parallel-group, multi-center study to evaluate the use of dupilumab in adults and adolescents (≥12 to <18 years old) with primary acquired ColdU who remain symptomatic despite the use of H1-antihistamine. ColdU signs and symptoms are evaluated after a provocation test by the investigator (hives/wheals intensity) and participant (itch severity, skin pain, skin burning sensation). In addition, ColdU disease activity is assessed daily by the participant using the Cold Urticaria Activity Score (ColdUAS) questionnaire in an e-diary where the participant will report his/her skin reactions (wheals and swelling), skin sensations (itching, burning, pain or feeling hot), if they have been in contact with cold temperatures that usually cause skin reactions, if they have avoided trigger exposure, and overall symptoms severity. The study also assesses the effect of dupilumab on urticaria control, participants' health-related QoL and overall health status, proportion of patients with cold urticaria requiring emergency medical care visit or treatment with epinephrine and on reduction of rescue therapy.

The total anticipated number of participants randomized in the study is 60. This corresponds to approximately 30 participants who will be randomly assigned to each intervention arm: the dupilumab or the placebo treatment arm. At least 4 participants randomized in the study will be adolescents (≥12 to <18 years old) who will be recruited in a few selected countries. Randomization will be stratified by age (adolescent versus adult) and within adult group by country and by prior medical history of cold exposure triggered anaphylaxis or oropharyngeal edema; or urticaria requiring emergency medical care visit or treatment with epinephrine.

Participants who satisfy the inclusion and exclusion criteria will be randomized (1:1) to 1 of the following investigational medicinal product (IMP) treatment groups:
Dupilumab: 300 mg every 2 weeks (q2w) for adults; 200 mg q2w for adolescents ≥30 kg and <60 kg at baseline or 300 mg q2w for adolescents ≥60 kg at baseline.
Matched placebo.

### Duration of Study Period (Per Participant)

Screening period (2 to 4 weeks).
Randomized IMP treatment period (24 weeks).
Post IMP treatment period (12 weeks).

### Study Interventions

### Investigational Medicinal Product

Dupilumab 300 mg and placebo matching dupilumab 300 mg supplied in prefilled syringes that are visually indistinguishable.
Dupilumab 200 mg and placebo matching dupilumab 200 mg supplied in prefilled syringes that are visually indistinguishable.

### Objectives

### Primary

To demonstrate the efficacy of dupilumab in adult and adolescent participants with primary acquired cold urticaria (ColdU) who remain symptomatic despite the use of an H1-antihistamine.

### Secondary

To demonstrate the efficacy of dupilumab on primary acquired ColdU local signs and symptoms (hives/wheals, itch, burning sensation, and pain) after provocation test.

To demonstrate the efficacy of dupilumab on primary acquired ColdU disease activity.

To demonstrate the efficacy of dupilumab on primary acquired ColdU disease control.

To demonstrate improvement in health-related quality-of-life and overall disease status and severity.

To evaluate the ability of dupilumab in reducing the proportion of participants who require rescue therapy.

To evaluate the proportion of participants with cold exposure triggered urticaria.

To evaluate safety outcome measures.

To evaluate immunogenicity of dupilumab.

### Tertiary/Exploratory

To demonstrate the efficacy of dupilumab on disease activity over time. To demonstrate improvement in health-related quality-of-life specifically related to primary acquired ColdU. To evaluate the effect of dupilumab on general health status, healthcare resource utilization (HCRU), and productivity.

Pharmacokinetic (PK)/Pharmacodynamic (PD): to evaluate PK of dupilumab; and to evaluate PD effect of dupilumab.

### Endpoints

### Primary

Proportion of participants with negative ice cube provocation test at week 24 compared with placebo. A negative ice cube provocation test is defined as the absence of a confluent hive/wheal at the entire skin site of exposure after an ice cube provocation test. Provocation test reading time for all endpoints is 15 minutes after the ice cube application start, which is 5 minutes ice cube application plus 10 minutes of rewarming after removal of ice cube.

### Secondary

Change from baseline in local wheal intensity at the provocation site at week 12 and week 24 using the wheal intensity Likert scale ranging from 0 to 5 (clinician evaluation) compared with placebo. Change from baseline in local itch severity at the provocation site at week 12 and week 24 using the Peak Pruritus Numerical Rating Scale (NRS, score 0 to 10) (patient reported) compared with placebo. Change from baseline in local skin burning sensation at the provocation site at week 12 and week 24 using the peak burning sensation NRS (patient reported) compared with placebo. Change from baseline in local pain severity at the provocation site at week 12 and week 24 using the peak pain sensation NRS (patient reported) compared with placebo. Proportion of participants with negative ice cube provocation test at week 12 compared with placebo.

Change in proportion of participants with cold exposure triggered urticaria signs and symptoms from baseline to week 12 and week 24 as measured by ColdUAS7 compared with placebo. Change in proportion of days with cold exposure triggered urticaria signs and symptoms from baseline to week 12 and week 24 as measured by ColdUAS7 compared with placebo. Change in proportion of participants with severe cold exposure triggered urticaria signs and symptoms from baseline to week 12 and week 24 as measured by ColdUAS7 compared with placebo. Change in proportion of days with severe cold exposure triggered urticaria signs and symptoms from baseline to week 12 and week 24 as measured by ColdUAS7 compared with placebo.

Change from baseline in urticaria control test (UCT 4-item) at week 24 compared with placebo. Proportion of well-controlled participants (UCT≥12) at week 24 compared with placebo. Proportion of participants with an improvement of ≥3 in UCT-4 item from baseline to Week 24 compared with placebo.

Change from baseline in health-related quality-of-life (HRQOL) as measured by Dermatology Life Quality Index (DLQI) in patients ≥16 years old, and in Children's Dermatology Life Quality Index (CDLQI) in participants ≥12 to <16 years old at Week 24 compared with placebo. Patient Global Impression of Change (PGIC) of primary acquired ColdU at week 12 and week 24 compared with placebo. Change from baseline in Patient Global Impression of Severity (PGIS) of primary acquired ColdU at week 12 and week 24 compared with placebo.

Time to first rescue therapy for primary acquired chronic induced ColdU during the planned treatment period compared with placebo. Proportion of participants receiving rescue therapy for primary acquired chronic induced ColdU during the planned treatment period compared with placebo.

Proportion of participants with cold exposure triggered urticaria requiring emergency medical care visit or treatment with epinephrine (at provocation test and/or at home).

Percentages of participants experiencing treatment emergent adverse events (TEAEs) or serious adverse events (SAEs).

Incidence of treatment-emergent antidrug antibodies (ADA) against dupilumab over time.

### Tertiary/Exploratory

Change from baseline in ColdUAS7 at week 12 and at week 24 compared with placebo. Change from baseline in ColdUAS7 over time up to week 24 compared to placebo. Change from baseline in HRQOL as measured by Cold Urticaria Quality of Life (ColdU-QoL) questionnaire at week 24 compared with placebo. Change from baseline in 5-level version of the EuroQol-5D-5L version (EQ-5D-5L) questionnaire at week 24 compared with placebo. Cumulative number of HCRU compared with placebo.

Functional dupilumab concentrations in serum and PK profile. Total immunoglobulin E over time.

### Overall Study Design

The EFC16720 study is a 24-week, randomized, double-blind, placebo-controlled, parallel-group, multi-center study to evaluate the use of dupilumab in adults and adolescents (≥12 to <18 years old) with primary acquired ColdU who remain symptomatic despite the use of H1-antihistamine. These patients have failed antihistamine therapy and have active disease. The study is designed to test the hypothesis that dupilumab will increase the proportion of participants with negative ice cube provocation test compared with placebo.

A negative ice cube provocation test is defined as absence of a confluent hive/wheal at the entire skin site of exposure after ice cube provocation test.

ColdU signs and symptoms will be evaluated after a provocation test by the Investigator (hives/wheals intensity) and participant (itch severity, skin pain, skin burning sensation). In addition, ColdU disease activity will be assessed daily by the participant using the Cold Urticaria Activity Score (ColdUAS) questionnaire in an e-diary where the participant will report his/her skin reactions (wheals and swelling), skin sensations (itching, burning, pain or feeling hot), if they have been in contact with cold temperatures that usually cause skin reactions, if they have avoided trigger exposure, and overall symptoms severity. The study will also assess the effect of dupilumab on urticaria control, participants' health-related QOL and overall health status, proportion of patients with cold urticaria requiring emergency medical care visit or treatment with epinephrine and on reduction of rescue therapy.

### Scientific Rationale for Study Design

In the EFC16720 study, the target population consists of patients with primary acquired ColdU who remain symptomatic despite treatment with H1-antihistamine alone as these patients have a significant unmet medical need. Therapy for the patients is focused on the avoidance of the trigger factor and symptomatic treatment. The updated international guideline on the definition, classification, diagnosis and management of urticaria provides recommendations and a treatment algorithm. The consensus recommendations for ColdU management are mainly based on treatments approved for CSU, and some clinical data available in patients suffering from various types of ColdUs (cholinergic, symptomatic dermographism, ColdU). The recommended therapies are, in general, not approved for ColdU by regulatory agencies, with only limited specific countries where antihistamines are approved under broader “urticaria” indications. A similar stepwise approach as in patients with CSU is recommended for ColdU, including ColdU. Steps 1 and 2 of this algorithm is the use of non-sedating H1-antihistamines at approved, or increased doses (up to 4-fold), respectively. Steps 3 and 4 of treatment options include omalizumab and ciclosporin A, respectively. More than 50% of acquired primary ColdU patients do not respond to H1 antihistamine treatment. Omalizumab, a monoclonal anti-immunoglobulin E (IgE) antibody, is the most used off-label treatment in antihistamine-resistant patients with ColdU including ColdU. However, approximately a third of the patients treated with omalizumab are not well-controlled.

Study EFC16720 targets patients not adequately controlled with H1 antihistamine treatment and allows the use of H1-antihistamine at up to 4-fold the approved doses as background medication at stable doses.

There are a limited number of studies conducted in patients with cold urticaria. As mentioned above, the guideline recommendations for CSU are adopted for patients with ColdU; however there are several differences between CSU and ColdU. In ColdU, the symptoms only occur after skin exposure to physical or chemical triggers. The duration of individual wheals is often relatively brief for ColdU, lasting minutes to hours. This is the reason why different assessment tools and endpoints are used in ColdU clinical studies compared with CSU. Cold provocation tests are used in clinical studies in patients with chronic urticaria to evaluate efficacy of treatments, mainly antihistamine treatments, but also doxepin, cyproheptadine, either by evaluating proportion of patients who after treatment did not develop signs/symptoms with the cold provocation test or evaluating the response to an experimental cold-simulation time test (CSTT) i.e., minimum time threshold of cold stimulation required to induce a coalescent wheal and other cold urticaria signs and symptoms. This study will assess efficacy using the primary endpoint of the proportion of participants with negative ice cube provocation test at week 24. The patients/Investigators will also score local signs and symptoms before and after provocation test assessing effect of the IMP on hives, itch, burning sensation and pain. Ice cube provocation test is a traditional test used in common practice. For the study, the method will be standardized and compliant with according to the International EAACI/GA2LEN/EDF/UNEV guidelines. In addition to provocation test, cold urticaria signs and symptoms will be evaluated using ColdUAS. It is scored by patients daily to assess overall disease activity.

A large portion of patients with ColdU (35% to 70%) have experienced systemic reactions including anaphylaxis, after extensive cold exposure and exercise, respectively. Avoidance of offending triggers poses massive changes to everyday life and therefore is typically not feasible. This has significant impact on patients' QoL. The study will assess the proportion of patients with cold exposure triggered urticaria requiring emergency medical care visit or treatment with epinephrine urticaria.

### End of Study Definition

A participant is considered to have completed the study if he/she has completed all phases of the study including the last end of study (EOS) visit. If a participant discontinues the treatment period prematurely but completes follow-up to the planned EOS visit, he/she is considered a completer. The overall EOS is defined as the date of the last visit of the last participant in the study.

### Inclusion Criteria

Participants are eligible to be included in the study only if all of the following criteria apply:

### Age

Participant must be ≥12 years to 80 years (or the minimum legal age for adolescents in the country of the investigational site) of age inclusive at the time of signing the informed consent. For those countries where local regulations do not permit enrollment of adolescents (≥12 years to <18 years of age), the recruitment will be restricted to those who are ≥18 years of age.

### Type of Participant and Disease Characteristics

Participants who have a diagnosis of primary acquired ColdU defined as recurrence of itchy wheals and/or angioedema due to cold for longer than 6 weeks prior to screening visit (visit 1).

Participants with positive ice cube provocation test, i.e., presenting at least a confluent hive/wheal on the exposed skin area, at the screening visit (visit 1) and randomization visit (visit 2).

Participants meeting at least 1 of the following criteria despite use of H1-AH: Urticaria Control Test (UCT) (4-item)<12 at the screening visit (visit 1) and randomization visit (visit 2); within 6 months prior to the screening visit, documented medical history of cold exposure triggered anaphylaxis or oropharyngeal edema; within 6 months prior to the screening visit, documented medical history of cold exposure triggered urticaria requiring emergency medical care visit or treatment with epinephrine.

Participants using a study-defined H1-antihistamine for primary acquired ColdU. Note: Participants should remain on their prescreening non-sedating H1-antihistamine dose. Up to 4-fold the recommended dose is allowed. If participants are on dose higher than 4-fold the recommended dose at screening, the Investigator can adjust the participant dose to the stipulated range at the screening visit (visit 1), if clinically appropriate. The H1-antihistamine dose should be stable for at least 3 consecutive days prior to the screening visit (visit 1).

### Weight and Sex

Body weight ≥30 kg, and male or female

### Female Participants

Contraceptive use by women should be consistent with local regulations regarding the methods of contraception for those participating in clinical studies. A female participant is eligible to participate if she is not pregnant or breastfeeding, and at least 1 of the following conditions applies: is not a woman of childbearing potential (WOCBP); or is a WOCBP and agrees to use a contraceptive method that is highly effective, with a failure rate of <1%. A WOCBP must have a negative highly sensitive pregnancy test (urine or serum as required by local regulations) on day 1 before the first dose of study intervention. If a urine test on day 1 cannot be confirmed as negative (e.g., an ambiguous result), a serum pregnancy test is required. In such cases, the participant must be excluded from participation if the serum pregnancy result is positive.

### Informed Consent

Capable of giving signed informed consent which includes compliance with the requirements and restrictions listed in the informed consent form (ICF) and in this protocol. In countries where the legal age of majority is above 18 years old, a specific ICF must also be signed by the participant's legally authorized representative. For adolescents, both the adolescent and the parent/legally authorized representative must sign the specific ICF.

### Exclusion Criteria

Participants are excluded from the study if any of the following criteria apply:

### Medical Conditions

Clearly defined underlying etiology for urticaria other than primary acquired ColdU. This includes but is not limited to the following urticarias: acute urticaria; chronic spontaneous urticaria; inducible urticaria: all other forms of ColdU (acquired secondary ColdU, atypical acquired ColdU, hereditary ColdU syndromes), solar, cholinergic, heat, aquagenic, vibratory angioedema, symptomatic dermographism, delayed pressure, or contact; diseases with possible symptoms of urticaria or angioedema: systemic lupus erythematosus, urticarial vasculitis, urticaria pigmentosa, erythema multiforme, mastocytosis, hereditary or acquired angioedema, lymphoma, leukemia, or generalized cancer.

Systemic hypersensitivity reaction including anaphylaxis related or suspected to be related to ice cube provocation test at the screening visit (visit 1) and randomization visit (visit 2).

Presence of skin morbidities or associated with itch other than primary acquired ColdU that may interfere with the assessment of the study outcomes.

Participants having active atopic dermatitis.

Diagnosed active endoparasitic infections; suspected or high risk of endoparasitic infection, unless clinical and (if necessary) laboratory assessment have ruled out active infection before randomization.

History of human immunodeficiency virus (HIV) infection or positive HIV 1/2 serology at the screening visit (Visit 1).

Severe concomitant illness(es) that, in the Investigator's judgment, would adversely affect he participant's participation in the study. Examples include, but are not limited to participants with short life expectancy, participants with uncontrolled diabetes (hemoglobin A1c ≥9%), participants with cardiovascular conditions (e.g., Class III or IV cardiac failure according to the New York Heart Association classification), severe renal conditions (e.g., participants on dialysis), hepato-biliary conditions (e.g., Child-Pugh class B or C), neurological conditions (e.g., demyelinating diseases), active major autoimmune diseases (e.g., lupus, inflammatory bowel disease, rheumatoid arthritis, etc.), other severe endocrinological, gastrointestinal, metabolic, pulmonary, or lymphatic diseases. The specific justification for participants excluded under this criterion will be noted in study documents (chart notes, case report forms (CRFs), etc.).

Known or suspected immunodeficiency, including history of invasive opportunistic infections (eg, tuberculosis (TB), histoplasmosis, listeriosis, coccidioidomycosis, pneumocystosis, and aspergillosis) despite infection resolution, or otherwise recurrent infections of abnormal frequency or prolonged duration suggesting an immune-compromised status, as judged by the Investigator.

Participants with active TB or nontuberculous mycobacterial infection, or a history of incompletely treated TB will be excluded from the study unless it is well-documented by a specialist that the participant has been adequately treated and can now start treatment with a biologic agent, in the medical judgment of the Investigator and/or infectious disease specialist. Tuberculosis testing will be performed on a country by country basis, according to local guidelines if required by regulatory authorities or ethics boards.

Active chronic or acute infection requiring treatment with systemic antibiotics, antivirals, antiprotozoals, or antifungals within 2 weeks before the screening visit and during the screening period.

History of malignancy within 5 years before Visit 1, except completely treated in situ carcinoma of the cervix, completely treated and resolved nonmetastatic squamous or basal cell carcinoma of the skin.

Known or suspected alcohol and/or drug abuse.

History of systemic hypersensitivity or anaphylaxis to any other biologic therapy or any of its excipients.

Planned major surgical procedure during the participant's participation in this study.

Participants with any other medical or psychological condition including relevant laboratory or electrocardiogram abnormalities at screening that, in the opinion of the Investigator, suggest a new and/or insufficiently understood disease, may present an unreasonable risk to the study participant as a result of his/her participation in this clinical study, may make participant's participation unreliable, or may interfere with study assessments. The specific justification for participants excluded under this criterion will be noted in study documents (chart notes, CRF, etc.).

### Prior/Concomitant Therapy

Participation in a prior dupilumab clinical study or have been treated with commercially available dupilumab.

Exposure to another systemic or topical investigative drug (monoclonal antibodies as well as small molecules) within a certain time period prior to the screening visit (visit 1), defined as follows: an interval of <6 months or <5 PK half-lives for investigative monoclonal antibodies, whichever is longer, and an interval of <30 days or <5 PK half-lives, whichever is longer, for investigative small molecules.

Having used any of the following treatments within 4 weeks before the screening visit (visit 1): immunosuppressive/immunomodulating drugs (e.g., systemic corticosteroids (oral or parenteral-intravenous, intramuscular, SC)), cyclosporine, mycophenolate-mofetil, interferon gamma, Janus kinase inhibitors, azathioprine, methotrexate, hydroxychloroquine, sulfasalazine, dapsone, colchicine, etc.); antifibrinolytic tranexamic acid and epsilon-aminocaproic acid; leukotriene receptor antagonists (LTRAs) and H2 receptor antagonists. Note: patients taking stable LTRAs and/or H2 receptor antagonists for diseases other than CSU (e.g., asthma or gastroesophageal reflux disease, respectively) will be permitted to continue their use; phototherapy, including tanning beds.

Treatment with biologics as follows: any cell-depleting agents including but not limited to rituximab: within 6 months before the screening visit (visit 1); anti-immunoglobulin E therapy (omalizumab) within 4 months before the screening visit (visit 1); other monoclonal antibodies (which are biological response modifiers): within 5 half-lives (if known) or 16 weeks before the screening visit (visit 1), whichever is longer.

Treatment with a live (attenuated) vaccine within 4 weeks before the screening visit (visit 1). Note: For participants who have vaccination with live, attenuated vaccines planned during the course of the study (based on national vaccination schedule/local guidelines), it will be determined, after consultation with a physician, whether the administration of vaccine can be postponed until after the EOS, or preponed to before the start of the study without compromising the health of the participant: participants for whom administration of live (attenuated) vaccine can be safely postponed would be eligible to enroll into the study; participants who have their vaccination preponed can enroll in the study only after a gap of 4 weeks following administration of the vaccine.

Routine (daily or every other day during 5 or more consecutive days) doses of doxepin within 14 days prior to the screening visit (visit 1).

Planned or anticipated use of any prohibited medications and procedures during the screening and study treatment period.

Either intravenous immunoglobulin (IVIG) therapy and/or plasmapheresis within 30 days prior to the screening visit (Visit 1).

### Diagnostic Assessments

Participants with any of the following result at the screening visit (Visit 1): positive (or indeterminate) hepatitis B virus surface antigen (HBs Ag) HBs Ag or; positive total hepatitis B core antibody (HBc Ab) confirmed by positive hepatitis B virus (HBV) Deoxyribonucleic acid (DNA) or; positive hepatitis C antibody (HCV Ab) confirmed by positive hepatitis C virus (HCV) ribonucleic acid (RNA).

Non-Compliance of the e-Diary

Participants are not complaint with completion of e-diary by completing entries on less than 4 days out of the 7 days immediately preceding the baseline visit (visit 2).

### Other Exclusions

Individuals accommodated in an institution because of regulatory or legal order, prisoners or participants who are legally institutionalized.

Any country-related specific regulation that would prevent the participant from entering this study.

Participant not suitable for participation, whatever the reason, as judged by the Investigator, including medical or clinical conditions, or participants potentially at risk of noncompliance to study procedures.

Participants are employees of the clinical study center or other individuals directly involved in the conduct of the study, or immediate family members of such individuals.

Any specific situation during study implementation/course that may raise ethical concerns.

Sensitivity to any of the study interventions, or components thereof, or drug or other allergy that, in the opinion of the Investigator, contraindicates participation in the study.

### Lifestyle Considerations

Participants unwilling to attempt avoidance of known cold triggers.

### Screen Failures

Screen failures are defined as participants who consent to participate in the clinical study but are not subsequently randomly assigned to study intervention/entered in the study. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure reasons, eligibility criteria, and any SAE.

Individuals who do not meet the criteria for participation in this study (screen failure) may be rescreened once. Rescreened participants will be assigned a new participant number versus the 1 received for the initial screening visit (visit 1).

### Study Interventions

### Investigational Medicinal Product

Dupilumab 300 mg and placebo matching dupilumab 300 mg supplied in prefilled syringes that are visually indistinguishable. Dupilumab 200 mg and placebo matching dupilumab 200 mg supplied in prefilled syringes that are visually indistinguishable.

### Dupilumab Formulation

Dupilumab 300 mg: a 150 mg/mL dupilumab solution in a pre-filled syringe to deliver 300 mg in a 2 mL injection. Dupilumab 200 mg: a 175 mg/mL dupilumab solution in a pre-filled syringe to deliver 200 mg in a 1.14 mL injection.

### Dose Regimen

One subcutaneous (SC) injection of 300 mg q2w after an initial SC loading dose of 600 mg (2 injections of 300 mg) on Day 1 for all adult participants and for adolescents weighing ≥60 kg. One SC injection of 200 mg q2w after an initial SC loading dose of 400 mg (2 injections of 200 mg) in adolescent participants ≥30 kg and <60 kg.

### Placebo

### Formulation

Placebo matching dupilumab 300 mg: identical formulation to the active 300 mg formulation without dupilumab, in a pre-filled syringe to deliver placebo in a 2 mL injection or, placebo matching dupilumab 200 mg: identical formulation to the active 200 mg formulation without dupilumab, in a pre-filled syringe to deliver placebo in a 1.14 mL injection.

### Dose Regimen

One SC injection of placebo matching 300 mg q2w after an initial matching placebo loading dose of 600 mg (2 injections of placebo matching 300 mg) on Day 1 for all adult participants and for adolescents weighing ≥60 kg. One SC injection of placebo matching 200 mg q2w after an initial matching placebo loading dose of 400 mg (2 injections of placebo matching 200 mg) in adolescent participants ≥30 kg and <60 kg.

### Packaging and Labeling

Each dose of dupilumab will be supplied as 1 glass pre-filled syringe packed in a participant kit box. Both glass pre-filled syringe and box will be labeled as required per country requirement. Each dose of placebo will be supplied as 1 glass pre-filled syringe packed in a participant kit box. Both glass pre-filled syringe and box will be labeled as required per country requirement.

The IMP is administered every 14±3 days (q2w) during the 24-week treatment period with the last IMP administration at week 22.

The first IMP administration should be performed at the study center. Subsequent IMP administrations can be done at home by the participant (or parent/legally authorized representative, or caregiver). If the participant (or parent/legally authorized representative or caregiver) is unable or unwilling to prepare and inject IMP, injections can be performed at the study center by way of unscheduled visits; or arrangements can be made for qualified study center personnel and/or health care professionals (eg, visiting nurse service) to administer IMP at participant's home.

For doses not given at the study center, paper diaries will be provided to record information related to the injections. The paper diary will be kept as source data in the participants study file.

At visit 2, the Investigator or delegate will prepare and inject the first dose of IMP in front of the participant (or parent/legally authorized representative, or caregiver). If home administration is planned, the participant (or parent/legally authorized representative or caregiver) will prepare and inject the second dose of IMP under the supervision of the Investigator or delegate. The training must be documented in the participant's study file. In case of emergency (e.g., natural disaster, pandemic etc.) different training ways (e.g., virtual training via video call etc.) can be performed (and will be documented in the participant's study file).

Subcutaneous injection sites should alternate between the upper thighs, 4 quadrants of the abdomen or the upper arms, so that the same site is not injected twice during consecutive administrations. Injection in the upper arms can only be done by a trained person (parent/legally authorized representative/caregiver trained by the Investigator or Delegate) or health care professional but not the participants themselves.

Participants should be monitored for at least 30 minutes. The monitoring period may be extended as per country specific or local study center-specific requirements.

The participant/parent/legally authorized representative/caregiver should be trained by the study center staff to recognize potential signs and symptoms of hypersensitivity reaction in order to self-monitor/monitor at home for at least 30 minutes (or longer per country-specific or local site-specific requirements) following injection. In case of hypersensitivity symptom/s the participant should contact his/her healthcare provider/emergency contact.

The following contingencies may be implemented for the duration of the emergency (after Sponsor agreement is obtained) to make clinical supplies available to the participant for the duration of the emergency: in case of emergency (e.g., natural disaster, pandemic, etc.), IMP may be supplied from the study center to the participant via a Sponsor-approved courier company where allowed by local regulations and approved by the participant (or parent/legally authorized representative); when the participant has a study visit, the IMP will be administered following clinical procedures and blood collection.

### Non-Investigational Medicinal Product(s)

Participants should continue their established standard of care background therapy with a long acting non-sedating H1-antihistamine, at up to 4-fold the recommended dose for. If participants are on a dose higher than 4-fold the recommended dose at the screening visit (visit 1), the Investigator can adjust the participant's dose within the stipulated range at the screening visit (visit 1). Participants should continue to take the same daily dose throughout the study unless they experience a flare, for which rescue therapy may be initiated. The following list of H1-antihistamines are allowed and noted with their recommended dose: Cetirizine 10 mg once per day (qd); Levocetirizine dihydrochloride 5 mg qd; Ebastine 10 mg qd; Fexofenadine 60 mg twice per day or 180 mg qd; Loratadine 10 mg qd; Desloratadine 5 mg qd; Bilastine 20 mg qd; Rupatadine 10 mg qd; Other H1-antihistamines after discussion with the Sponsor.

### Methods of Assigning Patients to Treatment Group

The randomized intervention kit number list is generated centrally by Sanofi and IMPs (dupilumab 300 mg, dupilumab 200 mg, or their matching placebo) are packaged in accordance with this list. The randomization and intervention allocation are performed centrally by an Interactive Response Technology (IRT). The IRT generates the participant randomization list and allocates the intervention number and the corresponding intervention kits to the participants according to it.

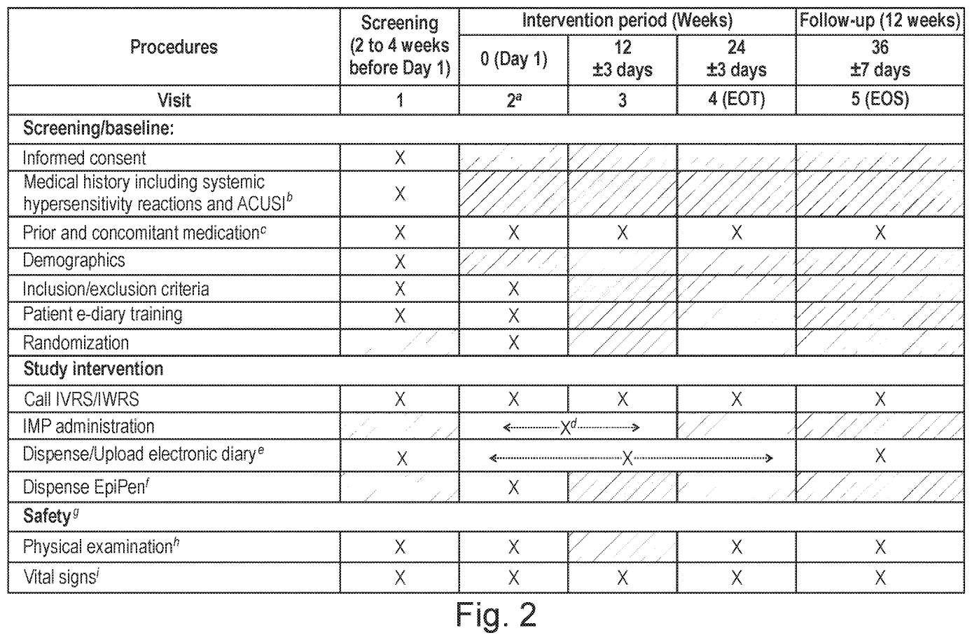

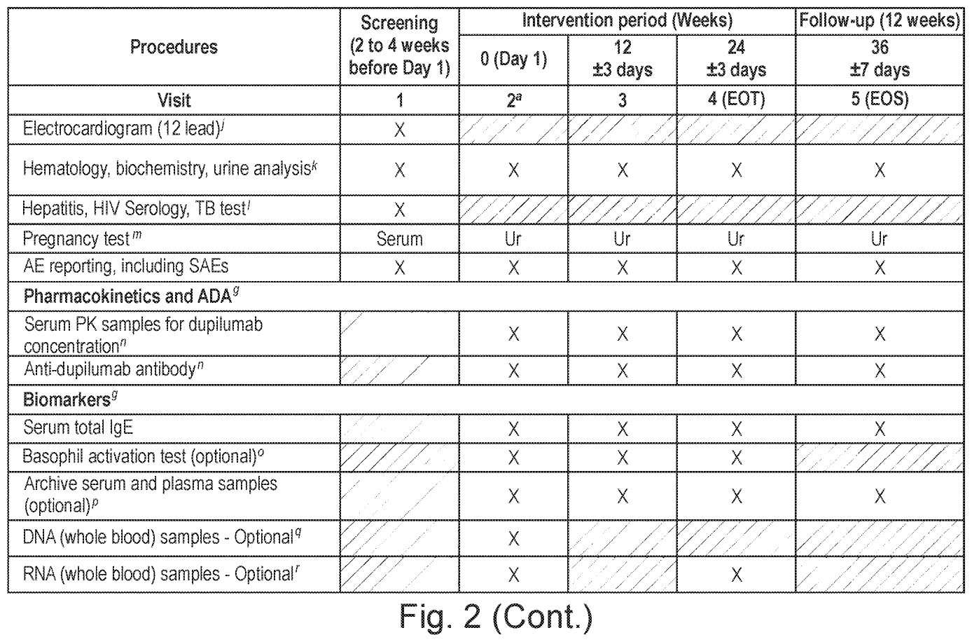

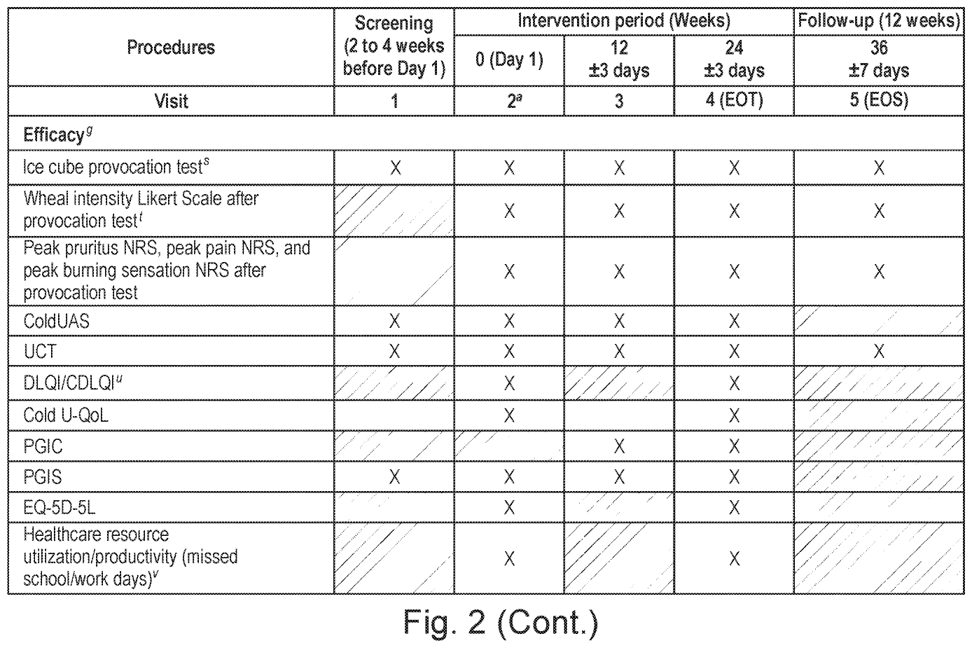

Study intervention will be dispensed at the study visits summarized in schedule of activities (SoA). (See FIG. 2.) Returned study intervention should not be re-dispensed to the participants.

FIG. 2: ^{a}Randomization/baseline Visit is defined as Day 1. All assessments at Visit 2 (Day 1) are to be conducted pre-IMP dose with the exception of the assessment of local tolerability of SC injections. ^{b}ACUSI will be captured in the eCRF. ^{c}Concomitant medication, including rescue OCS taken since last visit will be collected throughout the study. ^{d}Loading dose at Day 1 (Visit 2): 600 mg/matched placebo (2 SC injections) for 300 mg/matched placebo every 2 weeks (q2w) regimen for adults and adolescents ≥60 kg OR 400 mg/matched placebo (2 SC injections) for 200 mg/matched placebo q2w regimen for adolescents ≥30 kg and <60 kg. Investigational medicinal product will be administered every other week. The planned last dose is at Week 22. Participants are allowed to self-inject IMP at home after appropriate training of the participants (or parent/legally authorized representatives, or caregivers). ^{e}Electronic diary is used for daily recording of ColdUAS and antihistamine medication from screening up to Week 24. This device is dispensed at screening visit (visit 1), including instructions for use. At the EOT, the e-diary is returned to the study center. For UCT, DLQI (>16 years old)/CDLQI (<16 years old), ColdQoL and EQ-5D-5L the participant will fill in the questionnaires during their study center visit in the e-diary. The e-diary will be also used to complete peak Pruritus NRS, peak pain NRS, and peak burning sensation NRS, after the provocation test. Participants will complete the PGIS and PGIC on the e-diary after they have answered the peak pruritus NRS, peak pain NRS and peak burning sensation NRS. ^{f}EpiPen or equivalent to be provided locally. ^{g}Assessments/procedures should be conducted in the following order: patient-reported outcomes, Investigator assessments, safety and laboratory assessments (including sample collection for ADA, PK, biomarker, and optional DNA and RNA), ice cube provocation test, participant's and Investigator's assessment of signs and symptoms after provocation test, and administration of IMP. ^{h}Physical examinations will include skin, nasal cavities, eyes, ears, respiratory, cardiovascular, gastrointestinal, neurological, lymphatic, and musculoskeletal systems. ⁱVital signs, including systolic and diastolic blood pressure (mmHg), pulse rate (beats per minute), body temperature (°C), and respiratory rate will be measured at every visit prior and after provocation test. Height (cm) will be measured at screening visit (Visit 1) only. Body weight (kg) will be measured at screening visit (Visit 1) and at EOT/EOS visits. ^{j}ECG to be locally collected and read. ^{k}Hematology will include hemoglobin, hematocrit, platelet count, total white blood cell count, differential count, and total red blood cell count. Serum chemistry will include creatinine, blood urea nitrogen, glucose, lactate dehydrogenase, uric acid, total cholesterol, total protein, albumin, total bilirubin, alanine aminotransferase, aspartate aminotransferase, alkaline phosphatase, electrolytes (sodium, potassium, chloride), bicarbonate, and creatine phosphokinase. Urinalysis will include specific gravity, pH, glucose, ketones, blood, protein, nitrate, leukocyte esterase, urobilinogen, and bilirubin. In case the urine dipstick test result is abnormal, a urine sample should be sent into the central laboratory for microscopic examination. ^{l}Clinical laboratory testing at screening visit (visit 1) will include hepatitis screen covering HBs Ag, HBs Ab, HBc Ab, HCV Ab, HIV screen (Anti-HIV-1 and HIV-2 antibodies). In case of results showing HBs Ag (negative) and HBc Ab (positive), an HBV DNA testing will be performed and should be confirmed negative prior to randomization. In case of results showing HCV Ab (positive), an HCV RNA testing will be performed and should be confirmed negative prior to randomization. ^{m}Only for women of childbearing potential. Pregnancy will lead to definitive treatment discontinuation in all cases. Pregnancy testing should be done monthly, female participants will be supplied with dipsticks for months with no study center visits planned. In female participants who discontinue the study intervention, the pregnancy testing should continue for a minimum of 12 weeks after the last dose of study intervention. In between visit, urine pregnancy tests must be performed at home. ⁿSerum dupilumab concentration and ADA samples will be collected and archived prior to administration of IMP. In the event of any SAE, any AE of severe injection site reaction lasting longer than 24 hours, or any AESI of anaphylactic reaction or systemic allergic reaction that is related to IMP and require treatment, PK and ADA samples will be collected at or near the onset of the event for any additional analysis if required or for archival purposes. ^{o}For participants (with exception of adolescents) who decide to participate and provide a specific written informed consent for the optional basophil activation test. ^{p}For participants (with exception of adolescents) who decide to participate and provide a specific written informed consent for the optional archive serum and plasma sample. Archive serum and plasma samples (optional) are collected for future analysis of potential biomarkers of drug response, disease activity, safety and the type 2 inflammation pathway. ^{q}For participants (with exception of adolescents) who decide to participate and provide a specific written informed consent for the optional genomics sub-study (DNA sample collection). The DNA sample should be collected at the day 1 visit but can be collected at any visit during the study. ^{r}For participants (with exception of adolescents) who decide to participate and provide a specific written informed consent for the optional genomics sub-study (RNA sample collection). The RNA sample must be collected before the administration of the first dose of IMP and at Week 24 before the administration of the IMP. ^{s}Positive provocation test defined as presence of at least a confluent hive/wheal at the entire skin site of exposure after ice cube provocation test. ^{t}The Wheal intensity Likert Scale for wheal intensity after provocation test will be completed by the Investigator after the provocation test and entered in the eCRF. ^{u}Participants will complete the DLQI (≥16 years old) or CDLQI (≥12 to <16 years old). ^{v}HCRU (missed school/work days) baseline version to be administered at baseline; postbaseline version to be administered at the subsequent visits. It will be entered in the eCRF.

### Methods of Blinding

Dupilumab 300 mg/200 mg and placebo matching dupilumab 300 mg/200 mg will be provided in identically matched 2 mL/1.14 mL pre-filled syringes that are visually indistinguishable for each dose. Syringes and boxes will be labeled with a treatment kit number. Whilst this study is double-blinded in terms of treatment with either dupilumab or placebo, it is not blinded to weight-based dose levels, due to the different volume size (2 mL versus 1.14 mL) of the dose level of dupilumab (300 mg/matching placebo or 200 mg/matching placebo) that will be used for the different weight categories for adolescents.

### Concomitant Therapy

Any medication or vaccine (including over-the-counter or prescription medicines, vitamins, and/or herbal supplements) that the participant is receiving at the time of enrollment or receives during the study must be recorded along with: reason for use; dates of administration including start and end dates; dosage information including dose and frequency.

Non-sedating H1-antihistamines, at up to 4-fold the approved dose for CSU, are allowed as background medication and on demand as recue medication. See Section 6.1.2 and Section 6.5.1 for details.

The concomitant use of the following therapies is prohibited during the entire study. Study treatment will need to be discontinued in participants receiving these treatments: systemic immunosuppressants (immunosuppressive/immunomodulating drugs) e.g., systemic corticosteroids (oral or parenteral (intravenous, intramuscular, SC)), cyclosporine, mycophenolate-mofetil, interferon gamma, Janus kinase inhibitors, azathioprine, methotrexate, hydroxychloroquine, dapsone, sulfasalazine, colchicine, etc. Note: a short course of OCS is allowed as rescue therapy; any cell-depleting agents including but not limited to rituximab; monoclonal antibodies (which are biological response modifiers) including anti-immunoglobulin E therapy (omalizumab); treatment with a live (attenuated) vaccine; IVIG; plasmapheresis; other investigational drugs.

The concomitant use of following therapies is prohibited during the entire study but study treatment will not need to be discontinued in participants receiving these treatments in violation of the protocol: topical corticosteroids; topical calcineurin inhibitors; topical and oral antihistamines (other than those allowed as background therapy); routine doses of doxepin (daily or every other day during 5 or more consecutive days); LTRAs and H2 receptor antagonists, unless stable and taken for diseases other than ColdU; antifibrinolytic tranexamic acid and epsilon-aminocaproic acid; phototherapy, including tanning beds.

### Rescue Medicine

All participants on 1- to 3-fold the approved non-sedating H1-antihistamine dose (maintenance dose used at screening) will be allowed to take additional doses of their H1-antihistamine medications as rescue therapy as long as they do not exceed 4-fold the approved dose during the screening, treatment, and follow-up periods. If symptoms are still uncontrolled after increase of H1-antihistamine to the maximum allowed dose, or if the participant is already on the 4-fold approved H1-antihistamine, participants can switch to another antihistamines up to 4-fold the approved dose or a short course of OCS is allowed during the treatment and follow-up periods. In order to ensure consistency, when possible, it is recommended to use OCS for 5 to 7 days with a starting dose of oral prednisone 40 mg (or clinically comparable OCS) followed by taper per the Investigator's judgment.

In case of systemic hypersensitivity reactions due to cold, the participants may require epinephrine treatment. EpiPen (or a local equivalent) will be provided to the participants at the beginning of the study, and the participants should be appropriately trained how and when to use it. EpiPen use should be in accordance with the locally approved product labeling.

The initial maintenance antihistamine dose should remain stable throughout the study, and participants should continue their maintenance dose of initial H1-antihistamine once rescue treatment is no longer required. The use of permitted rescue medications should be delayed, if possible, for at least 8 weeks following the initiation of the investigational treatment. The date and time of rescue medication administration as well as the name and dosage regimen of the rescue medication must be recorded.

### Discontinuation of Study Intervention

In rare instances, it may be necessary for a participant to permanently discontinue study intervention. If study intervention is permanently discontinued, the participant should complete the early treatment discontinuation visit with all assessments planned for the end of treatment (EOT) visit. See FIG. 2 for data to be collected at the time of discontinuation of study intervention.

The participants may withdraw from treatment with the IMP if he or she decides to do so, at any time and irrespective of the reason, or this may be the Investigator's decision. All efforts should be made to document the reason(s) for treatment discontinuation and this should be documented in the eCRF.

Participants must be permanently withdrawn from the study treatment for the following reasons: at their own request or at the request of their legally authorized representative (legally authorized representative means an individual or judicial or other body authorized under applicable law to consent on behalf of a prospective participant to the patient's participation in the procedure(s) involved in the research); if, in the Investigator's opinion, continuation in the study would be detrimental to the participant's well-being at the specific request of the Sponsor; in the event of a protocol deviation, at the discretion of the Investigator or the Sponsor; any code broken requested by the Investigator will lead to permanent discontinuation of study intervention; pregnancy; anaphylactic reactions or systemic allergic reactions that are related to IMP and require treatment; diagnosis of a malignancy during study, excluding carcinoma in situ of the cervix, or squamous or basal cell carcinoma of the skin; any opportunistic infection or other infections whose nature or course may suggest an immunocompromised status; serum alanine aminotransferase (ALT)>3×upper limit of normal (ULN) and total bilirubin >2×ULN; Serum ALT >5×ULN if baseline ALT≤2×ULN or ALT >8×ULN if baseline ALT >2×ULN; if the participant develops a medical condition that requires use of prohibited medication; if the participant misses more than 2 consecutive doses of the IMP; Systemic hypersensitivity reaction including anaphylaxis related or suspected to be related to ice cube provocation test. In the participants who experienced such reaction no further ice cube provocation test should be done during the study.

Any abnormal laboratory value or ECG parameter will be immediately rechecked for confirmation within a reasonable timeframe as assessed by the Investigator before making a decision of definitive discontinuation of the IMP for the concerned participant.

If a clinically significant ECG finding is identified (including, but not limited to changes from baseline in QT interval corrected using (Bazett's formula (QTcB) or Fridericia's formula (QTcF))) at screening, the Investigator or qualified designee will determine if the participant is eligible for the study. This review of the ECG printed at the time of collection must be documented. Any new clinically relevant finding should be reported as an AE.

Participants will be followed-up according to the study procedures specified in this protocol up to the scheduled date of study completion, or up to recovery or stabilization of any AE to be followed-up as specified in this protocol, whichever comes last.

Participants who discontinue the study intervention prematurely (prior to completing the 24-week treatment period) will perform, as soon as possible, the early treatment discontinuation visit with all assessments normally planned for the EOT visit (visit 4), to assure a complete clinical assessment in close temporal proximity to the premature termination of study treatment is available.

In addition, and to allow assessment of participant outcomes over the stipulated study period, participants will be asked and encouraged to complete all remaining study treatment visits, and participate in safety follow-up according to the visit schedule with a +3 day window. Under exceptional circumstances when a participant cannot come to the study center for a scheduled visit, a phone contact can be made. During the phone contact, at least information about AEs, concomitant medication and status of urticaria should be collected.

Temporary intervention discontinuation may be considered by the Investigator because of suspected AEs.

In addition, if patients become infected while receiving treatment with dupilumab and do not respond to anthelminthic treatment, treatment with dupilumab should be temporarily discontinued until infection resolves.

If the participant misses more than 2 consecutive doses, the participant will be permanently discontinued from the study treatment.

For all temporary intervention discontinuations, duration should be recorded by the Investigator in the appropriate pages of the eCRF.

Reinitiation of intervention with the IMP will be done under close and appropriate clinical/and or laboratory monitoring once the Investigator will have considered according to his/her best medical judgment that the responsibility of the IMP(s) in the occurrence of the concerned event was unlikely and if the selection criteria for the study are still met.

### Efficacy Assessments

The e-diary is used for: daily recording of the ColdUAS questionnaire; daily recording of H1-antihistamine medication use; DLQI (≥16 years old)/CDLQI (≥12 to <16 years old), UCT, ColdU-QOL and EQ-5D-5L evaluation at the study center visit; assessment of peak pruritus NRS, peak burning sensation NRS, peak pain sensation NRS after provocation test; assessment of PGIS and PGIC after participants have completed the NRS.

Timing of the assessment is specified in FIG. 2.

This device will be dispensed at the screening visit (visit 1), including instructions for use and participants will be instructed on the use of the device.

Recorded information will be downloaded from this device daily. At the EOT visit, the e-diary will be downloaded and returned to the study center.

The study center staff regularly, and when alerts received, should check the status of the compliance to background therapy and overall e-diary compliance including ColdUAS. The study center should follow-up with the participant as appropriate.

Wheal intensity Likert scale and missed school/work day questionnaire will be filled in by the Investigator/study center staff in the eCRF.

### Various Testing Types

### Ice Cube Provocation Test

Cold urticaria is defined by the appearance of its signs and symptoms (hives/wheals, itch, pain, and burning sensation) after contact cooling and rewarming of the skin. Chronic inducible urticarias, including primary acquired ColdU, are diagnosed based on the patient history and the results of provocation testing. Moreover, cold provocation tests are used in clinical studies in patients with chronic urticaria to evaluate efficacy of treatments, mainly antihistamine treatments, but also doxepin, cyproheptadine, either by evaluating the proportion of patients who after treatment did not develop signs/symptoms with the cold provocation test or evaluating the response to an experimental CSTT, i.e., minimum time threshold of cold stimulation required to induce a coalescent wheal and other ColdU signs and symptoms.

The ice cube provocation test is the most frequently used provocation method for ColdU in routine clinical practice and is therefore proposed for the present clinical study.

The consensus recommendations for ColdU (EAACI/GA²LEN/EDF/UNEV) defines the following for provocation methods for ColdU: provocation tests should be performed by applying a cold stimulus to forearm skin; old provocation testing should be performed for 5 minutes on volar forearm; ice cube should be melting within a thin plastic bag to avoid cold damage of the skin and to prevent direct water contact to avoid the confusion with aquagenic urticaria if the test is positive; reading time 10 minutes of rewarming after ice cube is removed.

Emergency treatment should be available at the study center to be administered by trained study center staff if severe hypersensitivity reaction occurs during the ice cube provocation test.

The ice cube provocation test procedure will be described in a separate document.

The primary endpoint will be the proportion of participants with negative ice cube provocation test, defined as the absence of a confluent hive/wheal at the entire skin site of exposure after ice cube provocation test at Week 24. The assessment will be done by the Investigator 10 minutes after ice cube removal. The result will be recorded in eCRF.

### Wheal Intensity Likert Scale

The Wheal intensity Likert scale (ranging from 0 to 5) is a clinician-reported outcome measure comprised of a single item assessing the intensity of patients' cutaneous reaction rated as follows: 0=no wheals; 1=numerous small, non-coalescent wheals; 2=a large, regular, slightly edematous, coalescent wheal; 3=a large and moderately edematous wheal; 4=a large, regular, and significantly edematous wheal without pseudopodia; and 5=a large, very edematous wheal with pseudopodia.

Investigators will complete the scale at the study visit, 10 minutes after removal of the ice cube from the participant's arm. Investigators will complete the wheal intensity Likert scale in eCRF as described in FIG. 2.

### Peak Pruritis Numerical Rating Scale (NRS), Peak Pain NRS, and Peak Burning Sensation NRS

Participants will undergo a temperature provocation test using ice cube at study visits at study center. Ten minutes after the removal of the ice cube from their arm, participants will be asked to rate the severity of local itch, pain, and burning sensation at the provocation site skin using an NRS.

The peak pruritus NRS is a PRO comprised of a single item rated on a scale from 0 (“no itch”) to 10 (“worst itch imaginable”). Participants will be asked to rate the intensity of their worst local site pruritus (itch) 10 minutes after removal of the ice cube. The 24-hour version of the scale has been developed, tested and validated with patients with atopic dermatitis (AD); a threshold value of 4 has been determined as a meaningful within-person change in score in adults and adolescents patients with AD.

The peak pain NRS is a PRO comprised of a single item rated on a scale from 0 (“no pain”) to 10 (“worst imaginable pain”). Participants will be asked to rate the intensity of their worst local site pain 10 minutes after removal of the ice cube.

The peak burning sensation NRS is a PRO comprised of a single item rated on a scale from 0 (“no burning sensation”) to 10 (“worst imaginable burning sensation”). Participants will be asked to rate the intensity of the worst local site burning sensation of their skin 10 minutes after the removal of the ice cube.

Participants will complete the NRS as described in FIG. 2.

### Urticaria Control Test (UCT) 4-Item Version

The UCT is a PRO questionnaire for assessing urticaria control. The questionnaire has been developed and validated with patients with CSU and ColdU. It is comprised of 4 items: severity of physical symptoms of urticaria (itch, hives and/or swelling); QoL impairment; frequency of treatment being not sufficient to control urticaria; overall urticarial control. Recall period is “last four weeks.” Each item is rated on a 5-point Likert scale (scored with 0 to 4 points). Low scores indicate high disease activity and low disease control. The UCT total score is calculated by adding all 4 individual item scores. Accordingly, the minimum and maximum UCT scores are 0 and 16, with a score of 16 for complete disease control.

he MID of the UCT is determined to be 3.

Participants will complete the UCT as described in FIG. 2.

### Cold Urticaria Activity Score (ColdUAS)

The ColdUAS is a disease-specific PRO questionnaire designed to determine cold urticaria disease activity. ColdUAS is intended for patients with cold urticaria aged 12 years old and above; it has been developed and comprehensively tested with adults and adolescent patients with cold urticaria. Disease activity assessment is based on the daily documentation of cold-induced skin reactions (wheals and swelling), skin sensations (itching, burning, pain or feeling hot), avoidance behavior and trigger exposure, and overall symptoms severity. Skin reaction, skin sensations, exposition to cold temperatures that usually cause ColdU symptoms and overall symptom severity are rated on a 4-point scale from 0 (“no”) to 4 (“yes, severe”); avoidance of cold temperatures that usually lead to ColdU symptoms are answered using responses “no,” “yes partially avoided,” “Yes completely avoided.”

After each week of completion, participants will be asked about the overall disease activity of their ColdU in the past week using a 4-point Likert scale from “no disease activity” to “high disease activity.” After each 2 weeks of completion, participants will be asked about the overall disease activity of their ColdU in the past 2 weeks using a 4-point Likert scale from “no disease activity” to “high disease activity,” and they should rate disease activity in the second week compared with first week from “markedly higher than in the first week” to “markedly lower than in the first week.”

Daily ColdUAS are summed over 7-day period to create the ColdUAS7. The ColdUAS7 will be assessed over prior to baseline, week 12, and week 24.

Participants will complete the ColdUAS as described in FIG. 2.

### Dermatology Life Quality Index and Children's Dermatology Life Quality Index (DLOI)

The DLQI is a PRO developed to measure dermatology-specific HRQOL in adult participants. The instrument comprises 10 items assessing the impact of skin disease on participant's HRQOL over the previous week. The items cover symptoms, leisure activities, work/school or holiday time, personal relationships including intimate, the side effects of treatment, and emotional reactions to having a skin disease. It is a validated questionnaire used in clinical practice and clinical trials. The response scale is a 4-point Likert scale (0=“not at all” and 3=“very much”) for 9 items. The remaining 1 item about work/studying asks whether work/study has been prevented and then (if “no”) to what degree the skin condition has been a problem at work/study; the item is rated on a 3-point Likert scale (“not at all” to “a lot”). Overall scoring ranges from 0 to 30, with a high score indicative of a poor HRQOL. Using an integrated analysis of distribution and anchor-based approaches using the change in DLQI total score and participant-assessed itch severity scores, the MID for the DLQI in participants with chronic idiopathic urticaria was reported to be in the range of 2.24 to 3.10 points. So far, there is no MID value determined for ColdU patients.

Participants will complete the DLQI (≥16 years old) as described in FIG. 2.

### Children's DLQI (CDLOI)

The CDLQI is a validated questionnaire designed to measure the impact of skin disease on children's HRQOL. Participants provide responses to 10 questions (symptoms feelings associated with disease, the impact of the disease on leisure, school or holidays, personal relationships, sleep, and side effects of treatment for the skin disease). The instrument has a recall period of 7 days. Nine of the 10 questions are scored on a 4-point Likert scale ranging from 0=not at all/question unanswered to 3=very much. Question 7 has an additional possible response (prevented school), which is assigned a score of 3. The CDLQI total score is the sum of the score of each question with a maximum of 30 and a minimum of 0. The higher the score, the greater the impact is on the child's HRQOL.

Participants will complete the CDLQI (≥12 to <16 years old) as described in FIG. 2.

### Cold Urticaria Quality of Life Questionnaire (ColdU-QoL)

The ColdU-QOL questionnaire is a newly developed disease-specific PRO questionnaire designed to assess the impact of cold urticaria on patients' health-related quality of life. It has been developed and comprehensively tested with adults and adolescent patients with cold urticaria. The questionnaire contains 19 items, each rated using 5-point Likert scale from 0 (not at all/never) to 4 (very much/very often), with a “last 2 weeks” recall period. The total raw score of the ColdU-QOL is transformed to a 0-100 scale with higher scores indicating higher ColdU-related quality of life impairment.

Participants will complete the ColdU-QOL as described in FIG. 2.

### Patient Global Impression of Change (PGIC) of ColdU Disease and Patient Global Impression of Severity (PGIS) of ColdU Disease

The PGIC is a 1-item questionnaire that asks the participant to provide the overall self-assessment of change in their ColdU on a 7-point scale, compared with just before participant started taking the study treatment. Response choices are: 0=“very much better,” 1=“moderately better,” 2=“a little better,” 3=“no change,” 4=“a little worse,” 5=“moderately worse,” 6=“very much worse.”

The PGIS is a 1-item questionnaire that asks participants to provide the overall self-assessment of their ColdU current severity on a 4-point scale. Response choices are: 1=“none,” 2=“mild,” 3=“moderate,” 4=“severe.”

Participants will complete the 2 items after having answered the peak pruritus NRS, pain NRS, and skin burning sensation NRS as described in FIG. 2.

### Acquired Cold Urticaria Severity Index (ACUSI)

The ACUSI is a measure designed to evaluate the severity of the acquired cold urticarial (ACU) signs/symptoms. It is composed of 4 questions regarding the severity of ACU: 1) worst problems ever caused by cold urticarial; 2) season during which problems with outdoor activities occur because it was too cold; 3) maximum treatment needed; 4) frequency of complains. Questions 1, 3, and 4 are attributed 1 to 4 points, and question 3 is attributed 1 to 3 points, thus resulting in a score ranging from 4 to 15. Scores of 4 to 7, 8 to 11, and 12 to 15 points indicate low, middle, and high ACU severity, respectively. A fifth question assesses the overall severity of the disease between mild, moderate, and severe.

The ACUSI questionnaire will be captured in the eCRF.

### EuroQol-5 Dimensions Questionnaire (EQ-5D)

The EQ-5D is a standardized PRO measure of health status developed by the EuroQol Group in order to provide a simple, generic measure of health for clinical and economic appraisal. The EQ-5D consists of 2 parts: the descriptive system (EQ-5D-5L) and the EuroQol visual analogue scale (EQ-VAS). The EQ-5D-5L descriptive system comprises the following 5 dimensions: mobility, self-care, usual activities, pain/discomfort, and anxiety/depression. Each dimension has 5 levels of perceived problems: “no problem,” “slight problems,” “moderate problems,” “severe problems,” and “extreme problems.” The respondent is asked to indicate his/her health state by ticking (or placing a cross) in the box against the most appropriate statement in each of the 5 dimensions; this results in a 1-digit number expressing the level for that dimension. The digits for 5 dimensions can be combined in a 5-digit number describing the respondent's health state. The EQ-VAS records the respondent's self-rated health on a vertical, visual analogue scale (VAS) where the endpoints are labeled “best imaginable health state (100)” and “worst imaginable health state (0).” This information can be used as a quantitative measure of health outcome as judged by the individual respondents.

The recall period is “today.”

Participants will complete the questionnaire as described in FIG. 2.

### Health Care Resource Utilization/Productivity

A questionnaire on health care resource utilization and productivity (missed days of school [12 to 17 years old]/workdays [18 and above]) will be collected by the Investigator for all participants throughout the study through eCRF.

Participants will complete the questionnaire as described in FIG. 2.

### Adverse Events and Serious Adverse Events

### Definition of an AE

An AE is any untoward medical occurrence in a patient or clinical study participant, temporally associated with the use of study intervention, whether or not considered related to the study intervention. Note: An AE can therefore be any unfavorable and unintended sign (including an abnormal laboratory finding), symptom, or disease (new or exacerbated) temporally associated with the use of study intervention.

### Definition of an SAE

A SAE is defined as any untoward medical occurrence that, at any dose: results in death; is life-threatening; requires inpatient hospitalization or prolongation of existing hospitalization; results in persistent disability/incapacity; is a congenital anomaly/birth defect.

An AE will be reported by the participant (or, when appropriate, by a caregiver, parent, surrogate, or the participant's legally authorized representative).

The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

### Time Period and Frequency for Collecting AE and SAE Information

All AEs, serious or nonserious, will be collected from the signing of the ICF until the EOS visit at the time points specified in FIG. 2.

All SAEs and AESIs will be recorded and reported to the Sponsor or designee immediately and under no circumstance should this exceed 24 hours. The Investigator will submit any updated SAE/AESI data to the Sponsor within 24 hours of it being available.

Investigators are not obligated to actively seek AEs or SAEs after conclusion of study participation. However, if the Investigator learns of any SAE, including a death, at any time after a participant has been discharged from the study, and he/she considers the event to be reasonably related to the study intervention or study participation, the Investigator must promptly notify the Sponsor.

### Method of Detecting AEs and SAEs

The method of recording, evaluating, and assessing causality of AEs and SAEs and the procedures for completing and transmitting SAE reports are provided.

Care will be taken not to introduce bias when detecting AEs and/or SAEs. Open-ended and non-leading verbal questioning of the participant is the preferred method to inquire about AE occurrences.

### Follow-Up of AEs and SAEs

After the initial AE/AESI/SAE report, the Investigator is required to proactively follow each participant at subsequent visits/contacts. At the pre-specified study end date, all SAEs, and non-serious AESIs, will be followed until resolution, stabilization, the event is otherwise explained, or the participant is lost to follow-up.

Disease-Related Events and/or Disease-Related Outcomes not Qualifying as AEs or SAEs

An episode of ColdU should be reported as an AE or SAE only if judged by the Investigator to have unexpectedly worsened in severity and/or frequency or change in nature any time during the study. If the participant has a preexisting medical history of angioedema and this condition worsens during the study, it should be reported as an AE or SAE. Any new onset of angioedema in the participant with no prior occurrence should also be reported as an AE or SAE.

Any other AE not listed as an expected event in the IB or in this protocol will be considered unexpected.

### Adverse Event of Special Interest

An AESI is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified, or removed during a study by protocol amendment.

AESIs are as follows: anaphylactic reactions; systemic hypersensitivity reactions; helminthic infections; any severe type of conjunctivitis or blepharitis; keratitis; clinically symptomatic eosinophilia (or eosinophilia associated with clinical symptoms); significant ALT elevation (ALT >5×the ULN in participants with baseline ALT ≤2×ULN; or ALT >8×ULN if baseline ALT >2×ULN); pregnancy of a female participant entered in a study as well as pregnancy occurring in a female partner of a male participant entered in a study with IMP (pregnancy occurring in a female participant entered in the clinical study or in a female partner of a male participant entered in the clinical study. It will be qualified as an SAE only if it fulfills one of the seriousness criteria; in the event of pregnancy in a female participant, IMP should be discontinued; follow-up of the pregnancy in a female participant or in a female partner of a male participant is mandatory until the outcome has been determined); symptomatic overdose (serious or nonserious) with IMP/non-investigational medicinal product (NIMP) (an overdose (accidental or intentional) with the IMP is an event suspected by the Investigator or spontaneously notified by the participant (not based on systematic pills count) and defined as at least twice the intended dose during an interval of less than 11 days. The circumstances (i.e., accidental or intentional) should be clearly specified in the overdose form. An overdose (accidental or intentional) with any NIMP is an event suspected by the Investigator or spontaneously notified by the participant (not based on systematic pills count) and defined as at least twice the maximum prescribed daily dose, within the intended therapeutic interval. The circumstances (i.e., accidental or intentional) should be clearly specified in the overdose form.)

### Anaphylactic Reactions

A serious feature of ColdU is anaphylaxis. Thirty-five percent to 70% of patients with ColdU have experienced systemic reactions including anaphylaxis in severe cases.

Anaphylaxis is defined as a severe, potentially life-threatening systemic hypersensitivity reaction, characterized by being rapid in onset with life-threatening airway, breathing, or circulatory problems that is usually, though not always, associated with skin and mucosal changes.

When the diagnosis of anaphylaxis is made, the basis for having suspected the diagnosis must be documented. Reports of anaphylaxis will be collected as AESIs in the eCRF.

All participants with ColdU and their parents/legal representative/caregiver should be cautioned regarding the risk of anaphylaxis and provided with an epinephrine autoinjector.

The proportion of participants with cold exposure triggered urticaria requiring emergency medical care visit or treatment with epinephrine (after provocation test and/or at home) will be evaluated.

### Pharmacodynamics

Venous blood samples will be collected from all participants for measurement of total serum IgE, which will be done using a validated quantitative method. No other pharmacodynamic parameters will be evaluated in this study.

### Sample Size Determination

To adequately power the study for the comparison of the primary endpoint of the proportion of participants with negative ice cube provocation test at week 24 between the 2 groups, the sample size was calculated based on the following assumptions: the placebo group has 15% of participants with negative ice cube provocation test at week 24 and the dupilumab group has 55% of participants with negative ice cube provocation test at week 24; there is a drop-out rate of 15% in both groups; the statistical test is a Z test that is based on the difference of the 2 proportions with unpooled variance estimate and 2-sided 5% significance level; participants are equally randomized to the dupilumab group and the placebo group.

With these assumptions, 30 participants per group (60 participants in total) will provide 90% power to detect the difference of 55% response rate in the dupilumab group and 15% response rate in the placebo group. The sample size calculations were performed using nQuery+nTerim 4.0.

### Populations for Analyses

The analysis populations are defined in Table 7.

TABLE 7
Analyses Population.

| Population | Description |
|---|---|
| Screened | All participants who sign the ICF |
| Randomized | The randomized population includes all participants with a treatment kit number |
| | allocated and recorded in the IRT database, and regardless of whether the treatment |
| | kit was used or not. |
| | Participants treated without being randomized will not be considered randomized and |
| | will not be included in any efficacy population. |
| Intent-to-treat | All randomized participants analyzed according to the treatment group allocated by |
| (ITT) | randomization. |
| Efficacy | The ITT population |
| Safety | All participants randomly assigned to IMP and who take at least 1 dose of study IMP. |
| | Participants will be analyzed according to the intervention they actually received. |
| | Randomized participants for whom it is unclear whether they took the study IMP will |
| | be included in the safety population as randomized. |
| | For participants who accidentally receive different treatment from the planned, the |
| | actual intervention allocation for as-treated analysis will be the dupilumab group. |
| | The PD analyses will be performed on the safety population. |
| Pharmacokinetic | The PK population indudes all participants in the safety population with at least |
| (PK) | 1 non-missing result for functional dupilumab concentration in serum after first dose of the |
| | IMP. Participants will be analyzed according to the intervention actually received. |
| Antidrug | ADA population includes all participants in the safety population who have at least 1 non- |
| antibody (ADA) | missing ADA result after first dose of the IMP. Participants will be analyzed according to |
| | the intervention actually received. |

Abbreviations:
ADA = antidrug antibody:
ICF = Informed consent form,
IMP = investigational medicinal product:
IRT = Interactive response technology;
ITT = Intent-to-treat;
PD = Pharmacodynamic:
PK = Pharmacokinetic.

### Example 2. A Multi-Center, Single-Arm Study to Investigate the Pharmacokinetics and Safety of Dupilumab in Male and Female Participants ≥2 Years to <12 Years of Age with Uncontrolled Chronic Inducible Cold Urticaria (ColdU)

### Study Rationale

Chronic urticaria is defined by the appearance of itchy wheals (hives) with or without angioedema for more than 6 weeks. Chronic urticaria is divided into chronic inducible urticarias (also called physical urticarias including chronic inducible cold urticaria (ColdU)) and chronic spontaneous urticaria (CSU) for which no triggering factor is identified.

While antihistamines are the mainstay of therapy, up to 50% of patients may remain uncontrolled with antihistamines alone. Treatment of pediatric patients with ColdU remains challenging. The pathophysiology of these conditions is thought to be the same across all age groups, thus antihistamines are first-line therapy. However, there remains a significant unmet need for novel therapies for these indications, particularly in the pediatric population.

### Study Design

The PKM16982 study is a Phase 3, multicenter, single-arm, 24-week treatment study assessing the PK and safety of dupilumab in participants ≥2 years to <12 years of age with ColdU not adequately controlled with H1-antihistamine treatment and/or appropriate preventive measures.

The primary objective of this study is to characterize the PK profile, and the secondary objective is to assess the safety profile of dupilumab in children aged ≥2 years to <12 years with uncontrolled ColdU. This study will additionally collect clinical information regarding the response to treatment in this age group, however all efficacy analyses will be descriptive.

In this study, all enrolled participants will receive dupilumab injections in a weight/age-tiered dosing regimen for 24 weeks, followed by a 12-week post-treatment observational period at the end of the study intervention.

The study consists of three periods:
Screening period (2 to 4 weeks)
Study intervention period (24 weeks)
Follow up period (12 weeks)
The study duration will be 38 to 40 weeks (including screening and follow-up)
The number of study visits will be 8.

### Screening Period

Prior to screening, participants must be receiving treatment for CSU or ColdU with a non-sedating H1-antihistamine. At the screening visit, ColdU participants must have active disease confirmed by a positive ice cube provocation test.

The duration of the screening period will be 2 to 4 weeks.

### Treatment Period

After successful completion of the screening period, participants will begin the treatment period. All participants will be administered dupilumab subcutaneously (SC) every 4 weeks (Q4W) or every 2 weeks (Q2W) with or without an initial loading dose based on weight and age.

### Post-Treatment Period

All participants will complete a 12-week post-treatment follow-up period without study intervention after completing their treatment period.

### Objectives

### Primary Objectives

To characterize the serum concentration of dupilumab over time.

### Secondary Objectives

To assess the safety of dupilumab.
To assess the immunogenicity of dupilumab.
To evaluate the improvement in health-related QoL in participants receiving dupilumab with ColdU who remain symptomatic despite the use of H1-antihistamine.
To assess the impact of dupilumab on urticaria activity in participants with ColdU who remain symptomatic despite the use of H1-antihistamine or appropriate preventive measures.

### Endpoints

Concentration of dupilumab in serum over time including Ctrough at week 12 and week 24.
Safety and tolerability assessments: Incidence of TEAEs or SAEs.
Incidence of ADA to dupilumab over time.
Change from baseline in C-DLQI in children from 4 years to less than 12 years of age at week 24
Change from baseline in IDQOL in children from 2 years to less than 4 years of age at week 24.
Proportion of participants with a negative ice cube provocation test at week 24.
Change from baseline in Wheal Intensity Likert Scale at week 24.

### Inclusion Criteria

Participants are eligible to be included in the study only if all of the following criteria apply:

### Age and Weight

Participant must be ≥2 years to <12 years of age, at the time of signing the informed consent, and have a body weight ≥5 kg to <60 kg.

### Type of Participant and Disease Characteristics

Participants who have a documented diagnosis of ColdU >6 months prior to screening visit.

Participants with ColdU

Participants with ColdU (characterized by recurrent itchy wheals with or without angioedema due to cold for >6 weeks) who remain symptomatic at the time of screening despite the regular or as-needed use of H1-antihistamine or appropriate preventive measures.

Participants with ColdU with a positive ice cube provocation test, presenting with a confluent hive/wheal on the exposed skin area, at the screening visit.

### Compliance

Participant/parent(s)/caregiver(s)/participant's legally authorized representative, as appropriate, willing and able to comply with study visits and related procedures.

### Exclusion Criteria

Participants are excluded from the study if any of the following criteria apply:

### Medical Conditions

Underlying etiology for chronic urticarias other than CSU.

Presence of skin morbidities other than CSU that may interfere with the assessment of the study outcomes.

Participants with a concurrent diagnosis of both CSU and ColdU.

Participants with active AD.

Severe concomitant illness(es) that would adversely affect the patient's participation in the study.

Participants with active tuberculosis (TB) or non-tuberculous mycobacterial infection, or a history of incompletely treated TB.

Diagnosed with, suspected of, or at high risk of endoparasitic infection.

Active chronic or acute infection requiring treatment with systemic antibiotics, antivirals, or antifungals within 2 weeks before the screening visit (V1) or during the screening period.

Known or suspected immunodeficiency.

Active malignancy or history of malignancy within 5 years before the baseline visit.

History of systemic hypersensitivity or anaphylaxis to dupilumab including any excipient.

Participation in prior dupilumab clinical study or have been treated with commercially available dupilumab.

### Physical Examinations

A complete physical examination will include skin, nasal cavities, eyes, ears, respiratory, cardiovascular, gastrointestinal, neurological, lymphatic, and musculoskeletal systems.

Height and weight will also be measured and recorded. Height and weight should be measured with indoor clothing but without shoes.

Investigators should pay special attention to clinical signs related to previous serious illnesses.

Any new finding or worsening of previous finding should be reported as a new AE.

### Vital Signs

Temperature, pulse rate, respiratory rate, and blood pressure will be assessed.

Oral, axillary or tympanic temperature measurement can be used. The same method of temperature measurement should be used during the course of the study.

Blood pressure and pulse measurements will be assessed in a semi-supine or sitting position with a completely automated device. Manual techniques will be used only if an automated device is not available.

Blood pressure and pulse measurements should be preceded by at least 5 minutes of rest for the participant in a quiet setting without distractions (eg, television, cell phones).

### Electrocardiograms

A single standard 12-lead ECG will be obtained as outlined in the SoA. The ECG should be recorded after 10 minutes of rest in the supine position. The ECG will be read locally, and results reported in the eCRF. In case of a clinically significant finding, the Investigator should assess if it impacts a participant's eligibility and document this in the medical records. An AE should be reported if appropriate.

### Clinical Safety Laboratory Tests

The Investigator must review the laboratory report, document this review, and record any clinically significant changes occurring during the study as an AE. The laboratory reports must be filed with the source documents. Abnormal laboratory findings associated with the underlying disease are not considered clinically significant, unless judged by the Investigator to be more severe than expected for the participant's condition.

All laboratory tests with values considered clinically significantly abnormal during participation in the study or within 12 weeks after the last dose of study intervention should be repeated until the values return to normal or baseline or are no longer considered clinically significant by the Investigator.

If clinically significant values do not return to normal/baseline within a period of time judged reasonable by the Investigator, the etiology should be identified and the Sponsor notified.

If laboratory values from non-protocol specified laboratory tests performed at the institution's local laboratory require a change in participant management or are considered clinically significant by the Investigator (eg, SAE or AE or dose modification), then the results must be recorded in the eCRF.

### Pregnancy Testing

Pregnancy testing (urine or serum as required by local regulations) should be conducted in post-menarche female participants at every month.

Pregnancy testing (urine or serum as required by local regulations) should be conducted corresponding with the time frame for female participant contraception.

Post-menarche female (WOCBP) participants will be supplied with urine dipsticks for use between study center visits (starting after visit 5) and will complete the Home Pregnancy Test Diary.

Additional serum or urine pregnancy tests may be performed, as determined necessary by the Investigator or required by local regulation, to establish the absence of pregnancy at any time during the participant's participation in the study.

### Adverse Events (AEs), Serious Adverse Evets (SAEs) and Other Safety Reporting

All AEs (serious or nonserious) will be collected from the signing of the ICF until the follow-up visit.

All SAEs and AESI will be recorded and reported to the Sponsor or designee immediately and under no circumstance should this exceed 24 hours. The Investigator will submit any updated SAE data to the Sponsor within 24 hours of it being available.

Investigators are not obligated to actively seek information on AEs or SAEs after conclusion of the study participation. However, if the Investigator learns of any SAE, including a death, at any time after a participant has been discharged from the study, and he/she considers the event to be reasonably related to the study intervention or study participation, the Investigator must promptly notify the Sponsor.

### Method of Detecting AEs and SAEs

Care will be taken not to introduce bias when detecting AEs and/or SAEs. Open-ended and nonleading verbal questioning of the participant is the preferred method to inquire about AE occurrences.

### Follow-Up of AEs and SAEs

After the initial AE/AESI/SAE report, the Investigator is required to proactively follow each participant at subsequent visits/contacts. At the pre-specified study end-date, all SAEs and AEs of special interest will be followed until resolution, stabilization, the event is otherwise explained, or the participant is lost to follow-up.

### Regulatory Reporting Requirements for SAEs

Prompt notification by the Investigator to the Sponsor of an SAE is essential so that legal obligations and ethical responsibilities towards the safety of participants and the safety of a study intervention under clinical investigation are met.

The Sponsor has a legal responsibility to notify both the local regulatory authority and other regulatory agencies about the safety of a study intervention under clinical investigation. The Sponsor will comply with country-specific regulatory requirements relating to safety reporting to the regulatory authority, IRBs/IECs, and Investigators.

Serious adverse events that are considered expected will be specified in the reference safety information (IB).

Investigator safety reports must be prepared for suspected unexpected serious adverse reactions (SUSARs) according to local regulatory requirements and Sponsor policy and forwarded to the investigators, as necessary.

An Investigator who receives an Investigator safety report describing an SAE, SUSAR or any other specific safety information (e.g., summary or listing of SAEs) from the Sponsor will review and then file it along with the IB and will notify the IRB/IEC, if appropriate according to local requirements. It is the responsibility of the Sponsor to assess whether an event meets the criteria for a SUSAR, and therefore, is expedited to regulatory authorities.

### Pregnancy

Details of all pregnancies in female participants will be collected after the start of study intervention and until 12 weeks (Q2W regimen) or 14 weeks (Q4W regimen) after the last administration of study intervention.

If a pregnancy is reported, the Investigator will record pregnancy information on the appropriate form and submit it to the Sponsor within 24 hours of learning of the pregnancy.

Abnormal pregnancy outcomes (e.g., spontaneous abortion, fetal death, stillbirth, congenital anomalies, ectopic pregnancy) are considered SAEs and will be reported as such.

The participant will be followed to determine the outcome of the pregnancy. The Investigator will collect follow-up information on the participant and the neonate and the information will be forwarded to the Sponsor.

Any post-study pregnancy-related SAE considered reasonably related to the study intervention by the Investigator will be reported to the Sponsor. While the Investigator is not obligated to actively seek this information in former study participants, he or she may learn of an SAE through spontaneous reporting.

Any female participant who becomes pregnant while participating in the study will discontinue study intervention.

Disease-Related Events and/or Disease-Related Outcomes not Qualifying as AEs or SAEs

In participants with ColdU, an episode of ColdU should be reported as an AE or SAE only if judged by the Investigator to have unexpectedly worsened in severity and/or frequency or change in nature any time during the study. If the participant with ColdU has a preexisting medical history of angioedema and this condition worsens during the study, it should be reported as an AE or SAE. Any new onset of angioedema in the participant with no prior occurrence should also be reported as an AE or SAE.

### Adverse Events of Special Interest

An AESI is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified or removed during a study by protocol amendment.

AESIs include: Anaphylactic reactions; Systemic hypersensitivity reactions; Helminthic infections; Any severe type of conjunctivitis or blepharitis; Keratitis; Clinically symptomatic eosinophilia (or eosinophilia associated with clinical symptoms); Increase in ALT (ALT >5×the ULN in participants with baseline ALT≤2×ULN, or ALT >8×ULN if baseline ALT >2×ULN); Pregnancy of a female participant entered in a study with IMP; Symptomatic overdose (serious or nonserious) with IMP/non-investigational medicinal product (NIMP).

### Example 3. Results of Ph3 Study Investigating the Efficacy of Dupilumab in Patients with Chronic Inducible Cold Urticaria (ColdU) Who Remain Symptomatic Despite the Use of H1-Antihistamine Treatment at Week 24-CUrIADS (EFC16720) Study

### Study Overview

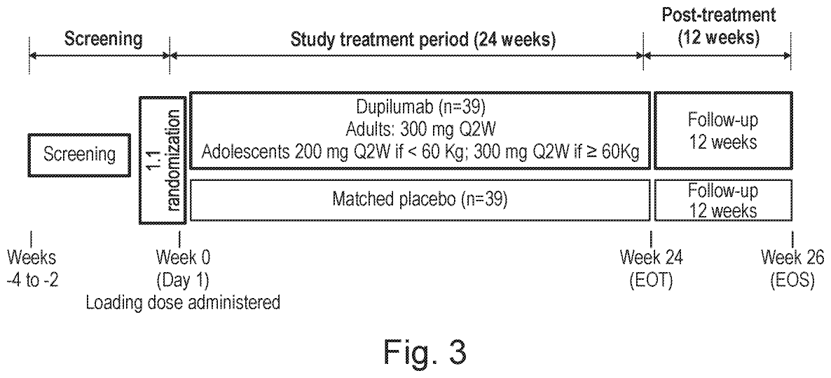

EFC16720 was a phase 3 study evaluating the efficacy and safety of dupilumab in adults and adolescents with chronic inducible cold urticaria who remain symptomatic despite the use of H1-antihistamine treatment. This ColdU study (CUrIADS/EFC16720) consisted of one Ph3 study in adults and adolescents aged ≥12 years (alpha 0.01) (N=82) (FIG. 3). Baseline demographics and disease characteristics are depicted at Tables 8 and 9

TABLE 8
Demographics and participant characteristics
at baseline in a randomized population

| | Placebo | Dupilumab | All |
|---|---|---|---|
| | (N = 40) | (N = 42) | (N = 82) |
| Age (years) | | | |
| Number | 40 | 42 | 82 |
| Mean (SD) | 37.9 (16.3) | 33.0 (13.1) | 35.4 (14.9) |
| Median | 37.5 | 33.5 | 35.0 |
| Q1; Q3 | 25.0; 50.0 | 22.0; 42.0 | 23.0; 45.0 |
| Min; Max | 12; 73 | 13; 58 | 12; 73 |
| Age group [n (%)] | | | |
| Number | 40 | 42 | 82 |
| 12-17 | 4 (10.0) | 4 (9.5) | 8 (9.8) |
| 18-39 | 17 (42.5) | 22 (52.4) | 39 (47.6) |
| 40-64 | 16 (40.0) | 16 (38.1) | 32 (39.0) |
| 65-74 | 3 (7.5) | 0 | 3 (3.7) |
| ≥75 | 0 | 0 | 0 |
| Region⁰ [n (%)] | | | |
| Number | 40 | 42 | 82 |
| Asia | 4 (10.0) | 6 (14.3) | 10 (12.2) |
| Latin America | 9 (22.5) | 11 (26.2) | 20 (24.4) |
| Western Countries | 27 (67.5) | 25 (59.5) | 52 (63.4) |
| Sex [n (%)] | | | |
| Number | 40 | 42 | 82 |
| Male | 10 (25.0) | 9 (21.4) | 19 (23.2) |
| Female | 30 (75.0) | 33 (78.6) | 63 (76.8) |
| Race [n (%)] | | | |
| Number | 40 | 42 | 82 |
| White | 35 (87.5) | 35 (83.3) | 70 (85.4) |
| Black or African | 1 (2.5) | 0 | 1 (1.2) |
| American | | | |
| Asian | 4 (10.0) | 6 (14.3) | 10 (12.2) |
| Japanese | 4 (10.0) | 6 (14.3) | 10 (12.2) |
| Native Hawaiian or | 0 | 0 | 0 |
| Other Pacific Islander | | | |
| American Indian | 0 | 0 | 0 |
| or Alaska Native | | | |
| Multiple | 0 | 1 (2.4) | 1 (1.2) |
| Not reported | 0 | 0 | 0 |
| Unknown | 0 | 0 | 0 |
| Ethnicity [n (%)] | | | |
| Number | 40 | 42 | 82 |
| Hispanic or Latino | 7 (17.5) | 10 (23.8) | 17 (20.7) |
| Not Hispanic or Latino | 31 (77.5) | 31 (73.8) | 62 (75.6) |
| Not reported | 2 (5.0) | 1 (2.4) | 3 (3.7) |
| Weight (kg) | | | |
| Number | 40 | 42 | 82 |
| Mean (SD) | 76.36 (22.01) | 71.28 (16.47) | 73.76 (19.42) |
| Median | 71.65 | 69.50 | 69.75 |
| Q1; Q3 | 62.75; 86.95 | 56.10; 83.40 | 60.00; 86.40 |
| Min; Max | 30.3; 125.7 | 46.0; 106.5 | 30.3; 125.7 |
| Weight group | | | |
| (kg) [n (%)] | | | |
| Number | 40 | 42 | 82 |
| <60 | 8 (20.0) | 12 (28.6) | 20 (24.4) |
| ≥60 | 32 (80.0) | 30 (71.4) | 62 (75.6) |
| BMI (kg/m2) | | | |
| Number | 40 | 42 | 82 |
| Mean (SD) | 28.05 (8.19) | 25.63 (4.99) | 26.81 (6.81) |
| Median | 25.29 | 24.63 | 25.10 |
| Q1; Q3 | 22.99; 31.33 | 21.23; 29.35 | 22.09; 30.30 |
| Min; Max | 14.4; 48.7 | 18.1; 35.3 | 14.4; 48.7 |
| BMI group (kg/m2) | | | |
| [n (%)] | | | |
| Number | 40 | 42 | 82 |
| <30 | 27 (67.5) | 32 (76.2) | 59 (72.0) |
| ≥30 | 13 (32.5) | 10 (23.8) | 23 (28.0) |

BMI: Body mass index
^{a} Asia: Japan; Latin America: Argentina; Western Countries: Canada, USA, Germany.

TABLE 9
Disease and other characteristics at baseline in a randomized population

| | Placebo | Dupilumab | All |
|---|---|---|---|
| | (N = 40) | (N = 42) | (N = 82) |
| Age at onset of cold | | | |
| urticaria (years) | | | |
| Number | 40 | 42 | 82 |
| Mean (SD) | 28.4 | 24.1 | 26.2 |
| | (17.3) | (14.2) | (15.8) |
| Median | 24.5 | 21.0 | 22.5 |
| Q1; Q3 | 14.5; 39.0 | 12.0; 34.0 | 12.0; 35.0 |
| Min; Max | 5; 71 | 4; 58 | 4;71 |
| Age at onset of cold urticaria | | | |
| group (years) [n (%)] | | | |
| Number | 40 | 42 | 82 |
| <18 | 12 (30.0) | 17 (40.5) | 29 (35.4) |
| 18-30 | 13 (32.5) | 11 (26.2) | 24 (29.3) |
| >30 | 15 (37.5) | 14 (33.3) | 29 (35.4) |
| Duration of cold urticaria before | | | |
| screening visit (years)⁰ | | | |
| Number | 40 | 42 | 82 |
| Mean (SD) | 10.0 | 9.4 | 9.7 |
| | (10.1) | (9.0) | (9.5) |
| Median | 7.1 | 7.0 | 7.0 |
| Q1; Q3 | 2.6; 13.0 | 3.0; 14.0 | 3.0; 14.0 |
| Min; Max | 0; 42 | 1; 35 | 0; 42 |
| Duration of cold urticaria before | | | |
| screening visit group (years)⁰ [n (%)] | | | |
| Number | 40 | 42 | 82 |
| <5 | 13 (32.5) | 17 (40.5) | 30 (36.6) |
| 5-10 | 11 (27.5) | 11 (26.2) | 22 (26.8) |
| >10 | 16 (40.0) | 14 (33.3) | 30 (36.6) |
| Number of patients who had | | | |
| cold urticaria events within 12 months | | | |
| before screening visit requiring [n (%)] | | | |
| Number | 9 | 3 | 12 |
| Requiring hospitalization/emergency care | 0 | 1 (33.3) | 1 (8.3) |
| Requiring epinephrine | 0 | 0 | 0 |
| Requiring OCS | 3 (33.3) | 0 | 3 (25.0) |
| Resulting in anaphylactic reaction | 3 (33.3) | 1 (33.3) | 4 (33.3) |
| Resulting in oropharyngeal edema | 3 (33.3) | 1 (33.3) | 4 (33.3) |
| Did the patient experience in past any | | | |
| of the following urticaria symptoms? [n (%)] | | | |
| Number | 40 | 41 | 81 |
| Localized skin urticaria | 36 (90.0) | 37 (90.2) | 73 (90.1) |
| Generalized skin urticaria | 26 (65.0) | 25 (61.0) | 51 (63.0) |
| Localized angioedema, including oropharyngeal | 12 (30.0) | 18 (43.9) | 30 (37.0) |
| Generalized angioedema | 6 (15.0) | 8 (19.5) | 14 (17.3) |
| Other systemic reactions | 11 (27.5) | 14 (34.1) | 25 (30.9) |
| Respiratory distress | 7 (17.5) | 9 (22.0) | 16 (19.8) |
| Hypotension | 5 (12.5) | 9 (22.0) | 14 (17.3) |
| Gastrointestinal discomfort | 2 (5.0) | 4 (9.8) | 6 (7.4) |
| Other | 0 | 0 | 0 |
| History of allergy⁰ | | | |
| Number | 40 | 42 | 82 |
| Allergic | 19 (47.5) | 20 (47.6) | 39 (47.6) |
| Non-Allergic | 21 (52.5) | 22 (52.4) | 43 (52.4) |
| History of angioedema | | | |
| Number | 40 | 42 | 82 |
| Yes | 14 (35.0) | 18 (42.9) | 32 (39.0) |
| No | 26 (65.0) | 24 (57.1) | 50 (61.0) |
| Baseline Wheal intensity Likert scale | | | |
| Number | 40 | 42 | 82 |
| Mean (SD) | 3.3 (1.1) | 3.1 (1.0) | 3.2 (1.0) |
| Median | 3.0 | 3.0 | 3.0 |
| Q1; Q3 | 2.0; 4.0 | 2.0; 4.0 | 2.0; 4.0 |
| Min; Max | 2; 5 | 2; 5 | 2; 5 |
| Baseline Peak pruritus NRS | | | |
| Number | 39 | 40 | 79 |
| Mean (SD) | 6.2 (2.9) | 6.8 (2.7) | 6.5 (2.8) |
| Median | 7.0 | 7.0 | 7.0 |
| Q1; Q3 | 4.0; 8.0 | 6.0; 8.5 | 5.0; 8.0 |
| Min; Max | 0; 10 | 0; 10 | 0; 10 |
| Baseline Peak pain NRS | | | |
| Number | 39 | 40 | 79 |
| Mean (SD) | 5.7 (3.2) | 5.6 (3.2) | 5.6 (3.2) |
| Median | 6.0 | 6.0 | 6.0 |
| Q1; Q3 | 3.0; 8.0 | 3.0; 8.0 | 3.0; 8.0 |
| Min; Max | 0; 10 | 0; 10 | 0; 10 |
| Baseline Peak burning sensation NRS | | | |
| Number | 39 | 40 | 79 |
| Mean (SD) | 5.8 (3.2) | 5.9 (2.9) | 5.8 (3.0) |
| Median | 6.0 | 6.0 | 6.0 |
| Q1; Q3 | 3.0; 8.0 | 5.0; 8.0 | 3.0; 8.0 |
| Min; Max | 0; 10 | 0; 10 | 0; 10 |
| Baseline number of cold exposure | | | |
| days as measured by ColdUAS⁰ | | | |
| Number | 40 | 42 | 82 |
| Mean (SD) | 7.4 (4.0) | 8.6 (4.5) | 8.0 (4.3) |
| Median | 8.0 | 10.0 | 8.0 |
| Q1; Q3 | 5.0; 10.0 | 4.0; 12.0 | 5.0; 12.0 |
| Min; Max | 0; 14 | 0; 14 | 0; 14 |
| Baseline number of days of avoidance | | | |
| to cold as measured by ColdUAS⁰ | | | |
| Number | 40 | 42 | 82 |
| Mean (SD) | 7.3 (5.4) | 8.6 (5.1) | 8.0 (5.2) |
| Median | 8.0 | 10.5 | 9.0 |
| Q1; Q3 | 2.5; 13.0 | 3.0; 13.0 | 3.0; 13.0 |
| Min; Max | 0; 14 | 0; 14 | 0; 14 |
| Baseline number of cold urticaria sign | | | |
| and symptom free days on cold exposure | | | |
| days as measured by ColdUAS⁰ | | | |
| Number | 40 | 42 | 82 |
| Mean (SD) | 0.7 (1.5) | 0.4 (1.5) | 0.5 (1.5) |
| Median | 0.0 | 0.0 | 0.0 |
| Q1; Q3 | 0.0; 0.0 | 0.0; 0.0 | 0.0; 0.0 |
| Min; Max | 0; 5 | 0; 9 | 0; 9 |
| Baseline proportion (%) of cold | | | |
| urticaria sign and symptom free days on cold | | | |
| exposure days as measured by ColdUAS^{0,0} | | | |
| Number | 38 | 41 | 79 |
| Mean (SD) | 7.9 (17.1) | 4.4 (14.7) | 6.1 (15.9) |
| Median | 0.0 | 0.0 | 0.0 |
| Q1; Q3 | 0.0; 0.0 | 0.0; 0.0 | 0.0; 0.0 |
| Min; Max | 0; 63 | 0; 75 | 0; 75 |
| Baseline cold urticaria signs and | | | |
| symptoms severity on cold exposure | | | |
| days as measured by ColdUAS⁰ | | | |
| Number | 38 | 41 | 79 |
| Mean (SD) | 2.6 (1.4) | 2.8 (1.3) | 2.7 (1.3) |
| Median | 2.1 | 2.5 | 2.5 |
| Q1; Q3 | 1.9; 3.0 | 2.0; 3.6 | 1.9; 3.5 |
| Min; Max | 1; 6 | 1; 6 | 1; 6 |
| Baseline UCT | | | |
| Number | 40 | 42 | 82 |
| Mean (SD) | 7.1 (3.9) | 5.4 (3.4) | 6.2 (3.7) |
| Median | 8.0 | 5.5 | 7.0 |
| Q1; Q3 | 5.0; 9.5 | 3.0; 8.0 | 3.0; 9.0 |
| Min; Max | 0; 16 | 0; 13 | 0; 16 |
| Baseline DLQI score | | | |
| Number | 36 | 37 | 73 |
| Mean (SD) | 8.2 (5.1) | 10.8 (8.2) | 9.5 (6.9) |
| Median | 8.0 | 8.0 | 8.0 |
| Q1; Q3 | 4.0; 11.0 | 5.0; 15.0 | 5.0; 13.0 |
| Min; Max | 0; 21 | 1; 30 | 0; 30 |
| Baseline CDLQI score | | | |
| Number | 3 | 3 | 6 |
| Mean (SD) | 14.33 | 9.00 | 11.67 |
| | (12.66) | (6.56) | (9.48) |
| Median | 12.00 | 8.00 | 10.00 |
| Q1; Q3 | 3.00; 28.00 | 3.00; 16.00 | 3.00; 16.00 |
| Min; Max | 3.0; 28.0 | 3.0; 16.0 | 3.0; 28.0 |
| Baseline ColdU-QOL | | | |
| Number | 39 | 40 | 79 |
| Mean (SD) | 41.95 | 47.18 | 44.59 |
| | (24.01) | (24.94) | (24.47) |
| Median | 38.00 | 44.00 | 41.00 |
| Q1; Q3 | 24.00; 63.00 | 28.50; 64.50 | 25.00; 63.00 |
| Min; Max | 4.0; 96.0 | 11.0; 100.0 | 4.0; 100.0 |
| Baseline PGIS score | | | |
| Number | 40 | 42 | 82 |
| Mean (SD) | 2.73 | 3.07 | 2.90 |
| | (0.82) | (0.71) | (0.78) |
| Median | 3.00 | 3.00 | 3.00 |
| Q1; Q3 | 2.00; 3.00 | 3.00; 4.00 | 2.00; 3.00 |
| Min; Max | 1.0; 4.0 | 2.0; 4.0 | 1.0; 4.0 |
| Screening ACUSI | | | |
| Number | 40 | 39 | 79 |
| Mean (SD) | 9.45 | 9.79 | 9.62 |
| | (2.19) | (1.76) | (1.99) |
| Median | 9.00 | 10.00 | 9.00 |
| Q1; Q3 | 8.00; 11.00 | 9.00; 11.00 | 8.00; 11.00 |
| Min; Max | 4.0; 15.0 | 6.0; 15.0 | 4.0; 15.0 |
| Screening ACUSI categorical | | | |
| score [n (%)] | | | |
| Number | 40 | 39 | 79 |
| 4-7 (Mild ACU) | 7 (17.5) | 3 (7.7) | 10 (12.7) |
| 8-11 (Moderate ACU) | 26 (65.0) | 30 (76.9) | 56 (70.9) |
| 12-15 (Severe ACU) | 7 (17.5) | 6 (15.4) | 13 (16.5) |
| Worst problems ever caused | | | |
| by cold urticaria [n (%)] | | | |
| Number | 40 | 39 | 79 |
| Hive, redness or itching | 25 (62.5) | 18 (46.2) | 43 (54.4) |
| Deep swelling of the skin or mucous | 4 (10.0) | 9 (23.1) | 13 (16.5) |
| membranes/Angioedema | | | |
| Circulatory complaints/ | 9 (22.5) | 10 (25.6) | 19 (24.1) |
| Dizziness/Difficulty | | | |
| swallowing/Difficulty breathing | | | |
| Unconsciousness/Shock | 2 (5.0) | 2 (5.1) | 4 (5.1) |
| During which season the | | | |
| problem with outdoor activities | | | |
| occurs because of cold [n (%)] | | | |
| Number | 40 | 39 | 79 |
| Only in winter | 5 (12.5) | 5 (12.8) | 10 (12.7) |
| Winter, spring and autumn | 7 (17.5) | 11 (28.2) | 18 (22.8) |
| During all seasons | 28 (70.0) | 23 (59.0) | 51 (64.6) |
| Maximum treatment needed for | | | |
| the cold urticaria [n (%)] | | | |
| Number | 40 | 39 | 79 |
| Avoid cold | 3 (7.5) | 1 (2.6) | 4 (5.1) |
| Antihistamines | 30 (75.0) | 32 (82.1) | 62 (78.5) |
| Emergency medication use | 4 (10.0) | 2 (5.1) | 6 (7.6) |
| (Cortisone/Adrenaline) | | | |
| Emergency physician | 3 (7.5) | 4 (10.3) | 7 (8.9) |
| Frequency of complaining about | | | |
| the cold urticaria when at | | | |
| its worst [n (%)] | | | |
| Number | 40 | 39 | 79 |
| Rarely | 4 (10.0) | 3 (7.7) | 7 (8.9) |
| Occasionally | 8 (20.0) | 5 (12.8) | 13 (16.5) |
| Often | 12 (30.0) | 12 (30.8) | 24 (30.4) |
| Daily or almost daily | 16 (40.0) | 19 (48.7) | 35 (44.3) |
| Patients' assessment of the severity | | | |
| of cold urticaria [n (%)] | | | |
| Number | 40 | 39 | 79 |
| Mild | 5 (12.5) | 6 (15.4) | 11 (13.9) |
| Moderate | 26 (65.0) | 19 (48.7) | 45 (57.0) |
| Severe | 9 (22.5) | 14 (35.9) | 23 (29.1) |
| Baseline EQ-5D-5L single index score⁰ | | | |
| Number | 39 | 40 | 79 |
| Mean (SD) | 0.80 | 0.83 | 0.82 |
| | (0.19) | (0.21) | (0.20) |
| Median | 0.84 | 0.88 | 0.84 |
| Q1; Q3 | 0.70; 1.00 | 0.72; 1.00 | 0.71; 1.00 |
| Min; Max | 0.0; 1.0 | 0.0; 1.0 | 0.0; 1.0 |
| Baseline EQ-VAS | | | |
| Number | 39 | 40 | 79 |
| Mean (SD) | 69.77 | 73.88 | 71.85 |
| | (25.68) | (23.69) | (24.62) |
| Median | 80.00 | 82.00 | 80.00 |
| Q1; Q3 | 50.00; 90.00 | 65.00; 90.00 | 60.00; 90.00 |
| Min; Max | 2.0; 100.0 | 8.0; 100.0 | 2.0; 100.0 |
| Baseline Total IgE (IU/mL) | | | |
| Number | 38 | 41 | 79 |
| Mean (SD) | 415.43 | 487.06 | 452.60 |
| | (728.45) | (741.79) | (731.57) |
| Median | 154.00 | 173.00 | 165.00 |
| Q1; Q3 | 33.30; 618.00 | 63.30; 489.00 | 48.60; 596.00 |
| Min; Max | 3.4; 4123.0 | 1.0; 2926.0 | 1.0; 4123.0 |
| Baseline Total IgE (IU/mL) [n (%)] | | | |
| Number | 38 | 41 | 79 |
| <100 | 18 (47.4) | 15 (36.6) | 33 (41.8) |
| ≥100 | 20 (52.6) | 26 (63.4) | 46 (58.2) |
| Baseline H1-antihistamine use [n (%)] | | | |
| Number | 40 | 42 | 82 |
| Regular/daily use | 24 (60.0) | 29 (69.0) | 53 (64.6) |
| Per-need use | 16 (40.0) | 13 (31.0) | 29 (35.4) |
| Baseline H1-antihistamine dose | | | |
| if regular/daily use [n (%)] | | | |
| Number | 24 | 29 | 53 |
| <1-fold | 1 (4.2) | 0 | 1 (1.9) |
| 1-fold | 13 (54.2) | 20 (69.0) | 33 (62.3) |
| 2-fold to 3-fold | 10 (41.7) | 6 (20.7) | 16 (30.2) |
| 4-fold | 0 | 3 (10.3) | 3 (5.7) |
| Prior cold urticaria medication use [n (%)] | | | |
| Number | 5 | 6 | 11 |
| Leukotriene receptor antagonists (LTRAs) | 2 (40.0) | 4 (66.7) | 6 (54.5) |
| H2-blockers | 2 (40.0) | 2 (33.3) | 4 (36.4) |
| Omalizumab | 1 (20.0) | 1 (16.7) | 2 (18.2) |

OCS: oral corticosteroids;
UCT: urticaria control test;
NRS: numerical rating scale;
ColdUAS: cold urticaria activity score;
DLQI: Dermatology Life Quality Index;
CDLQI: Children's Dermatology Life Quality Index;
ColdU-QOL: Cold Urticaria Quality of Life;
PGIS: participant global impression of severity;
ACUSI: Acquired Cold Urticaria Severity Index;
EQ-5D-5L: 5-level EuroQol 5-dimensional questionnaire;
EQ-VAS: EuroQol visual analogue scale.
^{a}Derived as (Year of screening visit-Year of first diagnosis of cold urticaria) + (month of screening visit-month of first diagnosis of cold urticaria)/12
^{b}Defined as having a medical history of chronic spontaneous urticaria, angioedema (not related to cold urticaria), atopic dermatitis, allergic rhinitis, allergic conjunctivitis, asthma, food allergy, chronic sinusitis, nasal polyps or eosinophilic esophagitis.
cThe ColdUAS related assessments were conducted during each 14 days window. The e-diary completion days should be >6 and cold exposure days should be >1 in the 14 days window, otherwise the assessment result is set to missing.
^{d}Proportion of cold urticaria sign and symptom free days = sign and symptom free days/cold exposure days in 14 days window *100
^{e}EQ-5D-5L health status was converted into a single index value by using EQ-5D-5L value sets based on UK population.
Note:
A low score indicates good outcome for wheal intensity Likert scale (range 0-5), peak pruritus NRS (range 0-10), peak pain NRS (range 0-10), Peak burning sensation NRS (range 0-10), cold urticaria signs and symptoms severity on cold exposure days (range 0-6), DLQI/CDLQI (range 0-30), ColdU-QoL (range 0-100), PGIS (range 1-4), and ACUSI (rang 4-15); A high score indicates good outcome for UCT (range 0-16) and EQ-VAS (range 0-100).

### Key Inclusion Criteria

Age: ≥12 years to 80 years of age.

ColdU diagnosis/confirmation of uncontrolled disease.

Diagnosis of ColdU >6 weeks prior to screening visit.

Positive ice cube provocation test at screening and randomization visit.

Presence of one of the following: UCT<12 at screening and randomization visit; or within 6 months prior to screening visit, cold exposure triggered anaphylaxis, oropharyngeal edema, cold exposure triggered urticaria requiring emergency medical care visit or treatment with epinephrine.

### Key Exclusion Criteria

Subjects with atopic dermatitis.

Subjects with chronic spontaneous urticaria.

Subjects with non-ColdU inducible urticarias.

### Endpoints

Ice cube provocation: standardized/validated, local reading.

Wheal Intensity Likert scale: COA, validation ongoing, local reading.

ColdUAS: PRO, validation ongoing, e-diary.

### Summary of Results at Week 24

Dupilumab treatment did not show statistically significant improvement versus placebo for the primary endpoint and across key secondary and other multiplicity-adjusted secondary endpoints that evaluated key components of ColdU, including hives and itch and quality of life. A numerical difference was observed in favor of the dupilumab group as compared to the placebo group in the ColdUAS proportion of ColdU signs and symptoms free days and ColdU signs and symptoms severity scores on cold exposure days. However, given the amount of missing data (data imputed in 40% of participants in the dupilumab group and 30% of participants in the placebo group), no conclusion could be drawn.

Overall, there were no notable imbalances in: drop-out rate between dupilumab and placebo; baseline demographics and disease characteristics; H1-AH background/rescue use.

### Limitations of the Present Study

A small sample size (N=82) and increased drop-out rate (26%).

A lack of validated ColdU specific measures.

The prior ColdU studies were conducted at academic centers, with limited number of sites and patients (rupatadine, Xolair).

### Ice Cube Provocation Test

The ice cube provocation test should be performed by applying a cold stimulus (e.g., ice cube, cool packs) to forearm skin. Cold provocation testing should be performed for 1 to 5 minutes on volar forearm. Ice cube should be melting within a thin plastic bag to avoid cold damage of the skin and to prevent direct water contact to avoid the confusion with aquagenic urticaria if the test is positive. Reading time: 10 minutes of rewarming after ice cube is removed. A negative test is defined as the absence of a confluent hive/wheal at the entire skin site of exposure after ice cube provocation test.

Negative ice cube provocation test at week 24:17 [40.5%] in the dupilumab group versus 15 [37.5%] in the placebo group; OR: 1.03, p=0.9492).

### Wheal Intensity Likert Scale

The wheal intensity Likert scale is a clinician-reported outcome measure comprised of a single item assessing the intensity of a patient's cutaneous reaction ten minutes after removal of the ice cube. No midpoint (MID) has been established for ColdU for the wheal intensity Likert scale.

For the local wheal intensity Likert scale (a key secondary endpoint), the LS mean difference versus placebo was −0.03, with 95% CI: −0.72, 0.66 (p=0.9375).

### Cold Urticaria Activity Score (ColdUAS)

ColdUAS is a disease specific PRO-questionnaire designed to determine cold urticaria disease activity in subjects with ColdU. Disease activity assessment is based on the daily documentation of cold-induced skin reactions (wheals and swelling), skin sensations (itching, burning, pain, or feeling hot), avoidance behavior and trigger exposure, and overall symptoms severity. Skin reactions, skin sensations, exposure to cold temperatures that usually cause ColdU symptoms and overall symptom severity are rated on a 4-point scale from 0 (“No”) to 4 (“Yes, severe”); avoidance of cold temperatures that usually lead to ColdU symptoms are answered using responses: “no,” “yes partially avoided,” or “yes completely avoided.”

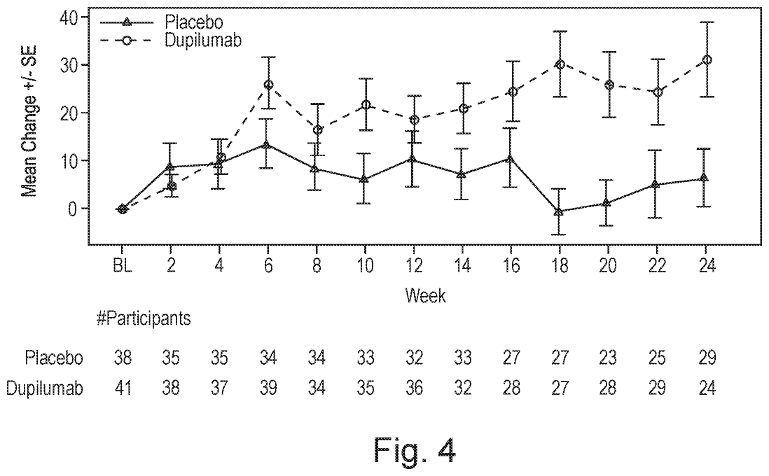

For proportion of cold urticaria sign- and symptom-free days on cold exposure days (a key secondary endpoint), the LS mean difference versus placebo was +12.16, with 95% CI: −5.81, 30.12 (p=0.1846) (FIG. 4).

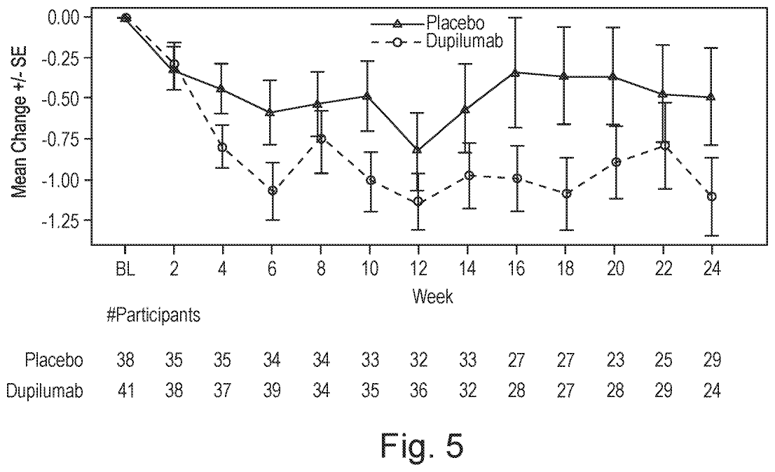

Another secondary endpoint was cold urticaria sign and symptom severity score on cold exposure days at week 24: the LS mean difference versus placebo was −0.24, with 95% CI: −0.78, 0.30) (p=0.3823) (FIG. 5).

### Peak Pruritus Numerical Rating Scale

The peak pruritis numerical rating scale is a patient-reported outcome (PRO) comprised of a single item rated on a scale from 0 (“no itch”) to 10 (“worst itch imaginable”). Participants were asked to rate the intensity of their worst local site pruritis (itch) ten minutes after removal of the ice cube. No midpoint (MID) has been established for ColdU for the peak pruritis numerical rating scale.

For local itch severity NRS, the LS mean difference versus placebo was-0.24, with 95% CI: −1.82, 1.33 (p=0.7598).

### Dermatology Life Quality Index

The Dermatology Life Quality Index (DLQI) is a PRO developed to measure dermatology-specific HRQOL in adult participants. It comprises 10 items assessing the impact of skin disease on participant's HRQOL over the previous week. The items cover symptoms, leisure activities, work/school or holiday time, personal relationships including intimate, the side effects of treatment, and emotional reactions to having a skin disease. The response scale is a 4-point Likert scale (0=“not at all” and 3=“very much”) for 9 items. The remaining 1 item about work/studying asks whether work/study has been prevented and then (if “no”) to what degree the skin condition has been a problem at work/study; the item is rated on a 3-point Likert scale (“not at all” to “a lot”). Overall scoring ranges from 0 to 30, with a high score indicative of a poor HRQOL.

For DLQI, the LS mean difference versus placebo was +0.39, with 95% CI: −2.33, 3.11 (p=0.7798). (FIG. 5).

### Total IgE Change Over Time

Total IgE change over time shows the levels of serum IgE concentration in a subject over time that can include a baseline value and values for one or more time points during treatment.

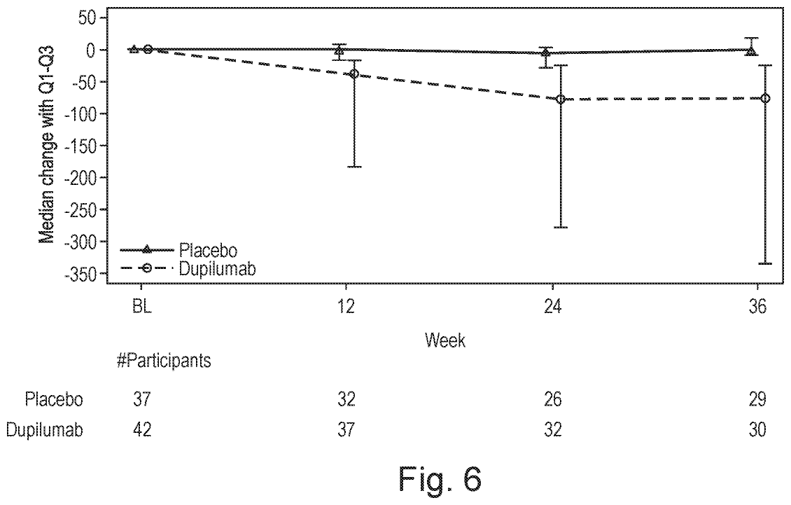

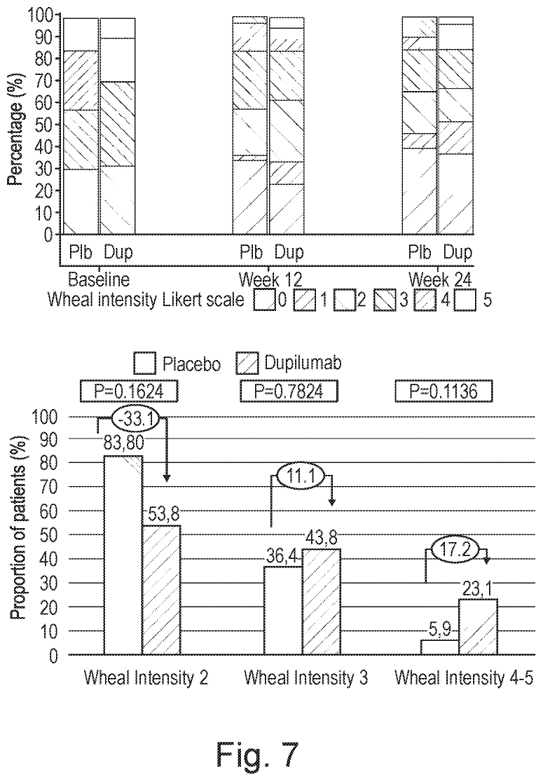

In a safety population, a 56% reduction from baseline was observed for dupilumab vs. placebo (FIG. 6).

### Safety Summary

Dupilumab was well-tolerated and demonstrated an acceptable safety profile in ColdU patients (N=40 placebo; 42 dupilumab). The safety profile was consistent with the known safety profile of dupilumab observed in the approved populations and indications. The overall incidence of TEAEs was balanced between placebo vs. dupilumab. The incidence of TEAEs leading to treatment discontinuation, SAE and AESI was low: there was a higher incidence of injection site reactions with dupilumab (3 placebo vs. 11 dupilumab); 1 AESI (keratitis) on placebo, conjunctivitis was balanced between placebo and dupilumab (1 placebo vs. 1 dupilumab); COVID-19 related events were balanced between placebo and dupilumab (11 placebo vs. 9 dupilumab); and the safety in eight adolescents was comparable to adults.

What is claimed is:

1. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and
wherein cold urticaria activity (ColdUAS) score is improved in the subject.

2. The method of claim 1, wherein the subject remains symptomatic despite the use of H1 antihistamine.

3. The method of claim 1 or 2, wherein an H1 antihistamine is administered in combination with the antibody or an antigen-binding fragment thereof.

4. The method of claim 3, wherein the H1 antihistamine is selected from the group consisting of cetirizine, levocetirizine, ebastine, fexofenadine, loratadine, desloratadine, bilastine, and rupatadine.

5. The method of any one of claims 1-4, wherein the subject is an adult.

6. The method of claim 5, wherein the antibody or antigen-binding fragment thereof is administered to the subject as an initial dose followed by one or more secondary doses.

7. The method of claim 6, wherein the initial dose is about 600 mg and each secondary dose is about 300 mg.

8. The method of claim 7, wherein each secondary dose is administered every 2 weeks.

9. The method of any one of claims 1-4, wherein the subject is 12 years old to less than 18 years old.

10. The method of claim 9, wherein the antibody or antigen-binding fragment thereof is administered to the subject as an initial dose followed by one or more secondary doses.

11. The method of claim 10, wherein the subject has a body weight greater than or equal to 30 kg and less than 60 kg and wherein the initial dose is about 400 mg and each secondary dose is about 200 mg.

12. The method of claim 11, wherein each secondary dose is administered every 2 weeks.

13. The method of claim 12, wherein the subject has a body weight of at least 60 kg and wherein the initial dose is about 600 mg and each secondary dose is about 300 mg.

14. The method of claim 13, wherein each secondary dose is administered every 2 weeks.

15. The method of any one of claims 1-4, wherein the subject is 6 years old to less than 12 years old.

16. The method of any one of claims 1-4, wherein the subject is 2 years old to less than 12 years old.

17. The method of claim 15 or 16, wherein the antibody or antigen-binding fragment thereof is administered to the subject as an initial dose followed by one or more secondary doses.

18. The method of claim 17, wherein the subject has a body weight of at least 30 kg and wherein the initial dose is about 400 mg and each secondary dose is about 200 mg.

19. The method of claim 18, wherein each secondary dose is administered every 2 weeks.

20. The method of claim 19, wherein the subject has a body weight of less than 30 kg and at least 15 kg and wherein the initial dose is about 600 mg and each secondary dose is about 300 mg.

21. The method of claim 20, wherein each secondary dose is administered every 4 weeks.

22. The method of claim 21, wherein the subject has a body weight of less than 30 kg and at least 15 kg and wherein the initial dose is about 300 mg and each secondary dose is about 300 mg.

23. The method of claim 22, wherein each secondary dose is administered every 4 weeks.

24. The method of claim 23, wherein the subject is at least 2 years old and less than 6 years old.

25. The method of claim 17, wherein the subject has a body weight of less than 15 kg and at least 5 kg and wherein the initial dose is about 200 mg and each secondary dose is about 200 mg.

26. The method of claim 25, wherein each secondary dose is administered every 4 weeks.

27. The method of claim 26, wherein the subject is at least 2 years old and less than 6 years old.

28. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is 12 years old to less than 18 years old,
wherein the antibody or antigen-binding fragment thereof is administered to the subject as an initial dose followed by one or more secondary doses,
wherein the antibody or antigen-binding fragment thereof is administered to the subject in a weight-dependent dosage, and
wherein cold urticaria activity (ColdUAS) score is improved in the subject.

29. The method of claim 28, wherein the subject has a body weight greater than or equal to 30 kg and less than 60 kg and wherein the initial dose is about 400 mg and each secondary dose is about 200 mg.

30. The method of claim 28, wherein the subject has a body weight of at least 60 kg and wherein the initial dose is about 600 mg and each secondary dose is about 300 mg.

31. The method of claim 29 or 30, wherein each secondary dose is administered every 2 weeks.

32. The method of any one of claims 28-31, wherein an H1 antihistamine is administered in combination with the antibody or an antigen-binding fragment thereof.

33. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is the subject is 12 years old to less than 18 years old,
wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with antihistamine therapy, and
wherein cold urticaria activity (ColdUAS) score is improved in the subject.

34. The method of claim 33, wherein the subject has a body weight greater than or equal to 30 kg and less than 60 kg and wherein the initial dose is about 400 mg and each secondary dose is about 200 mg.

35. The method of claim 33, wherein the subject has a body weight of at least 60 kg and wherein the initial dose is about 600 mg and each secondary dose is about 300 mg.

36. The method of claim 34 or 35, wherein each secondary dose is administered every 2 weeks.

37. The method of any one of claims 33-36, wherein the subject remains symptomatic despite the use of H1 antihistamine.

38. The method of any one of claims 33-37, wherein an H1 antihistamine is administered in combination with the antibody or an antigen-binding fragment thereof.

39. The method of claim 37 or 38, wherein the H1 antihistamine is selected from the group consisting of cetirizine, levocetirizine, fexofenadine, loratadine, desloratadine, bilastine, and rupatadine.

40. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is the subject is 6 years old to less than 12 years old,
wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with H1 antihistamine therapy, and
wherein cold urticaria activity (ColdUAS) score is improved in the subject.

41. The method of claim 40, wherein the subject has a body weight of between 15 kg and less than 30 kg, and wherein the initial dose is about 600 mg and each secondary dose is about 300 mg.

42. The method of claim 41, wherein each secondary dose is administered every 4 weeks.

43. The method of any one of claims 40-42, wherein the subject remains symptomatic despite the use of H1 antihistamine.

44. The method of any one of claims 40-43, wherein an H1 antihistamine is administered in combination with the antibody or an antigen-binding fragment thereof.

45. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject has a body weight of between 30 kg and less than 60 kg, wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with H1 antihistamine therapy, and
wherein cold urticaria activity (ColdUAS) score is improved in the subject.

46. The method of claim 45, the initial dose is about 400 mg and each secondary dose is about 200 mg.

47. The method of claim 45, wherein each secondary dose is administered every 2 weeks.

48. The method of claim 45, wherein the subject is at least 6 years old but less than 12 years old.

49. The method of any one of claims 45-48, wherein the subject remains symptomatic despite the use of H1 antihistamine.

50. The method of any one of claims 45-49, wherein an H1 antihistamine is administered in combination with the antibody or an antigen-binding fragment thereof.

51. The method of claim 49 or 50, wherein the H1 antihistamine is selected from the group consisting of cetirizine, levocetirizine, fexofenadine, loratadine, desloratadine, bilastine, and rupatadine.

52. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject has a body weight of between 5 kg and less than 15 kg,
wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with H1 antihistamine therapy, and
wherein cold urticaria activity (ColdUAS) score is improved in the subject.

53. The method of claim 52, the initial dose is about 200 mg and each secondary dose is about 200 mg.

54. The method of claim 52, wherein each secondary dose is administered every 4 weeks.

55. The method of claim 52, wherein the subject is at least 2 years old but less than 6 years old.

56. The method of any one of claims 52-55, wherein the subject remains symptomatic despite the use of H1 antihistamine.

57. The method of any one of claims 52-56, wherein an H1 antihistamine is administered in combination with the antibody or an antigen-binding fragment thereof.

58. The method of claim 56 or 57, wherein the H1 antihistamine is selected from the group consisting of cetirizine, levocetirizine, fexofenadine, loratadine, desloratadine, bilastine, and rupatadine.

59. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject has a body weight of between 15 kg and less than 30 kg,
wherein the subject is at least 2 years old but less than 6 years old,
wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with H1 antihistamine therapy, and
wherein cold urticaria activity (ColdUAS) score is improved in the subject.

60. The method of claim 59, the initial dose is about 300 mg and each secondary dose is about 300 mg.

61. The method of claim 59, wherein each secondary dose is administered every 4 weeks.

62. The method of any one of claims 59-61, wherein the subject remains symptomatic despite the use of H1 antihistamine.

63. The method of any one of claims 59-62, wherein an H1 antihistamine is administered in combination with the antibody or an antigen-binding fragment thereof.

64. The method of claim 62 or 63 wherein the H1 antihistamine is selected from the group consisting of cetirizine, levocetirizine, fexofenadine, loratadine, desloratadine, bilastine, and rupatadine.

65. A method for treating a subject having chronic inducible cold urticaria (ColdU) comprising administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the subject is the subject is 2 years old to less than 12 years old,
wherein the antibody or antigen-binding fragment thereof is administered to the subject as an initial dose followed by one or more secondary doses,
wherein the antibody or antigen-binding fragment thereof is administered to the subject in a weight dependent dosage,
wherein the subject was previously ineffectively treated with H1 antihistamine therapy, and
wherein cold urticaria activity (ColdUAS) score is improved in the subject.

66. The method of claim 65, wherein the subject has a body weight of between 5 kg and less than 15 kg, and wherein the initial dose is about 200 mg and each secondary dose is about 200 mg.

67. The method of claim 66, wherein each secondary dose is administered every 4 weeks.

68. The method of claim 65, wherein the subject is at least 2 years old but less than 6 years old.

69. The method of claim 65 or 68, wherein the subject has a body weight of between 15 kg and less than 30 kg, and wherein the initial dose is about 300 mg and each secondary dose is about 300 mg.

70. The method of claim 69, wherein each secondary dose is administered every 4 weeks.

71. The method of claim 65, wherein the subject is at least 6 years old but less than 12 years old.

72. The method of claim 65 or 71, wherein the subject has a body weight of between 15 kg and less than 30 kg, and wherein the initial dose is about 600 mg and each secondary dose is about 300 mg.

73. The method of claim 72, wherein each secondary dose is administered every 4 weeks.

74. The method of claim 65, wherein the subject has a body weight of between 30 kg and less than 60 kg, and wherein the initial dose is about 400 mg and each secondary dose is about 200 mg.

75. The method of claim 74, wherein each secondary dose is administered every 2 weeks.

76. The method of any one of claims 65-75, wherein the subject remains symptomatic despite the use of H1 antihistamine.

77. The method of any one of claims 65-76, wherein an H1 antihistamine is administered in combination with the antibody or an antigen-binding fragment thereof.

78. The method of any of the preceding claims, wherein the treatment results in an improvement in one or more ColdU-related outcomes selected from the group consisting of ice cube provocation test score, wheal intensity Likert scale score, peak pruritus numerical rating scale (NRS) score, peak pain NRS score, peak burning sensation NRS score, urticaria control test (UCT) 4-item version score, children's dermatology quality life quality index (CDLQI) score, infants' dermatitis quality of life index (IDQOL) score, patient global impression of change (PGIC) score, patient global impression of severity (PGIS) score, acquired cold urticaria severity index (AColdUSI) score, 5-level EuroQol 5-dimensional questionnaire (EQ-5D-5L) score, health care resource utilization/productivity score.

79. The method of claim 78, wherein the improvement in the one or more ColdU-associated measures occurs with 12 weeks of treatment with the antibody or antigen-binding fragment thereof.

80. The method of any one of claims 1-78, wherein the improvement in the one or more ColdU-associated measures occurs with 24 weeks of treatment with the antibody or antigen-binding fragment thereof.

81. The method of any of the preceding claims, wherein prior to treatment with the antibody or antigen-binding fragment thereof, the subject has angioedema.

82. The method of any of the preceding claims, wherein the treatment with the antibody or antigen-binding fragment thereof results in an increase in the number of itch-free days that the subject experiences.

83. The method of any of the preceding claims, wherein the treatment with the antibody or antigen-binding fragment thereof results in an increase in the number of hive-free days that the subject experiences.

84. The method of any of the preceding claims, wherein the treatment with the antibody or antigen-binding fragment thereof results in a decrease in the need for treatment of the subject with oral corticosteroids.

85. The method of claim 84, wherein the dosage of oral corticosteroids required is decreased.

86. The method of claim 84 or 85, wherein the number of days wherein oral corticosteroid treatment is required is decreased.

87. The method of any of the preceding claims, wherein the treatment with the antibody or antigen-binding fragment thereof results in a decrease in the need for treatment of the subject with antihistamine rescue medication.

88. The method of claim 87, wherein the dosage of antihistamine rescue medication required is decreased.

89. The method of claim 87 or 88, wherein the number of days wherein antihistamine rescue medication is required is decreased.

90. The method of any of the preceding claims, wherein the antibody or antigen-binding fragment thereof comprises a heavy chain variable region (HCVR) sequence of SEQ ID NO:
1 and a light chain variable region (LCVR) sequence of SEQ ID NO: 2.

91. The method of any one of the preceding claims, wherein the antibody is dupilumab.

92. The method of any of the preceding claims, wherein the antibody or antigen-binding fragment thereof is administered using an autoinjector, a needle and syringe, or a pen.

93. The method of any one of the preceding claims, wherein the antibody or antigen-binding fragment thereof is administered using a prefilled device.

94. The method of any one of the preceding claims, wherein the antibody or antigen-binding fragment thereof is administered subcutaneously.

95. A method for treating chronic inducible cold urticaria (ColdU) in a subject comprising:
selecting a subject having ColdU; and
administering to the subject an antibody or an antigen-binding fragment thereof that specifically binds interleukin-4 receptor (IL-4R),
wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and
wherein cold urticaria activity (ColdUAS) score is improved in the subject.

96. The method of claim 95, wherein prior to treatment with the antibody or antigen-binding fragment thereof, the subject has angioedema.

97. The method of claim 95, wherein the treatment with the antibody or antigen-binding fragment thereof results in an increase in the number of itch-free days that the subject experiences.

98. The method of claim 95, wherein the treatment with the antibody or antigen-binding fragment thereof results in an increase in the number of hive-free days that the subject experiences.

99. The method of claim 95, wherein the treatment with the antibody or antigen-binding fragment thereof results in a decrease in the need for treatment of the subject with oral corticosteroids.

100. The method of any of the preceding claims, wherein the ColdUAS score is measured by sign and symptom free days on cold exposure days or is measured by sign and symptom severity score on cold exposure days.

101. The method of claim 100, wherein the ColdUAS score is decreased with 24 weeks of treatment with the antibody or antigen-binding fragment thereof.

102. The method of any of the preceding claims, wherein total serum IgE is reduced in the subject.

103. The method of claim 102, wherein the total serum IgE is reduced with 24 weeks of treatment with the antibody or antigen-binding fragment thereof.
